(19) United States
(12) Patent Application Publication
(10) Pub. No.: US 2021/0139892 A1
(43) Pub. Date: May 13, 2021

(54) COMPOSITIONS AND METHODS FOR DELIVERY OF RNA TO A CELL

(71) Applicant: The Regents of the University of California, Oakland, CA (US)

(72) Inventors: Ross Crawford Wilson, Berkeley, CA (US); Colleen Amanda McGourty, Berkeley, CA (US); Lorena de Oñate Monje, Berkeley, CA (US)

(21) Appl. No.: 17/253,536

(22) PCT Filed: Jul. 2, 2019
(86) PCT No.: PCT/US19/40301

§ 371 Date: Dec. 17, 2020

Related U.S. Application Data
(60) Provisional application No. 62/694,212, filed on Jul. 5, 2018.

(51) Int. Cl. C12N 15/11; C12N 9/22

(52) U.S. Cl. CPC C12N 15/11; C12N 9/22; C12N 2320/30; C12N 2310/3513; C12N 2310/20

(57) ABSTRACT

The present disclosure provides a system comprising: a) a modified RNA-binding protein (RBP) comprising: i) an RBP; and ii) one or more endosomolytic peptides (ELPs) covalently linked, directly or via a linker, to the RBP; and b) a modified cargo RNA complexed to the RBP, wherein the modified cargo RNA comprises a cargo RNA modified to include one or more RBP binding sites that are bound by the RBP present in the modified RBP. The present disclosure also provides a method of delivering a cargo RNA to a eukaryotic cell, the method comprising contacting the cell with the system of the present disclosure. The present disclosure provides methods of delivering a cargo RNA to the cytoplasm of a cell. The present disclosure provides methods of delivering a cargo RNA to the cytosol of a cell.

### CROSS-REFERENCE

This application claims the benefit of U.S. Provisional Patent Application No. 62/694,212, filed Jul. 5, 2018, which application is incorporated herein by reference in its entirety.

### INCORPORATION BY REFERENCE OF SEQUENCE LISTING PROVIDED AS A TEXT FILE

A Sequence Listing is provided herewith as a text file, “BERK-386WO_SEQ_LISTING ST25.txt” created on Jul. 1, 2019 and having a size of 7,676 KB. The contents of the text file are incorporated by reference herein in their entirety.

### INTRODUCTION

RNA-guided endonuclease (RGEN) technology has enabled precise alteration of the genome in living cells, a process known as genome editing. RGEN-enabled genome editing has rapidly transformed biomedical research and holds immense promise for the treatment, prevention, or curing of genetic disease as well as infectious disease. However, translating genome editing into effective medical therapies is limited by the ability to deliver RNA-guided CRISPR/Cas effector proteins, such as RGEN enzymes, to tissue in an effective, safe, and non-toxic manner.

One approach for tissue-specific delivery of RNA-guided CRISPR/Cas effector proteins is to co-localize them with an agent that promotes endocytosis. Such a strategy has been used to carry out tissue-specific uptake of cytotoxic anti-cancer drugs coupled to receptor-specific antibodies (Lambert et al., Adv. Ther. 2017; 34(5): 1015-1035), as well as tissue-targeted delivery of therapeutic siRNA (Willoughby et al., Mol Ther. 2018; 26(1); 105-114). However, tissue-specific endocytosis represents only the first step for receptor-facilitated macromolecular delivery. The second step is to promote escape of the macromolecular cargo from the endosome. Without endosomal escape, the macromolecular cargo is trafficked to the lysosome, where it is degraded before it has the opportunity to take therapeutic action within the cell. Various reagents, such as chloroquine, polyethyleneimine, certain highly charged cationic compounds, cell penetrating peptides (CPPs), and inactivated adenoviruses, have been developed that are intended to quickly disrupt the endosome in order to minimize the amount of time that a delivered bioactive agent spends in the endosome-like environment. However, these agents often lack generality and present suboptimal ability to promote endosomal escape of the cargo and are associated with various disadvantages, including toxicity problems. The challenge of efficient and non-toxic endosomal escape has been a substantial barrier in the delivery of macromolecular therapeutics (Varkouhi et al., Journal of Controlled Release 2011; 151(3); 220-228), especially for in vivo applications.

Another approach to the RNA-guided CRISPR/Cas effector protein delivery and internalization involve adeno-associated virus, liposomes, or lipid nanoparticles. These methods are limited in their efficacy because they are based on non-tissue-specific association with the cell surface and as such will be internalized by many cell types.

Thus, there is a need in the art for compositions and methods for delivering RNA-guided CRISPR/Cas effector proteins to cells, where such effector proteins may be complexed with guide RNA.

### SUMMARY

The present disclosure provides a system comprising: a) a modified RNA-binding protein (RBP) comprising: i) an RBP; and ii) one or more endosomolytic peptides (ELPs) covalently linked, directly or via a linker, to the RBP; and b) a modified cargo RNA complexed to the RBP, wherein the modified cargo RNA comprises a cargo RNA modified to include one or more RBP binding sites that are bound by the RBP present in the modified RBP. The present disclosure also provides a method of delivering a cargo RNA to a eukaryotic cell, the method comprising contacting the cell with the system of the present disclosure. The present disclosure provides methods of delivering a cargo RNA to the cytoplasm of a cell. The present disclosure provides methods of delivering a cargo RNA to the cytosol of a cell.

### BRIEF DESCRIPTION OF THE DRAWINGS

FIG. 1 provides the amino acid sequences for U1A construct mutations.

FIG. 2 illustrates features of a system of the present disclosure, including: the protein component of a ribonucleoprotein (RNP); a modified cargo RNA that includes binding sites which are recognized by a corresponding RBP component; and RBPs that have been chemically conjugated to an endosomolytic agent (ELA), e.g., an endosomolytic peptide (ELP).

FIG. 3 illustrates an example of the subject system including: a) a CRISPR/Cas effector polypeptide (e.g., Cas9); a Cas9 single-guide RNA (sgRNA) modified to contain three RNA-based binding sites for the RBP domain U1A; and U1A polypeptides that have been covalently conjugated to an ELA (e.g., an ELP).

FIG. 4 illustrates EMX1 editing results in HEPG2 cells, which were treated with a positive control for each RNP construct (“noSL” conditions), and RNPs containing various sgRNA designs capable of recruiting various numbers of U1A RBP adaptor molecules to their engineered U1A binding sites, wherein the U1A RBP adaptor molecules include 1, 2, or 3 covalently bound ELPs (labelled “ELA” in the figure).

FIG. 5A-5F provides amino acid sequences of Streptococcus pyogenes Cas9 (FIG. 5A) and variants of Streptococcus pyogenes Cas9 (FIG. 5B-5F).

FIG. 6 provides an amino acid sequence of Staphylococcus aureus Cas9.

FIG. 7A-7C provide amino acid sequences of Francisella tularensis Cpf1 (FIG. 7A), Acidaminococcus sp. BV3L6 Cpf1 (FIG. 7B), and a variant Cpf1 (FIG. 7C).

FIG. 8 depicts a sodium dodecyl sulfate-polyacrylamide gel electrophoresis (SDS-PAGE) gel, showing the mobility of U1A and its derivatives that have been conjugated with the pyridyl disulfide-functionalized variant of the ppTG21 peptide.

FIG. 9 depicts a trace from mass spectrometry analysis of U1A variants, where the variants are conjugated with one, two, or three chains of ppTG21.

FIGS. 10A-10B depict the strategy and results for guide RNA production.

FIGS. 11A-11B depict mass spectrometry traces of guide RNAs.

FIGS. 12A-12B are graphs from fluorescence polarization (FP) binding assays

FIGS. 13A-13B are graphs from FP binding assays testing for affinity between adaptor protein U1A and different guide RNA constructs.

FIG. 14 is a trace from a biolayer interferometry (BLI) experiment, showing the absence of substantial binding between U1A and a Cas9 RNP containing an unmodified guide RNA (noSL).

FIGS. 15A-15D depict a trace from a biolayer interferometry (BLI) results demonstrating persisting binding between U1A and a Cas9 RNP containing an engineered guide RNA bearing three binding sites for the U1A adaptor (noSL).

FIGS. 16A-16E depict a graph demonstrating receptor-mediated genome editing of the EMX1 gene via adaptor-recruited endosomolytic peptides (arELP) bound to Cas9 RNP.

### DEFINITIONS

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “antibodies” and “immunoglobulin” include antibodies or immunoglobulins of any isotype, fragments of antibodies that retain specific binding to antigen, including, but not limited to, Fab, Fv, single-chain Fv (scFv), and Fd fragments, chimeric antibodies, humanized antibodies, single-chain antibodies (scAb), single domain antibodies (dAb), single domain heavy chain antibodies, single domain light chain antibodies, nanobodies, bi-specific antibodies, and multi-specific antibodies.

The term “analog” of an amino acid residue refers to a residue having a sidechain group that is a structural and/or functional analog of the sidechain group of the reference amino acid residue. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as F, Cl or Br, etc.), deletion of a group, substitution of a covalent bond (single bond for double bond, etc.), or combinations thereof. For example, amino acid analogs may include α-hydroxy acids, and α-amino acids, and the like. In some cases, an analog of an amino acid residue is a substituted version of the amino acid. The term “substituted version” of an amino acid residue refers to a residue having a sidechain group that includes one or more additional substituents on the sidechain group that are not present in the sidechain of the reference amino acid residue.

The terms “heterocyclic amino acid” and “heterocyclic residue” are used interchangeably to refer to an amino acid residue where the sidechain group includes a heterocyclic group, such as a heteroaryl group or a saturated heterocyclic group. In some cases, the sidechain group is a heterocycle-alkyl or a substituted heterocycle-alkyl group. The terms are meant to include naturally occurring and non-naturally occurring alpha-amino acids. Naturally occurring heterocyclic residues of interest include tryptophan and histidine. In some cases, the amino acid group is “pH responsive”. The term “pH responsive,” as used herein, refers to an amino acid whose side chain is responsive to the local pH such that the side chain is differentially protonated under select conditions. pH responsiveness is dependent on the pKa of the amino acid. Non-limiting examples include histidine (pKa˜6.0) and glutamic acid (pKa˜4.5). pH responsive amino acids are often found in endosomolytic peptides.

The terms “non-polar amino acid residue” and “non-polar residue” refer to an amino acid residue that includes a sidechain that is hydrogen (i.e., G) or a non-polar group. In some cases, a non-polar amino acid sidechain is a hydrophobic group. The terms are meant to include naturally occurring and non-naturally occurring alpha-amino acids. Naturally occurring non-polar amino acid residues of interest include naturally occurring hydrophobic residues.

The terms “hydrophobic amino acid” and “hydrophobic residue” are used interchangeably to refer to an amino acid residue where the sidechain group is a hydrophobic group. The terms are meant to include naturally occurring and non-naturally occurring alpha-amino acids. Naturally occurring hydrophobic residues of interest include alanine, isoleucine, leucine, phenylalanine, proline and valine.

The terms “polar amino acid” and “polar residue” are used interchangeably to refer to an amino acid residue where the sidechain group includes a polar group or charged group. In certain cases, the polar group is capable of being a hydrogen bond donor or acceptor. The terms are meant to include naturally occurring and non-naturally occurring alpha-amino acids. Naturally occurring polar residues of interest include arginine, asparagine, aspartic acid, histidine, lysine, serine, threonine, tyrosine, cysteine, methionine, glutamic acid, glutamine and tryptophan.

In some cases, a polar amino acid group is a cationic amino acid group. The term “cationic amino acid” refers to a positively charged amino acid. Particular examples include but are not limited to, lysine, arginine and histidine.

The terms “scaffold” and “scaffold domain” are used interchangeably and refer to a reference RBP motif from which a subject RBP arose, or against which the subject RBP is able to be compared, e.g., via a sequence or structural alignment method. The structural motif of a scaffold domain can be based on a naturally occurring protein domain structure. For a particular protein domain structural motif, several related underlying sequences may be available, any one of which can provide for the particular three-dimensional structure of the scaffold domain.

The terms “parent amino acid sequence,” “parent sequence,” and “parent polypeptide” refer to a polypeptide comprising an amino acid sequence from which a variant peptidic compound arose and against which the variant peptidic compound is being compared. The parent polypeptide lacks one or more of the modifications or variant amino acids disclosed herein and can differ in function compared to a variant peptidic compound as disclosed herein.

The terms “corresponding residue” and “residue corresponding to” are used to refer to an amino acid residue located at equivalent positions of variant and parent sequences.

As used herein, the terms “variant amino acid” and “variant residue” are used interchangeably to refer to the particular residues of a subject compound which are modified or mutated by comparison to an underlying scaffold domain. The variant residues encompass those residues that were selected (e.g., via mirror image screening, affinity maturation and/or point mutation(s)) to provide for a desirable domain motif structure that specifically binds to the target. When a compound includes amino acid mutations or modifications at particular positions by comparison to a scaffold domain, the amino acid residues of the peptidic compound located at those particular positions are referred to as “variant amino acids.” Such variant amino acids may confer on the resulting peptidic compounds different functions, such as specific binding to a target protein, increased water solubility, ease of chemical synthesis, metabolic stability, etc.

The terms “variant domain” and “variant motif” refers to an arrangement of variant amino acids incorporated at particular locations of a scaffold domain. The variant motif can encompass a continuous and/or a discontinuous sequence of residues. The variant motif can encompass variant amino acids located at one face of the compound structure. The variant domain may be considered to be incorporated into, or integrated with, an underlying scaffold domain structure or sequence. In the subject compounds, the scaffold domain can provide a stable three-dimensional protein structural motif, e.g., of a naturally occurring protein domain, while the variant domain can be defined by an arrangement of characteristic minimum number of variant residues at a modified surface of the structure that is capable of specifically binding a target protein.

The term “mutation” refers to a deletion, insertion, or substitution of an amino acid(s) residue or nucleotide(s) residue relative to a reference sequence, such as a scaffold sequence.

A “modified” protein refers to herein as a protein that has been altered to differ from the protein in its original form. Non-limiting examples of modifications of proteins may include incorporation of amino acid analogs, deletion and/or insertion of amino acids in the primary sequence of the protein, incorporation of chemical moieties (e.g. addition of small chemical groups such as methyl or hydroxyls and/or linkage to large moieties such as PEGylation chains or other large chemical structures), and conjugation (e.g., directly or via a linker) to a second molecule, e.g., fusion with another protein as compared to the protein in its original form.

The term “domain” refers to a continuous or discontinuous sequence of amino acid residues. A domain can include one or more regions or segments. The terms “region” and “segment” are used interchangeably to refer to a continuous sequence of amino acid residues that, in some cases, can define a particular secondary structural feature.

The term “naturally-occurring” as used herein as applied to a nucleic acid, a protein, a cell, or an organism, refers to a nucleic acid, cell, protein, or organism that is found in nature.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

“Heterologous,” as used herein, refers to a nucleotide or amino acid sequence that is not found in the native nucleic acid or protein, respectively. For example, relative to a Cas9 polypeptide, a heterologous polypeptide comprises an amino acid sequence from a protein other than the Cas9 polypeptide.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, nucleotide sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant nucleotide sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such artificial combination can be carried out to join together nucleic acid segments of desired functions to generate a desired combination of functions.

Similarly, the term “recombinant” polypeptide refers to a polypeptide which is not naturally occurring, e.g., is made by the artificial combination of two otherwise separated segments of amino acid sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change can be achieved by introduction of new DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector), and include the progeny of the original cell which has been genetically modified by the nucleic acid. In some cases, a host cell is present in vivo within an organism. Non-limiting examples of a host cell in vivo within an organism include a liver cell, a blood cell, a muscle cell, or the like within an organ or tissue of the organism. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Any convenient linking groups can be utilized in the subject systems. The terms “linker”, “linkage” and “linking group” are used interchangeably and refer to a linking moiety that covalently connects two or more compounds. In some cases, the linker is divalent. In certain cases, the linker is a branched or trivalent linking group. In some cases, the linker has a linear or branched backbone of 200 atoms or less (such as 100 atoms or less, 80 atoms or less, 60 atoms or less, 50 atoms or less, 40 atoms or less, 30 atoms or less, or even 20 atoms or less) in length. A linking moiety may be a covalent bond that connects two groups or a linear or branched chain of between 1 and 200 atoms in length, for example of about 1, 2, 3, 4, 5, 6, 8, 10, 12, 14, 16, 18, 20, 30, 40, 50, 100, 150 or 200 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, oligo(ethylene glycol), ethers, thioethers, disulfide, amides, carbonates, carbamates, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. A linker may be peptidic, e.g., a linking sequence of residues.

As used herein, the term “cleavable linker” refers to a linker that can be selectively cleaved to produce two products. Application of suitable cleavage conditions to a molecule containing a cleavable linker that is cleaved by the cleavage conditions will produce two byproducts. A cleavable linker of the present disclosure is stable, e.g. to physiological conditions, until it is contacted with a cleavage-inducing stimulus, e.g., an agent such as an enzyme or other cleavage-inducing agent or stimulus such as chemical agent, irradiation with light and/or heat. In some cases, the cleavable linker is photocleavable, i.e., cleavable upon irradiation with light of a suitable wavelength. In some cases, the cleavable linker is thermally cleavable, i.e., cleavable upon heating to a suitable temperature. Exemplary conditions are set forth below.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a guide RNA” includes a plurality of such guide RNAs and reference to “the RNA-guided CRISPR/Cas effector protein” includes reference to one or more RNA-guided CRISPR/Cas effector proteins and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides systems and methods for delivery of an RNA cargo to the cytoplasm of a eukaryotic cell. The present disclosure provides systems and methods for delivery of an RNA cargo to the cytosol of a eukaryotic cell. The present disclosure provides systems and methods for delivery of an RNA cargo from the cytosol to the nucleus of a eukaryotic cell. The present disclosure provides a system comprising: a) a modified RNA-binding protein (RBP) comprising: i) an RBP; and ii) one or more endosomolytic peptides covalently linked, directly or via a linker, to the RBP; and b) a modified cargo RNA complexed to the RBP, wherein the modified cargo RNA comprises a cargo RNA that is modified to include one or more RBP binding sites that are bound by the RBP present in the modified RBP. The present disclosure provides a system comprising: a) a modified RBP comprising: i) an RBP; and ii) one or more cell penetrating peptides and/or endosomolytic peptides covalently linked, directly or via a linker, to the RBP; and b) a modified cargo RNA complexed to the RBP, wherein the modified cargo RNA comprises a cargo RNA that is modified to include one or more RBP binding sites that are bound by the RBP present in the modified RBP. The present disclosure also provides a method of delivering a cargo RNA to a eukaryotic cell, the method comprising contacting the cell with the system of the present disclosure.

### System

The present disclosure provides systems for delivery of a cargo RNA to the cytoplasm (e.g., to the cytosol and/or to an intracellular organelle such as the nucleus) of a eukaryotic cell. The subject systems are useful for facilitating controlled tissue-specific delivery of a cargo RNA. A system of the present disclosure can induce endocytosis of the system by a eukaryotic cell and subsequent endosomal escape to the cytoplasm (e.g., to the cytosol). In some cases, a system of the present disclosure includes: i) a modified cargo RNA comprising a cargo RNA that is modified to include one or more binding sites for an RNA-binding protein (RBP); and ii) a modified RBP complexed to the binding sites in the cargo RNA, wherein the RBP has been modified by covalently linking one or more endosomolytic peptides (ELPs) to one or more locations in the RBP. In some cases, a system of the present disclosure includes: i) a modified cargo RNA comprising a cargo RNA that is modified to include one or more binding sites for an RBP; and ii) a modified RBP complexed to the binding sites in the cargo RNA, wherein the RBP has been modified by covalently linking one or more cell penetrating peptides (CPPs) and/or ELPs to one or more locations in the RBP. In some cases, the ELP is covalently bonded directly to the RBP of interest. In other cases, the ELP is covalently bonded to the RBP via a linker, such as an acid-labile or a reduction-labile linker to facilitate timely release of the ELP once the cargo is inside the endosome. In some cases, the modified cargo RNA comprises a guide RNA that is modified to include one or more binding sites for the RBP portion of the modified RBP. In some cases, the system includes an RNA-guided CRISPR/Cas effector protein, where the RNA-guided CRISPR/Cas effector protein is complexed with the modified guide RNA. It is understood that any convenient RNA sequence that has been engineered to include sequences that are capable of recruiting RBPs that have been covalently modified with ELPs can be utilized in the subject systems.

As disclosed herein the ELPs include polypeptide sequences capable of promoting transit of co-localized macromolecular cargo from the endosome (or other cellular compartment) to the cytosol. In some cases, ELPs comprise about 5 to about 60 amino acid residues. In certain cases, ELPs comprise about 20 to about 25 amino acid residues. In some cases, the ELPs are rich in histidine residues. Any convenient ELP capable of promoting transit of co-localized macromolecular cargo from the endosome to the cytosol can be used in the present systems. It will be understood that ELPs are distinct from “cell penetrating peptides” (CPPs), which promote internalization from the cell exterior to the cytosol. By contrast to CPPs, ELPs promote endosomal escape, a process following endocytosis that prevents cargo from being trafficked to the lysosome for rapid degradation.

As disclosed herein, the ELP is covalently bound either directly or indirectly via a linker to the RBP of interest. It will be understood that conjugation of the ELP to the RBP may be achieved by any convenient methodology. In certain cases, the conjugation of the ELP to the RBP is performed using disulfide chemistry at cysteine residues that are not naturally occurring but have been incorporated in the RBP and ELP at sites of intended modification. The subject RBP is chosen to give this system the versatility to co-localize a precise number of ELPs to the cargo RNA complex. Through the variable number of RBP binding sites in the cargo RNA as well as the variable number of ELPs conjugated to the RBP itself, the system permits control of the number of endosomal escape-promoting polypeptides being co-localized with the cargo RNA. Such versatility will allow systems to be identified and developed so as to achieve the correct balance between effective endosomal escape (more polypeptides) and safety/specificity (fewer peptides). The number and/or identity of ELPs utilized in the system will depend on the cargo RNA being delivered and/or the tissue to which the cargo RNA is being delivered. The flexibility and modularity of the subject system allows it to be quickly adapted for multiple biomedical applications.

The subject systems provide a means of imparting a cargo RNA with the ability to escape the endosomes of mammalian cells by co-localizing it with a precisely controlled number of ELPs. This will facilitate development of cargo RNA-based therapeutics, which currently lack methods of delivery that are effective and tissue specific.

Accordingly, in some cases, the present disclosure provides a system comprising: a) a modified RNA-binding protein (RBP) comprising: i) an RBP; and ii) one or more endosomolytic peptides covalently linked, directly or via a linker, to the RBP; and b) a modified cargo RNA complexed to the RBP, wherein the modified cargo RNA comprises a cargo RNA that is modified to include one or more RBP binding sites that are bound by the RBP present in the modified RBP.

In some cases, the system is described by the formula:

A(X-(L-Z)ₙ)ₘ

wherein, A is a cargo RNA modified to include one or more binding sites for complexing to one or more modified RBPs represented by (X-(L-Z)ₙ)ₘ; X is an RBP of interest (e.g., as described herein); L is an optional linking group (e.g., as described herein); Z is an ELP of interest (e.g., as described herein); and n and m are each independently an integer from 1 to 10. In some cases, the cargo RNA (A) comprises one or more binding sites for complexing a modified RBP, such that m is 1, 2, 3, 4, 5, 6, 7, 8, 9 or 10 binding sites. In some cases, the cargo RNA (A) comprises 10 or less binding sites for complexing a modified RBP, such that m is 10, 9, 8, 7, 6, 5, 4, 3, 2 or 1. In certain cases, the cargo RNA (A) comprises 5 binding sites for complexing a modified RBP. In other cases, the cargo RNA (A) comprises 4 binding sites for complexing a modified RBP. In certain cases, the cargo RNA (A) comprises 3 binding sites for complexing a modified RBP. In some cases, the cargo RNA (A) comprises 2 binding sites for complexing a modified RBP. In other cases, the cargo RNA (A) comprises 1 binding site for complexing a modified RBP. In some embodiments of the cargo RNA (A), further comprises a binding site for a polypeptide other than the modified RBP.

In some embodiments of the modified RBPs described by (X-(L-Z)ₙ)ₘ, one or more ELPs of interest are covalently attached to X, each independently and optionally via a linking group L, such that n is 1, 2, 3, 4, 5, 6, 7, 8, 9 or 10. In some cases, 10 or fewer ELPs of interest are covalently attached to X, each independently and optionally via a linking group L, such that n is 10, 9, 8, 7, 6, 5, 4, 3, 2 or 1. In certain instances 5 ELPs of interest are covalently attached to X, each independently and optionally via a linking group L. In certain instances, 4 ELPs of interest are covalently attached to X, each independently and optionally via a linking group L. In certain instances, 3 ELPs of interest are covalently attached to X, each independently and optionally via a linking group L. In certain instances, 2 ELPs of interest are covalently attached to X, each independently and optionally via a linking group L. In certain instances, 1 ELP of interest is covalently attached to X, optionally via a linking group L. In some instances, the linking group L is a branched linker. In some instances, one or more ELPs (e.g., 1 or more, 2 or more, 3 or more, 4 or more, 5 or more, 6 or more, 7 or more, 8 or more, 9 or more, or 10 or more) of interest are covalently attached to X via one or more branched linkers.

In some embodiments, the cargo RNA (A) comprises 1-3 binding sites complexed to 1-3 modified RBPs of interest (i.e., m is 1, 2 or 3) described by (X-(L-Z)ₙ)ₘ, wherein each X is covalently attached to 1-3 ELPs of interest (i.e., n is 1, 2 or 3) optionally and independently via a linking group L.

In some cases, the one or more endosomolytic peptides is activated at low pH. The term “activated,” as used herein, in its conventional sense, refers to a peptide that has been modified (e.g. transiently or permanently) in a manner such that it is able to perform a function. For example, an activated endosomolytic peptide is able to promote endosomal escape of the modified RBP.

In some cases, the one or more endosomolytic peptides comprises an amino acid sequence selected from GLFHALLHLLHSLWHLLLHA (SEQ ID NO: 817), GLFEAIAEFIENGWEGLIEGWYGGRKKRRQRRR (SEQ ID NO: 818), GPSQPTYPGDDAPVRDLIRFYRDLRRY (SEQ ID NO: 819), WYSCNVCGKAFVLSRHLNRHLRVHRRAT (SEQ ID NO: 820) and HHEHHEHHEHHEHHEHHEHHEHHEHHE (SEQ ID NO: 821).

In some embodiments of the system, the RBP of the modified RBP is linked to the endosomolytic peptide via a cleavable linker. In certain cases, the cleavable linker is an enzyme-cleavable linker, an acid-cleavable linker, a reduction-cleavable linker or a redox-cleavable linker.

In some cases, the modified RBP comprises 1 endosomolytic peptide. In some cases, the modified RBP comprises 2 endosomolytic peptides. In other cases, the modified RBP comprises 3 endosomolytic peptides. In yet other cases, the modified RBP comprises 4 endosomolytic peptides. In some cases, the modified RBP comprises one or more endosomolytic peptides selected from any one of the amino acid sequences set forth in SEQ ID NOs: 818, 819, 820, and 821. In some cases, the one or more endosomolytic peptides comprises an amino acid sequence set forth in SEQ ID NO:818. In some cases, the one or more endosomolytic peptides comprises an amino acid sequence set forth in SEQ ID NO:819. In some cases, the one or more endosomolytic peptides comprises an amino acid sequence set forth in SEQ ID NO:820. In some cases, the one or more endosomolytic peptides comprises an amino acid sequence set forth in SEQ ID NO:21. In some cases, the one or more endosomolytic peptides comprises any one of the amino acid sequences set forth in SEQ ID NO:818-821, or a combination thereof.

In some cases, the cargo RNA comprises a binding site for a polypeptide other than the modified RBP.

In some cases, the cargo RNA is a guide RNA comprising: i) a targeting segment comprising a nucleotide sequence that hybridizes to a nucleotide sequence in a target nucleic acid; and ii) an activation segment comprising a nucleotide sequence that binds to and activates an RNA-guided effector enzyme.

In some cases, the system comprises an RNA-guided CRISRP/Cas effector polypeptide. In certain cases, the RNA-guided CRISRP/Cas effector polypeptide is a class 2 CRISPR/Cas effector polypeptide. In some cases, the class 2 CRISPR/Cas effector polypeptide is a type II CRISPR/Cas effector polypeptide. In certain cases, the class 2 CRISPR/Cas effector polypeptide is a Cas9 protein and the corresponding CRISPR/Cas guide RNA is a Cas9 guide RNA. In some cases, the class 2 CRISPR/Cas effector polypeptide is a type V or type VI CRISPR/Cas effector polypeptide. In certain cases, the class 2 CRISPR/Cas effector polypeptide is a Cpf1 protein, a C2c1 protein, a C2c3 protein, or a C2c2 protein. In some cases, the class 2 CRISPR/Cas effector polypeptide is a Cas12 enzyme (e.g., Cas12a (Cpf1), Cas12e (CasX), Cas12b1 (C2c1), Cas12c (C2c3), or Cas12d (CasY)). In other cases, the class 2 CRISPR/Cas effector polypeptide is a Cas13 enzyme (e.g., Cas13a (C2c2), Cas13d, Cas13c (C2c7), or Cas13b (C2c6)). Non-limiting examples of Cas13 enzymes can be found in Makarova et al. (CRISPR J (2018) 1(5):325-336), which is hereby incorporated by reference in its entirety. In some cases, the RNA-guided CRISRP/Cas effector polypeptide is a fusion polypeptide comprising: a) the RNA-guided CRISRP/Cas effector polypeptide; and b) a fusion partner, where the fusion partner is a base editor. In some cases, the RNA-guided CRISRP/Cas effector polypeptide is a fusion polypeptide comprising: a) the RNA-guided CRISRP/Cas effector polypeptide; and b) a fusion partner, where the fusion partner is a FokI nuclease.

In some embodiments of the system, the guide RNA comprises one or more nucleic acid modifications. In certain cases, the one or more nucleic acid modifications comprise one or more of a modified nucleobase, a modified backbone or non-natural internucleoside linkage, a modified sugar moiety, a Locked Nucleic Acid, and a Peptide Nucleic acid.

In some embodiments of the system, the RNA-guided CRISRP/Cas effector polypeptide is a fusion polypeptide comprising: i) the RNA-guided effector polypeptide; and b) a fusion partner, where the fusion partner is a targeting moiety covalently linked to the RNA-guided CRISRP/Cas effector polypeptide. In certain cases, the targeting moiety is an antibody, a ligand-binding portion of a receptor, or a ligand for a receptor.

The methods and compositions described herein can be used to deliver any RNA cargo to a cell. In some instances, the ELPs described in the present disclosure can be substituted with CPPs. For example, CPPs can be used to deliver cargo RNAs directly into the cytosol. Thus, CPPs conjugated to an RNA binding protein as described herein may be used to deliver cargo RNAs. The majority of CPPs mainly include arginine and lysine residues, making them cationic and hydrophilic. In some instances, CPPs can be amphiphilic, anionic, or hydrophobic in nature. CPPs are conventionally composed of 6-30 amino acids and, due to the short sequence length, they can be easily synthesized according to methods well known in the art. Studies have revealed that the positive charge and amphipathic nature of CPPs are the critical features for cellular internalization and allow CPPs to carry macromolecules, polypeptides, and oligonucleotides across the cell membrane. Some CPPs are derived from natural biomolecules (e.g. Tat, an HIV-1 protein), while others (e.g., polyarginine) are obtained by synthetic methods. Non-limiting examples of CPPs include HIV-1 TAT (GRKKRRQRRRPPQ; SEQ ID NO:885); Penetratin pAntp (RQIKIWFQNRRMKWKK; SEQ ID NO:886); polyarginines; HRSV (RRIPNRRPRR; SEQ ID NO:887); AIP6 (RLRWR; SEQ ID NO:888); MPG (GALFLGFLGAAGSTMGAWSQPKKKRKV; SEQ ID NO:889); Pep-1 (KETWWETWWTEWSQPKKRKV; SEQ ID NO:890); ARF(1-22) MVRRFLVTLRIRRACGPPRVRV; SEQ ID NO:891); pVEC (LLIILRRRIRKQAHAHSK; SEQ ID NO:892); Transportan (GWTLNSAGYLLGKINLKALAALAKKIL; SEQ ID NO:893); MAP17 (QLALQLALQALQAALQLA; SEQ ID NO:894); VT5 (DPKGDPKGVTVTVTVTVTGKGDPKPD; SEQ ID NO:895); Bac7 (RRIRPRPPRLPRPRPRPLPFPRPG; SEQ ID NO:896); (PPR)n ((PPR)3, (PPR)4, (PPR)5, (PPR)6); gH625 (HGLASTLTRWAHYNALIRAF; SEQ ID NO:897); and GALA (WEAALAEALAEALAEHLAEALAEALEALAA; SEQ ID NO:898). Additional examples of CPPs can be found in Singh et al. (e.g. Singh et al, Drug Deliv (2018) 25(1):2005-2015)), which is hereby incorporated by reference in its entirety.

### Modified RNA-Binding Protein

The subject modified RNA-binding protein (RBP) includes one or more endosomolytic peptides, which are covalently linked to the RBP directly or via a linker or tether. As disclosed herein, a modified RBP present in a system of the present disclosure includes an RBP that comprises a segment (e.g., a domain) that has affinity for RNA. It will be understood that the affinity for RNA need not have sequence specificity. An ELP conjugated to an RBP includes a segment, or an amino acid sequence, capable of promoting transit of co-localized macromolecular cargo from the endosome to the cytosol.

As disclosed herein, the modified RBP includes one or more ELPs covalently bound either directly or indirectly via a linker to the RBP of interest. It will be understood that conjugation of the ELP to the RBP may be achieved by any convenient methodology. The subject RBP is chosen to give this system the versatility to co-localize a precise number of ELPs to the cargo RNA complex. Through the variable number of RBP binding sites in the cargo RNA as well as the variable number of ELPs conjugated to the RBP itself, the system permits control of the number of endosomal escape-promoting polypeptides being co-localized with the cargo RNA.

### RNA-Binding Proteins

As disclosed herein, a modified RBP present in a system of the present disclosure includes an RBP that comprises a segment (e.g., a domain) that has affinity for RNA. It will be understood that the affinity for RNA need not have sequence specificity.

In some cases, the RBP present in a modified RBP of a system of the present disclosure has a length of from about 25 amino acids to about 250 amino acids; e.g., from about 25 amino acids (aa) to about 50 aa, from about 50 aa to about 75 aa, from about 75 aa to about 100 aa, from about 100 aa to about 125 aa, from about 125 aa to about 150 aa, from about 150 aa to about 175 aa, from about 175 aa to about 200 aa, from about 200 aa to about 225 aa, or from about 225 aa to about 250 aa. In some cases, the RBP present in a modified RBP of a system of the present disclosure has a length of from about 75 amino acids to about 125 amino acids. In some cases, the RBP present in a modified RBP of a system of the present disclosure has a length of from about 100 amino acids to about 150 amino acids. In some cases, the RBP present in a modified RBP of a system of the present disclosure has a length of from about 95 amino acids to about 105 amino acids. In certain cases, the RBP has a length of about 100 amino acids. In some cases, the RBP has a length of 98 amino acids, 99 amino acids, 100 amino acids, 101 amino acids, or 102 amino acids.

In some cases, the RBP comprises an amino acid sequence having at least 85%, at least 90%, at least 95%, at least 98%, at least 99%, or 100%, amino acid sequence identity to the amino acid sequence of an RBP selected from an MS2 coat polypeptide (see e.g., Golmohammade et al. J Mol Biol (1993) 234(3):620-639), a U1A snRNP polypeptide (e.g. UniProtKB/Swiss-Prot: P09012.3), a U2B polypeptide (e.g. NCBI Ref Seq NP_003083.1), a PP7 viral coat polypeptide (see e.g., Dhaese et al, Biochem Biophys Res Commun (1980) 94(4): 1394-1400), a poly(A)-binding protein (PABP) (see e.g., Eliseeva et al, Biochemistry (Moscow) 78(13):1377-1391), a stem-loop binding polypeptide, a boxB polypeptide (see e.g., Das Annu Rev Biochem (1993) 62:893-930), a Csy4 polypeptide (see e.g., Haurwitz et al. Science (2010) 329:1355-1358), a HuA polypeptide, a HuB polypeptide, a HuC polypeptide, a HuD polypeptide (see e.g., Hinman and Lou, Cell Mol Life Sci (208) 65(20):3168-81), an hnRNP polypeptide (see e.g. Geuens et al, Hum Genet (2016) 135(8):851-67), a CArG polypeptide (see e.g., Penalver-Mellado et al. Mol Microbiol 61:910-926), a NusA polypeptide (see e.g., Mooney et al, Mol Cell (2009) 33:97-108), a KH-type polypeptide (e.g., a Nova-2 KH3 polypeptide, see e.g., Siomi et al, Nucleic Acids Res (1993) 21:1193-1198)), or an eIF4A polypeptide (see e.g., NM_001204510.2, UniProtKB/Swiss-Prot P60842), which are hereby incorporated by reference in their entirety. Particular examples of RBPs for use in the present system include but are not limited to, the N-terminal domain of human U1A, an MS2 protein, and a PP7 viral coat protein, including variants or derivatives thereof.

In some cases, the RNA binding protein is selected from the RNA Recognition Motif (RRM) family of cellular proteins involved in pre-messenger RNA processing. One example of such a protein is the U1A snRNP protein. More than 200 members of the RRM superfamily have been reported, the majority of which are ubiquitously expressed and conserved in phylogeny (Query et al, Cell (1989) 57: 89-101; Kenan et al, Trends Biochem. Sci. (1991) 16: 214-220). Most are known to have binding specificity for polyadenylate mRNA or small nuclear ribonucleic acids (e.g. U1, U2, etc.) transfer RNAs, 5S or 7S RNAs. They include but are not limited to hnRNP proteins (A, B, C, D, E, F, G, H, I, K, L), RRM proteins CArG, DT-7, PTB, K1, K2, K3, HuD, HUC, rbp9, elF4B, sxl, tra-2, AUBF, AUF, 32KD protein, ASF/SF2, U2AF, SC35, and other hnRNP proteins.

In some cases, the RBP is a UA1 polypeptide comprising amino acid sequence having at least 85%, at least 90%, at least 95%, at least 98%, at least 99%, or 100%, amino acid sequence identity to one of the amino acid sequences depicted in FIG. 1. The UA1 polypeptide can be modified to include 1, 2, 3, 4, 5, or more than 5, cysteine residues.

In certain cases, the RBP includes an underlying sequence (e.g., a consensus sequence of fixed amino acid residues) having 60% or more amino acid sequence identity, such as 70% or more, 80% or more, 85% or more, 90% or more, 95% or more or 98% or more amino acid sequence identity to the corresponding amino acid sequence of the native parent protein sequence. A subject RBP sequence may include 1 or more, such as 2 or more, 3 or more, 4 or more, 5 or more, 10 or more, 15 or more, or even 20 or more additional amino acids compared to a native parent protein sequence, e.g., in the form on a N-terminal or C-terminal extension sequence or in the form of an insertion mutation. In some cases, 30 or less additional amino acids, such as 1-20 residues, 2-10 residues, or even 2-5 additional peptidic residues are included in the parent RBP native parent protein sequence. Alternatively, a subject RBP sequence may include fewer amino acids compared to a native parent protein sequence motif, such as 1, 2, 3, 4, 5, 6, 7, 8, 9 or 10, or even fewer residues, e.g., by having deletions at the N-terminus and/or C-terminus, truncations, or modifications at locations in the sequence that do not adversely affect the structural motif.

The RBP may include mutations at various positions of the native parent protein sequence, e.g., variant amino acids at certain positions within the protein scaffold. When a RBP arises from amino acid mutations at various positions within the native parent protein sequence, the amino acids at those positions are referred to as “variant amino acids.” The underlying parent sequence includes those residues that are “fixed amino acids” (e.g., non-variant amino acids). Such variant amino acids may confer on the resulting RBPs different functions, such as specific binding to a target endosomolytic peptide, or increased stability relative to the parent protein sequence.

Any convenient locations of the RBP native parent protein sequence of interest may be selected for any convenient number of mutations (e.g., 1, 2, 3, 4, 5 or more mutations). In some cases, five or more mutations may be introduced at any convenient five or more variant amino acid locations of the parent protein. In other cases, five or less mutations may be introduced at any convenient five or less variant amino acid locations of the parent protein.

In some instances, the RBP native parent protein sequence includes five or more mutations, such as 6 or more, 7 or more, 8 or more, 9 or more, 10 or more, 11 or more, 12 or more, 13 or more, 14 or more, 15 or more, 16 or more mutations, e.g., at any convenient position in the parent RBP sequence. In some instances, the RBP native parent protein sequence includes five or less mutations, such as 4 or less, 3 or less, 2 or less, 1 or no mutations, e.g., at any convenient position in the parent RBP sequence.

The subject RBP may be any of any convenient length. In some cases, the subject RBP includes an amino acid sequence of between 45 and 200 residues, such as between, 45 and 180 residues, 45 and 160 residues, 45 and 140 residues, 45 and 120 residues, 45 and 100 residues, 45 and 80 residues, 45 and 60 residues, 60 and 200 residues, 60 and 180 residues, 60 and 160 residues, 60 and 140 residues, 60 and 120 residues, 60 and 100 residues, 60 and 80 residues, 80 and 200 residues, 80 and 180 residues, 80 and 160 residues, 80 and 140 residues, 80 and 120 residues, 80 and 110 residues, 80 and 100 residues, 90 and 200 residues, 90 and 150 residues, 90 and 110 residues. In some cases, the subject RBP includes an amino acid sequence of about 100 residues. It is understood that the number of residues comprised in each RBP may vary according to the underlying scaffold, extension sequences, mutations included, etc.

In one instance, the RNA binding protein (RBP) is the N-terminal domain of a human U1A protein, also referred to herein as “U1A”. The U1A construct comprises a sequence as follows:

(SEQ ID NO: 822)
MAVPETRPNHTIYINNLNEKIKKDELKKSLYAIFSQFGQILDILVSRSLK
MRGQAFVIFKEVSSATNALRSMQGFPFYDKPMRIQYAKTDSDIIAKMK

The RNA binding protein (RBP) may comprise a U1A protein as shown above (SEQ ID NO: 822) including mutations at various positions of the parent sequence, e.g., variant amino acids at certain positions within the U1A protein.

It will be understood that the RBP may comprise a U1A protein as shown above (SEQ ID NO: 822) or a fragment or modified sequence thereof, e.g., having 1 or more C or N terminal deletions or truncations, e.g. by 1-5 amino acid residues.

In one example, the modified RBP comprises a U1A protein of SEQ ID NO: 822 comprising 2 or more mutations, such as 3 or more, 4 or more, 5 or more, or even more. In some cases, the RBP comprises a modified U1A protein of SEQ ID NO: 822 comprising 5 or less mutations, such as 4 or less, 3 or less, 2 or less, 1 or no mutations. Any convenient locations of the RBP protein of interest may be selected for any convenient number of mutations (e.g., 1, 2, 3, 4, 5 or more mutations). In some cases, one or more mutation is a conservative amino acid substitution (e.g., as described herein).

In some embodiments of the U1A protein, the Y31 residue is substituted with a H residue (Y31H). In some embodiments of the U1A protein, the Q36 residue is substituted with an R residue (Q36R).

In some embodiments of the U1A protein, at least one S residue is substituted with a C residue. In some cases, two S residues are substituted with C residues. In other cases, three S residues are substituted with C residues. In some cases, S71 is substituted with a C residue (S71C). In some cases, S35 is substituted with a C residue (S35C). In some cases, S63 is substituted with a C residue (S63C). In certain cases, S35 and S71 are substituted with C residues (S35C and S71C). In some cases, S63 and S35 are substituted with C residues (S63C and S35C). In some cases, S63 and S71 are substituted with C residues (S63C and S71C). In some cases, S35, S63 and S71 are mutated to C residues (S35C, S63C and S71C). In some cases, the sites substituted with a C residue are on the opposite face of the U1A protein from the RNA-binding site.

In some embodiments of the modified U1A protein (e.g. a U1A protein that has been modified with a mutation and/or substitution as described herein), the Y, Q and S residues are substituted. In some cases, the Y31 residue is substituted with an H residue (Y31H), the Q36 residue is substituted with an R residue (Q36R). In some cases, one or more of amino acid positions 35, 63 and 71 are modified with one or more residue substitutions. In some cases, the modified U1A protein comprises Y31H, S35C and Q36R substitutions. In some cases, the modified U1A protein comprises Y31H, S35C, Q36R and S63C substitutions. In some cases, the modified U1A protein comprises Y31H, S35C, Q36R and S71C substitutions. In some cases, the modified U1A protein comprises Y31H, S35C, Q36R, S63C and S71C substitutions.

In some cases, the RBP is a U1A protein including three or more mutations located at three or more positions, e.g., as depicted in any of SEQ ID NOs: 823-825 (mutations underlined, see also FIG. 1):

(SEQ ID NO: 823)
MAVPETRPNHTIYINNLNEKIKKDELKKSLHAIFSRFGQILDILVSRSLK
MRGQAFVIFKEVSSATNALRCMQGFPFYDKPMRIQYAKTDSDIIAKMK
(SEQ ID NO: 824)
MAVPETRPNHTIYINNLNEKIKKDELKKSLHAIFCRFGQILDILVSRSLK
MRGQAFVIFKEVSSATNALRCMQGFPFYDKPMRIQYAKTDSDIIAKMK
(SEQ ID NO: 825)
MAVPETRPNHTIYINNLNEKIKKDELKKSLHAIFCRFGQILDILVSRSLK
MRGQAFVIFKEVCSATNALRCMQGFPFYDKPMRIQYAKTDSDIIAKMK

In some cases, the RBP is an MS2 coat protein. In some cases, an MS2 coat protein comprises an amino acid sequence having at least 85%, at least 90%, at least 95%, at least 98%, at least 99%, or 100%, amino acid sequence identity to the following MS2 coat protein amino acid sequence:

(SEQ ID NO: 849)
MASNFTQFVL VDNGGTGDVT VAPSNFANGV AEWISSNSRS
QAYKVTCSVR QSSAQNRKYT IKVEVPKVAT QTVGGVELPV
AAWRSYLNME LTIPIFATNS DCELIVKAMQ GLLKDGNPIP
SAIAANSGIY.

In one example, the modified RBP comprises a MS2 coat protein comprising 1 or more mutations (e g amino acid substitutions), such as 2 or more, 3 or more, 4 or more, 5 or more, or even more. Any convenient locations of the MS2 scaffolds of interest may be selected for any convenient number of mutations (e.g., 1, 2, 3, 4, 5 or more mutations). In some cases, amino acid residues in particular locations in the wildtype sequence of MS2 are substituted with cysteines, histidines and/or arginines.

In some cases, the RBP is a PP7 coat protein. In some cases, a PP7 coat protein comprises an amino acid sequence having at least 85%, at least 90%, at least 95%, at least 98%, at least 99%, or 100%, amino acid sequence identity to the following PP7 coat protein amino acid sequence:

(SEQ ID NO: 850)
SKTIVLSVGE ATRTLTEIQS TADRQIFEEK VGPLVGRLRL
TASLRQNGAK TAYRVNLKLD QADVVDCSTS VCGELPKVRY
TQVWSHDVTI VANSTEASRK SLYDLTKSLV VQATSEDLVV
NLVPLGR.

In one example, the modified RBP comprises a PP7 protein sequence motif comprising 2 or more mutations (e.g., amino acid substitutions), such as 3 or more, 4 or more, 5 or more, or even more. Any convenient locations of the PP7 scaffolds of interest may be selected for any convenient number of mutations (e.g., 1, 2, 3, 4, 5 or more mutations). In some embodiments, amino acids residues in particular locations in the wildtype sequence of PP7 are substituted with cysteines, histidines and/or arginines.

It will be understood that any of RBPs disclosed herein (e.g., a U1A protein, an MS2 protein, a PP7 protein, etc.) may include additional mutations, fragments and other modified variations thereof. A mutation in a parent sequence or fragment thereof may include a deletion, insertion, or substitution of an amino acid residue at any convenient position to produce a sequence that is distinct from the parent sequence or fragment, yet still retains affinity for RNA. In certain cases, the amino acid substitution is a conservative amino acid substitution (e.g., as described herein). In certain cases, a RBP is modified at the C and/or N terminus with a certain number of C or N terminal deletions, truncations or additional amino acid residues, e.g., such as the addition or deletion of 1 to 5 amino acid residues.

In some cases, the RBPs disclosed herein include a chemoselective functional group or a compatible functional group that is capable of conjugating to a chemoselective group or a compatible functional group or a of an ELP. In certain cases, the chemoselective or compatible functional groups for inclusion in the subject RBPs, include, but are not limited to: an azido group, an alkynyl group, a phosphine group, a cysteine residue, a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfones. In certain cases, the RBP comprises a defined number of cysteine residues (which may be introduced via site mutagenesis), of which one can chemically conjugate a defined number of ELPs. It will be understood that the cysteine residues or other group compatible for conjugation with an ELP may be at any convenient location within the RBP, e.g., on a sidechain of a residue, at the C-terminus or at the N-terminus. In certain cases, the cysteine residues or other group compatible for conjugation with an ELP is on the opposite face of the RBP from the RNA-binding site.

In some cases, a modified RBP comprises an ELP at the N-terminus of an RBP. In some cases, a modified RBP comprises an ELP at the C-terminus of an RBP. In some cases, a modified RBP comprises an ELP at the N-terminus of an RBP and at the C-terminus of an RBP. In some cases, a modified RBP comprises a single ELP at the N-terminus of an RBP. In some cases, a modified RBP comprises a single ELP at the C-terminus of an RBP. In some cases, a modified RBP comprises, in order from N-terminus to C-terminus: i) an ELP; and ii) an RBP. In some cases, a modified RBP comprises, in order from N-terminus to C-terminus: i) an ELP; ii) a linker peptide of from 1 to 10 amino acids in length; and iii) an RBP. In some cases, a modified RBP comprises, in order from N-terminus to C-terminus: i) an RBP; and ii) an ELP. In some cases, a modified RBP comprises, in order from N-terminus to C-terminus: i) an RBP; ii) a linker peptide of from 1 to 10 amino acids in length; and iii) an ELP.

In some cases, a modified RBP comprises one or more ELP(s) at the N-terminus of an RBP and one or more ELP(s) covalently linked at internal location(s) within the RBP protein sequence. In some cases, a modified RBP comprises one or more ELP(s) at the C-terminus of the RBP and one or more ELP(s) covalently linked at internal location(s) within the RBP protein sequence. In some cases, a modified RBP comprises, in order from N-terminus to C-terminus: i) an ELP; and ii) an RBP comprising an ELP linked at an internal location within the RBP. In some cases, a modified RBP comprises, in order from N-terminus to C-terminus: i) an ELP; ii) a linker peptide of from 1 to 10 amino acids in length; and iii) an RBP comprising a linker peptide of from 1 to 10 amino acids in length covalently linked to an ELP. In some cases, a modified RBP comprises, in order from N-terminus to C-terminus: i) an RBP comprising one or more ELPs covalently linked at internal location(s) within the RBP sequence; and ii) an ELP. In some cases, a modified RBP comprises, in order from N-terminus to C-terminus: i) an RBP comprising a linker peptide of from 1-10 amino acids in length covalently linked to an ELP and ii) an ELP. In any of the above embodiments comprising a U1A protein, the internal location for the attachment of the one or more ELP(s) may be chosen from positions 31, 35, 36, 63 and/or 71. In some cases, the RBP comprises additional modifications to add functionality and/or assist in protein purification. In some cases, the RBP comprises one or more nuclear localization sequences (NLS) to facilitate transport of the RBP into the nucleus. The RBP may comprise 1, 2, 3, 4, 5, 6, 7, 8, 9, 10 or more NLSs. In some cases, the RBP comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxyl-terminus, or a combination of these (e.g. one or more NLS at the amino-terminus and one or more NLS at the carboxyl terminus). Non-limiting examples of NLS sequences include the monopartite SV40 large T antigen NLS (PKKKRRV; SEQ ID NO:899) and the bipartite NLS from nucleoplasmin (KRPAATKKAGQAKKKK; SEQ ID NO:900). See e.g. (Lange et al, J Biol Chem (2007) 282(8):5101-5105), which is hereby incorporated by reference in its entirety. Other examples include the NLS from c-Myc (PAAKRVKLD (SEQ ID NO:901); or RQRRNELKRSP (SEQ ID NO:902)), EGL-13 (MSRRRKANPTKLSENAKKLAKEVEN; SEQ ID NO:903), and TUS protein (KLKIKRPVK; SEQ ID NO:904); the nRNPA1 M9 NLS (NQSSNFGPMKGGNFGGRSSGPYGGGGQYFAKPRNQGGY; SEQ ID NO:905); the IBB domain from importin-alpha (RMRIZFKNKGKDTAELRRRRVEVSVELRKAKKDFQILKRRNV; SEQ ID NO:906); the NLS sequences from the myoma T protein (VSRKRPRP (SEQ ID NO:907); and PPKKARED (SEQ ID NO:908)); the p53 protein sequence POPKKKPL (SEQ ID NO:916); the sequence SALIKKKKKMAP (SEQ ID NO:909) of mouse c-abl IV; the sequences DRLRR (SEQ ID No:910) and PKQKKRK (SEQ ID NO:911) of the influenza virus NS1); the sequence RKLKKKIKKL (SEQ ID NO:912) of the Hepatitis virus delta antigen; the sequence REKKKFLKRR (SEQ ID NO:913) of the mouse Mxl protein; the sequence KRKGDEVDGVDEVAKKKSKK (SEQ ID NO:914) of the human poly(ADP-ribose) polymerase; and the sequence RKCLQAGMNLEARKTKK (SEQ ID NO:915) of the steroid hormone receptors (human) glucocorticoid. See e.g. Ray et al., Bioconjug Chem (2015) 26 (6):1004-7, which is hereby incorporated by reference in its entirety. In some cases, the RBP comprises a peptide tag to assist in purification and modification of the protein. In some cases, the RBP comprises a HIS tag (e.g. a 6×His-tag, see Hochuli et al., Bio/Technology (1988) 6(11):1321-5), which is hereby incorporated by reference in its entirety. Other examples of peptide tags to assist purification include glutathione S-transferase followed by a protease cleavage site (e.g. GST fusion); maltose binding protein fusions (MBP fusion); calmodulin binding peptide (CBP fusion); intein-chitin binding domain (intein-CBD); streptavidin/biotin based tags; FLAG tags; reporter tags such as ®-galactosidase, alkaline phosphatase, and the like; small ubiquitin like modifier fusions, and PDZ domain-based tags. Non-limiting examples of peptide tags can be found in Kimple et al. (Curr Protoc Protein Sci (2013) 73, unit 9-9), which is hereby incorporated by reference in its entirety.

### Endosomolytic Peptides

As disclosed herein the ELPs include amino acid sequences capable of promoting transit of co-localized macromolecular cargo (e.g., a modified RBP complexed with a modified cargo RNA, as described herein) from the endosome (or other cellular compartment, e.g., a membrane-bound cellular compartment) to the cytosol. Any convenient ELP capable of promoting transit of co-localized macromolecular cargo from the endosome to the cytosol can be used in the present systems.

The subject ELPs promote endosomal escape following endocytosis and as such are capable of preventing the macromolecular cargo from being trafficked to the lysosome for rapid degradation. In some cases, the ELP is a cell penetrating peptide. In other cases, the ELP is not a cell penetrating peptide.

In some cases, the subject ELP is polyanionic, peptidomimetic or a peptide having a neutral or near-neutral charge at physiological pH, which shows pH-dependent membrane lytic activity and leads to endosome lysis or leakage. In certain cases, the endosomolytic peptide assumes its active conformation at endosomal pH. The “active” conformation is that conformation in which the endosomolytic component promotes lysis of the endosome and/or transport of the cargo RNA of the invention, or its components, from the endosome to the cytoplasm of the cell. See, e.g., Martin M. E. et al. Peptide-guided gene delivery, the AAPS Journal, 2007, 9(1), article 3. The term “activation”, as used herein when referring to an ELP, refers to protonation of the ELP molecule such that it is able to promote endosome lysis. For example, protonation will occur to an increasing percent as the local pH decreases such that endosomal lysis can occur at more neutral pHs and increase in efficiency as the pH decreases.

In some cases, the subject ELP has a length of from about 5 amino acids to about 60 amino acids, such as 5 to 10, 10 to 20, 20 to 30, 30 to 40, 40 to 50 or 50 to 60 amino acids. In some case, the subject ELP has a length of less than 60 amino acids, such as 50 or less, 40 or less, 35 or less, 30 or less, 25 or less, 20 or less, 15 or less, 10 or less. In certain cases, the ELP is from 20 to 30 amino acids, such as 20, 21, 22, 23, 24, 25, 26, 27, 28, 29 or 30 amino acids. In certain cases, the ELP has a length of from about 20 to 35 amino acids, such as 20, 21, 22, 23, 24, 25, 26, 27, 28, 29, 30, 31, 32, 33, 34, 35 amino acids. In some cases, the ELP has a length of about 20 amino acid residues. In other cases, the ELP has a length of 27 amino acid residues. In other cases, the ELP has a length of 28 amino acid residues. In some other cases, the ELP has a length of 33 amino acid residues.

In some cases, the ELPs are rich in histidine residues. In some cases, the ELP comprises from 20% to 70% histidine residues, such as 25% to 70%, 30% to 70%, 35% to 70%, 40% to 70%, 45% to 70%, 50% to 70%, 55% to 70%, 60% to 70% or 65 to 70% histidine residues. In other cases, the ELP comprises from 20% to 30%, 20% to 35%, 20% to 35%, 20% to 40%, 20% to 45%, 20% to 50%, 20% to 55%, 20% to 60% or 20% to 65%. In some cases, the ELP comprises 20% or more histidine residues, such as 25% or more, 30% or more, 40% or more, 45% or more, 55% or more, 60% or more, or 65% or more, or even more. In certain cases, the ELP comprises less than 70% histidine residues, such as 65% or less, 50% or less, 40% or less, 30% or less, or even less. In some cases, the ELP comprises 25% to 30% histidine residues. In some cases, the ELP comprises 60% to 70% histidine residues.

In some cases, the ELP comprises a histidine-rich peptide selected from, but not limited to, GLFHALLHLLHSLWHLLLHA (SEQ ID NO: 817), CHK₆HC (SEQ ID NO:826); H₅WYG (SEQ ID NO: 827), GLFHAIAHFIHGGWHGLIHGWYG (SEQ ID NO:828), (H2E)9: HHEHHEHHEHHEHHEHHEHHEHHEHHE (SEQ ID NO: 821); and variants, analogues or derivatives thereof.

In some cases, the ELP comprises a synthetic peptide. In some cases, the ELP comprises ppTG21: GLFHALLHLLHSLWHLLLHA (SEQ ID NO: 817), having the structure:

[chemical structure]

wherein the hydrogen atoms are omitted from the peptide structure for clarity of view. See Rittner, Karola, et al., New Basic Membrane-Destabilizing Peptides for Plasmid-Based Gene Delivery in Vitro and in Vivo, MOLECULAR THERAPY 5(2) 104-114 (2002).

In some cases, the ELP comprises 55% or less cationic amino acid residues, such as 50% or less, 45% or less, 40% or less, 30% or less, or even less. In other cases, the ELP comprises from 20% to 55% cationic residues, such as 20% to 25%, 20% to 30%, 20% to 35%, 20% to 40%, 20% to 45%, 20% to 50% cationic residues. In some cases, the ELP comprises 20% to 25% cationic residues. In some cases, the ELP comprises 30% to 40% cationic residues. In some cases, the ELP comprises 45% to 55% cationic residues.

In some cases, the ELP is a poly(histidine) peptide. For example, in some cases, the ELP is (His)ₙ where n is an integer from 1 to 10, e.g., where n is 1, 2, 3, 4, 5, 6, 7, 8, 9, or 10.

In some cases, the ELP comprises only leucine and histidine residues. For example, in some cases, the ELP comprises the amino acid sequence

(SEQ ID NO: 851)
LHHLLHHLLHHLHHLLHHLHHLLHHL.

In some cases, the ELP comprises E5-TAT (see e.g., Lee et al, Biochemistry (2010) 49(36):7854-7866): GLFEAIAEFIENGWEGLIEGWYGGRKKRRQ RRR (SEQ ID NO: 818), a variant, analog or derivative thereof.

In some cases, the ELP comprises Miniature Protein 5.3 (see e.g., Appelbaum, et al, Chem Biol (2012) 19(7):819-830): GPSQPTYPGDDAPVRDLIRFYRDLRRY (SEQ ID NO: 819), a variant, analog, or derivative thereof.

In some cases, the ELP comprises ZF5.3 (see Appelbaum, ibid): WYSCNVCGKAFVLSRHLNRHLRVHRRAT (SEQ ID NO: 820), a variant, analog or derivative thereof.

In some cases, the ELP suitable for use herein comprises a plurality of proton acceptor sites having pKa values between physiological and lysosomal pH. In some cases, the ELP suitable for use herein comprises a plurality of proton acceptor sites having pKa values within the range of 4 to 7. In some cases, the ELP may be modified with groups selected from imidazole-containing compounds, e.g., peptides comprising one or more histidine, histamine, vinylimidazole, or combinations thereof.

In some cases, the ELP comprises a scrambled peptide of any of the subject ELPs (e.g., as described herein), wherein a “scrambled peptide” will be understood as a random permutation of the original peptide sequence.

It will be understood that any of ELPs disclosed herein may include additional mutations, fragments and other modified variations thereof. A mutation in a parent sequence or fragment thereof may include a deletion, insertion, or substitution of an amino acid residue at any convenient position to produce a sequence that is distinct from the parent sequence or fragment, yet still retains its ability to promote endosomal escape following endocytosis. In some cases, an amino acid substitution is a conservative amino acid substitution (e.g., as described herein). In certain cases, a ELP is modified at the C and/or N terminal with a certain number of C or N terminal deletions, truncations or additional amino acid residues, e.g., such as the addition or deletion of 1 to 5 amino acid residues.

In some cases, the ELPs disclosed herein comprise a chemoselective reactive functional group that is capable of conjugating to a compatible functional group of an RBP. In certain cases, the chemoselective reactive functional groups for inclusion in the subject peptidic compounds, include, but are not limited to: an azido group, an alkynyl group, a phosphine group, a cysteine residue, a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfones. In some cases, the chemoselective reactive functional group is a cysteine residue. In certain cases, the chemoselective reactive functional group is a cysteine residue which has been further modified with a functional group capable of facilitating a disulfide exchange reaction e.g. a pyridyl disulfide group.

In some cases, the ELP is a synthetically modified ppTG21 peptide bearing a pyridyl disulfide leaving group to facilitate conjugation, e.g., as described by the sequence GLFHALLHLLHSLWHLLLHA (C) [pyridyl disulfide] (SEQ ID NO: 817), wherein the ppTG21 peptide includes a C-terminal cysteine residue (C) to facilitate thiol-based conjugation to a cysteine-bearing U1A RBP.

### Fusion Polypeptides

As noted above, any of the ELPs described herein can be conjugated to RBP of interest by any convenient conjugation chemistry, to generate a modified RBP. Alternatively, a modified RBP can be a fusion polypeptide.

In some cases, a modified RBP is a fusion polypeptide comprising an ELP at the N-terminus of an RBP. In some cases, a modified RBP is a fusion polypeptide comprising an ELP at the C-terminus of an RBP. In some cases, a modified RBP is a fusion polypeptide comprising an ELP at the N-terminus of an RBP and at the C-terminus of an RBP. In some cases, a modified RBP is a fusion polypeptide comprising an ELP located at an internal location of an RBP. In some cases, a modified RBP is a fusion polypeptide comprising an ELP at the N-terminus, the C-terminus, and/or an ELP located at an internal location of an RBP. In some cases, a modified RBP is a fusion polypeptide comprising a single ELP at the N-terminus of an RBP. In some cases, a modified RBP is a fusion polypeptide comprising a single ELP at the C-terminus of an RBP. In some cases, a modified RBP is a fusion polypeptide comprising, in order from N-terminus to C-terminus: i) an ELP; and ii) an RBP. In some cases, a modified RBP is a fusion polypeptide comprising, in order from N-terminus to C-terminus: i) an ELP; ii) a linker peptide of from 1 to 10 amino acids in length; and iii) an RBP. In some cases, a modified RBP is a fusion polypeptide comprising, in order from N-terminus to C-terminus: i) an RBP; and ii) an ELP. In some cases, a modified RBP is a fusion polypeptide comprising, in order from N-terminus to C-terminus: i) an RBP; ii) a linker peptide of from 1 to 10 amino acids in length; and iii) an ELP.

### Conjugation

Any of the ELPs described herein can be conjugated to an RNA-binding protein (RBP) of interest by any convenient conjugation chemistry. In one example, the ELP includes a chemoselective functional group that is capable of conjugating to a compatible functional group in an RBP of interest, or vice versa. In certain cases, the RBP of interest can include a precise number of compatible functional groups, so as to conjugate a precise number of ELPs to obtain a modified ELP of interest. In certain cases, the RBP includes 1 or more compatible functional groups, such as 2, 3, 4, 5, 6, 7, 8, 9 or 10. In some cases, the RBP includes 10 or fewer compatible functional groups, such as 9, 8, 7, 6, 5, 4, 3, 2, 1. In certain cases, the RBP of interest includes 3 compatible functional groups. In some cases, the RBP of interest includes 2 compatible functional groups. In other cases, the RBP of interest includes 1 compatible functional group. It will be understood that an RBP of interest may include, or be modified to include, any convenient number of compatible functional groups for conjugation to a subject ELP.

In some cases, the modified RBP is described by the formula:

X-(L-Z)n

wherein, X is a RBP (e.g. as described herein), L is an optional linking group, Z is an ELP of interest and n is an integer from 1 to 10. Z, or L if present, is attached to X at any convenient location (e.g., via a sidechain of a residue, the N-terminus or the C-terminus).

In some cases, the ELP peptide (Z) includes a chemoselective group, e.g. at its N-terminus or its C-terminus. In some instances, the chemoselective group is covalently attached via the alpha-amino group of the N-terminal residue, or is covalently attached to the alpha-carboxyl acid group of the C-terminal residue. In other instances, the chemoselective group is attached to the ELP via a sidechain group of a residue. In certain cases, the chemoselective group is a cysteine residue. In certain cases, the cysteine residue has been modified with a functional group capable of facilitating a disulfide exchange reaction with a cysteine residue in the RBP of interest e.g., modified to include a pyridyl disulfide group.

In some cases, the RBP (X) includes 1 to 10 compatible functional groups for ELP attachment, such as 1, 2, 3, 4, 5, 6, 7, 8, 9 or 10 compatible groups. In some cases, the RBP (X) includes 1 to 5 compatible functional groups for ELP attachment, such as 1, 2, 3, 4 or 5 compatible groups. In some cases, the RBP includes 1 compatible functional group. In some cases, the RBP includes 2 compatible functional groups. In some cases, the RBP includes 3 compatible functional groups. In some cases, the RBP includes 4 compatible functional groups. In some cases, the RBP includes 5 compatible functional groups. In some cases, all compatible functional groups are on a sidechain of a residue. In some cases, at least one compatible functional group is at the C-terminus of the protein. In some cases, at least one compatible functional group is at the N-terminus of the protein. In certain cases, a compatible functional group on the RBP, includes, but is not limited to: an azido group, an alkynyl group, a phosphine group, a cysteine residue, a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfones.

In certain cases, the compatible functional group on the RBP is a cysteine residue. In certain cases, the RBP comprises one or more cysteine residues as the compatible functional group. Generally speaking, a single RBP may contain a precise number of cysteine residues (which may be introduced via site mutagenesis), of which one can chemically conjugate a precise number of ELPs. In one example, the ELPs contain cysteine residues modified with a pyridyl disulfide group to facilitate disulfide exchange reaction with the compatible cysteine residues of the RBPs.

It will be understood that conjugation of the ELPs to an RBP of interest can be readily achieved by a wide variety of commercially reactive compounds cross-linkers or via novel cross-linkers containing commonly used reactive groups such as, modified cysteine residues for disulfide exchange, N-hydroxysuccinimide (NHS)-esters/sulfonyl chlorides/isothiocyanates, maleimides/bromoacetimides and “click chemistry” involving a terminal alkyne or activated alkyne analogue (e.g. cyclooctyne) and an azide or a nitrone.

The term “click chemistry” is used to describe any facile reaction that occurs in high yields, under mild conditions, and in the presence of diverse functional groups, but it is most commonly used to refer to a [3+2] azide-alkyne cycloaddition reaction (see e.g., Musumeci et al, Curr Med Chem (2015) 22(17):2022-50). Such reactions are generally catalyzed by CuI, but may also be copper free (e.g., with a strained alkyne, such as a cyclooctyne), and proceed in the presence of functional groups typically encountered in biological molecules. In some cases, an unnatural sidechain or terminal group may be introduced into the RBP, wherein the sidechain or terminal group is modified to comprise an alkyne or azide. Accordingly, the ELP of interest may be modified to include a compatible alkyne or azide to facilitate the click reaction.

In some cases, one or more linkers are present between the RBP and one or more of the ELPs. In some cases, the linker is between the ELP and a chemoselective group capable of conjugating to an RBP of interest. In some cases, the linker is between the RBP and a compatible functional group capable of conjugating an ELP of interest. The linker may be present at any convenient location that facilitates conjugation between the RBP and ELP of interest. The linker may be cleavable or non-cleavable.

### Cleavable Linkers

In some cases, the RBP of the modified RBP is linked to the ELP via a cleavable linker. In some cases, the linker is cleavable under intracellular conditions, such that the cleavage of the linker releases the ELP from the modified RBP in the intracellular environment. In some cases, the linker is cleavable by a cleaving agent that is present in the intracellular environment (e.g. within a lysosome or endosome or caveolus). One example of a cleavable linker is an enzymatically cleaved linker i.e., a peptidyl linker that is cleaved by an intracellular peptidase or protease enzyme, including but not limited to, a lysosomal or endosomal protease. In some cases, the peptidyl linker is at least two amino acids long or at least three amino acids long. Enzymatic cleaving agents include cathepsins B and D and plasmin, all of which are known to hydrolyze dipeptide drug derivatives resulting in the release of active drug inside the target cells (see Dubowchik, Gene M. et al., Cathepsin B-Labile Dipeptide Linkers for Lysosomal Release of Doxorubicin, Bioconjugate Chem. 2002, 13, 855-869). Such linkers include peptides and dipeptides including those described in the above publications which are incorporated herein by reference in their entireties for all purposes.

Peptide-based cleavable linking groups are peptide bonds formed between amino acids to yield oligopeptides (e.g., dipeptides, tripeptides etc.) and polypeptides. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and R^{B} are the R groups of the two adjacent amino acids.

Other cleavable linkers may be cleaved by nucleophilic/basic reagents, reducing reagents, photo-irradiation, and electrophilic/acidic reagents. See, e.g., Leriche, Geoffray, et al., Cleavable Linkers in Chemical Biology, Bioorganic & Medicinal Chemistry 20 (2012) 571-582.

As described herein, a cleavable linking group is one which is sufficiently stable outside the cell, but which upon entry into a target cell is cleaved to release the two parts the linker is holding together. In one instance, the cleavable linking group is cleaved at least 10 times or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

In some cases, the cleavable linker which links the ELP to the RBPs is an acid-labile linking group. Upon entering the endosomal compartment, the acid-labile linking group is hydrolyzed, thereby releasing the ELP from the RBPs. In some cases, the cleavable linker which links the ELP from the RBPs is cleaved under reducing conditions upon entering the endosomal compartment. It is understood that any convenient cleavable linker capable of being cleaved to release the ELP once the cargo is inside the endosomal compartment, can be utilized in the subject systems and methods. The released ELP triggers endosomal disruption, allowing release of the cargo RNA into the cytoplasm.

More generally, the subject cleavable linking groups may be susceptible to cleavage agents, e.g., pH, redox potential or the presence of degradative molecules. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

In certain cases, the cleavable linker between the ELP and the modified RBPs hydrolyzes at acidic pH (e.g. the acidic conditions within the endosomal compartment). As used herein, the expression “acidic pH” means a pH of 6.0 or less (e.g., less than about 6.0, less than about 5.5, less than about 5.0, etc.). The expression “acidic pH” includes pH values of about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, 4.9, 4.85, 4.80, 4.75, 4.7, 4.65, 4.6, 4.55, 4.5 or less. In certain cases, the cleavable linker is cleaved rapidly at a pH of about 5.0, but slowly at a pH of about 7.4. Accordingly, any convenient cleavable linker which hydrolyzes under acidic conditions to release the ELP from the RBP (e.g. as described herein) may find use in the present invention. Examples of acid cleavable linking groups include but are not limited to hydrazones, esters, and esters of amino acids. Acid cleavable groups can have the general formula-C(═N—)N—, —C(O)O—, or —OC(O)—.

In general, the pH of human serum is 7.4, while the average intracellular pH is slightly lower, ranging from about 7.1-7.3. Endosomes have a more acidic pH, e.g., in the range of about 5.5-6.0, and lysosomes have an even more acidic pH at around 5.0. Some linkers will have a cleavable linking group that is cleaved at a preferred pH.

Some other linkers can include a cleavable linking group that is cleavable by a particular enzyme. Ester-based cleavable linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. In some cases, the carbon attached to the oxygen of the ester (the alkoxy group) is an aryl group, substituted alkyl group, or tertiary alkyl group such as dimethyl pentyl or t-butyl.

In certain instances, the cleavable linker between the ELP and the modified RBPs is cleaved under reducing conditions. In certain instances, the subject endosomal disruptor is cleaved by a reducing agent at acidic pH (e.g. the acidic conditions within the endosomal compartment). In certain cases, the cleavable linker is cleaved rapidly by glutathione reduction at the acidic pH of the endosomal compartment. Accordingly, any convenient cleavable linker that is capable of cleavage under reducing conditions may find use in the present invention. n certain instances, the cleavable linker is a disulfide. However, it will be understood that any convenient cleavable linker that is capable of cleavage within the endosomal compartment can be utilized in the subject endosomal disruptors.

In one instance, the cleavable linkers include redox cleavable linkers, such as a disulfide group (—S—S—) and phosphate cleavable linkers, such as, e.g., —O—P(O)(OR)—O—, —O—P(S)(OR)—O—, —O—P(S)(SR)—O—, —S—P(OXOR)—O—, —O—P(O)(OR)—S—, —S—P(O)(OR)—S—, —O—P(S)(OR)—S—, —S—P(S)OR)—O—, —O—P(O)(R)—, —O—P(S)R)—O—, —S—P(O)(R)—O—, —S—P(S)(R)—O—, —S—P(O)(R)—S—, —OP(S)(R)—S—, wherein R is hydrogen or alkyl.

In one instance, the redox cleavable linking group is a disulfide linking group (—S—S—). To determine if a candidate cleavable linking group is a suitable “reductively cleavable linking group,” any of a variety of well-known methods can be used. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. As an example, candidate compounds are cleaved by at most 10% in the blood. In some cases, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

In some cases, the cleavable linker is phosphate-based. The phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Suitable phosphate-based linking groups include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. For example, in some cases, the phosphate-based linking group is —O—P(O)(OH)—O—.

In some cases, the cleavable linker between the endosomolytic peptide and the modified RBPs cleaves within a timescale of 1 to 20 minutes at acidic pH. In some cases, the cleavable linker cleaves in less than 20 minutes, such as 15 minutes or less, 10 minutes or less, 5 minutes or less, 3 minutes or less. In certain cases, the cleavable linker is cleaved in a timescale from 10 to 15 minutes or less, such as 5 to 10 minutes or less. In some cases, the cleavable linker of the subject endosomal disruptors has a half-life of 2.5 minutes or less at a pH of about 5.

By contrast, at neutral pH, the cleavable linkers between the ELP and the modified RBPs may be stable for several hours.

In some cases, the subject released ELPs are metabolically stable (e.g., remain substantially intact in vivo during the half-life of the peptide). In certain cases, the peptides have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more. In some cases, the cleavable linker between the ELP and the modified RBPs has a half-life of greater than 4 hours at a pH of about 7.4.

As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In general, the suitability of a candidate cleavable linking group can be evaluated by testing the ability of a degradative agent (or condition) to cleave the candidate linking group. The candidate cleavable linking group can also be tested for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals.

### Cargo RNA

Suitable cargo RNA includes any convenient RNA sequence that has been modified to include a segment (stretch of nucleotides) that binds to the RBP portion of a modified RBP present in a system of the present disclosure. In some cases, the cargo RNA is a guide RNA. In some cases, the cargo RNA is a guide RNA that comprises a tracrRNA, e.g., a stretch of nucleotides that binds to an RNA-guided CRISRP/Cas effector polypeptide. Where the cargo RNA is a guide RNA, in some cases, the guide RNA is a single-molecule guide RNA (“single-guide RNA”). In some cases, the cargo RNA is a short interfering RNA (siRNA), a short hairpin RNA (shRNA), a ribozyme, an mRNA, a microRNA (miRNA), a small temporal RNA (stRNA), an antisense RNA, a small RNA-induced gene activation (RNAa), a small activating RNA (saRNA), a small nuclear RNA (snRNA), a small nucleolar RNA (snoRNA), SmY RNA, a small Cajal body-specific RNA (scaRNA), spliced leader RNA (SL RNA), cis-natural antisense transcript RNA (cis-NAT), long non-coding RNA (lncRNA), piwi-interacting RNA (piRNA), trans-acting RNA (tasiRNA), repeat associated RNA (rasiRNA), a telomerase RNA component (TERC), and the like. In some cases, the cargo RNA comprises a nucleotide sequence that encodes a polypeptide. In some cases, the cargo RNA does not encode a polypeptide. In some cases, the cargo RNA is a telomerase RNA component (TERC). In some cases, the RNA is an lncRNA.

### RBP Binding Sites

As noted above, a system of the present disclosure comprises a modified cargo RNA complexed to a modified RBP, where the modified cargo RNA comprises a cargo RNA modified to include one or more RBP binding sites that are bound by the RBP present in the modified RBP.

In some cases, a modified cargo RNA comprises a single RBP binding site. In some cases, a modified cargo RNA comprises 2 RBP binding sites. In some cases, a modified cargo RNA comprises 3 RBP binding sites. In some cases, a modified cargo RNA comprises 4 RBP binding sites. In some cases, a modified cargo RNA comprises 5 RBP binding sites.

In some cases, the RBP binding site is a contiguous stretch of from 4 nucleotides to 10 nucleotides, e.g., a contiguous stretch of 4 nucleotides (nt), 5 nt, 6 nt, 7 nt, 8 nt, 9 nt, or 10 nt.

Binding sites bound by a given RBP are known in the art. See, e.g., Laird-Offinga and Belasco (1995) Proc Natl Acad Sci USA 92: 11859-63; Hall and Stump (1992) Nucl. Acids Res. 20:4283; Oubridge et al. (1994) Nature 372:432.

For example, U1A recognizes the sequence AUUGCAC (SEQ ID NO:852). The AUUGCAC (SEQ ID NO:852) sequence may be flanked on the 5′ and 3′ ends by a stretch of nucleotides that form a base-paired region important to maintain a stem-loop structure. A U2B polypeptide (e.g., a U2B N-terminal fragment) also can bind the sequence AUUGCAC (SEQ ID NO:852). For example, a U2B polypeptide can bind the nucleotide sequence: CCUGGUAUUGCAGUACCUCCAGGU (SEQ ID NO:853), where the nucleotide sequences flanking AUUGCAG form the stem of a stem-loop structure. As another example, an MS2 coat protein recognizes a stem-loop structure comprising the sequence 5′-AAACAUGAGGAUUACCCAUGUCG-3′ (SEQ ID NO:854), or 5′-AAACAUGAGGAUCACCCAUGUCG-3′ (SEQ ID NO:855), where the AUUA (SEQ ID NO:856) or AUCA (SEQ ID NO:857) portion of the sequence is recognized with sequence specificity, and the remainder of the sequence is recognized with structure specificity. As another example, a PP7 viral coat protein recognizes the following nucleotide sequence GGCACAGAAGAAGAUAUGGCUUCGUGCC (SEQ ID NO:858), where sequence-specific recognition takes place at the underlined nucleotides; the remainder of the structure is recognized in a structure-specific manner. As another example, in PABP, the N-terminal 2 RNA recognition motifs recognized adenine-rich sequences, e.g., of at least 6 nucleotides in length (e.g., AAAAAA (SEQ ID NO:860)) (see, e.g., Deo et al. (1999) Cell 98:835). As another example, a boxB polypeptide (e.g., a polypeptide comprising NAKTRRHERRRKLAIERDT (SEQ ID NO:859)) can recognize the sequence GCCCUGAAAAAGGGC (SEQ ID NO:861). As another example, Csy4 recognizes the sequence ACUGCCGUAUAGGCAGC (SEQ ID NO:862). RBPs of the Hu family of RBPs include N-terminal domains that bind to AU-rich regions of ssRNAs; e.g., UUUUAUUUU (SEQ ID NO:863), UAUUUAUUUA (SEQ ID NO:864), and the like. HK-type RBPs, such as Nova-2 HK3 (residues 406-492) recognize the sequence GAGGACCUAGAUCACCCCUC (SEQ ID NO:865). A NusA polypeptide (e.g., residues 184-333) can recognize the sequence GAACUCAAUAG (SEQ ID NO:866).

As non-limiting examples, a single-guide RNA (sgRNA), modified to include a U1A binding site, can comprise a nucleotide sequence as follows (where: i) “[spacer]” refers to the approximately 20-nucleotide region that hybridizes to a target nucleic acid; ii) underlined nucleotides represent the sgRNA scaffold; iii) bold and double underlined nucleotides represent a based-paired region that provides for maintenance of stem-loop structure, but that is not recognized in a sequence-specific manner; v) italicized and underlined nucleotides (AUUGCAC; SEQ ID NO:852) represent the single-stranded region of RNA recognized by U1A in a sequence-specific manner; the downstream “UCC” sequence is essentially a single-stranded RNA linker that promotes favorable binding geometry of the loop and v) italicized nucleotides represent nucleotides (GCGAGGCUAAGU (SEQ ID NO:867); ACAGCACAAGCCCGCU (SEQ ID NO:868); and AGCAGGGAACUCGC (SEQ ID NO:869)) introduced to form a three-way junction):

“a”)
(SEQ ID NO: 870)
[spacer]GUUUAAGAGCUAU [symbol] AUUGCACUCC [symbol] GAUCCAUAGCAAGU
UUAAAUAAGGCUAGUCCGUUAUCAACUUGAAAAAGUGGCACCGAGUCGGU
GCUUUUUUU;
“b”)
(SEQ ID NO: 871)
[spacer]GUUUAAGAGCUAUGCUGGAAACAGCAUAGCAAGUUUAAAUAA
GGCUAGUCCGUUAUCAACUU [symbol] GCAAUCCAUUGCACUCC [symbol] AAGUGG
CACCGAGUCGGUGCUUUUUUU;
“c”)
(SEQ ID NO: 872)
[spacer]GUUUAAGAGCUAUGCUGGAAACAGCAUAGCAAGUUUAAAUAA
GGCUAGUCCGUUAUCAACUUGAAAAAGUGGCACCGAGUCGGUGCAUCAUC [symbol]
AUUGCACUCC [symbol] UUUUUUU;
“aa”)
(SEQ ID NO: 873)
[spacer]GUUUAAGAGCUAUGCGAGGCUAAGU [symbol] AUUGCACUCC [symbol] A
CAGCACAAGCCCGCU [symbol] UUGCACUCC [symbol] AGCAGGGAACUCGCAUAGC
AAGUUUAAAUAAGGCUAGUCCGUUAUCAACUUGAAAAAGUGGCACCGAGU
CGGUGCUUUUUUU;
and
bb)
(SEQ ID NO: 874)
[spacer]GUUUAAGAGCUAUGCUGGAAACAGCAUAGCAAGUUUAAAUAA
GGCUAGUCCGUUAUCAACUUGCGAGGCUAAGU [symbol] AUUGCACUCC [symbol] AC
AGCACAAGCCCGCU [symbol] AUUGCACUCC [symbol] [symbol] AGCAGGGAACUCGCAAGUGG
CACCGAGUCGGUGCUUUUUUU.
“bub”)
(SEQ ID NO: 923)
[spacer]GUUUAAGAGCUAUGCUGGAAACAGCAUAGCAAGUUUAAAUAA
GGCUAGUCCGUUAUCAACUUGUCGAGGCUAAGUCGCAUUGCACUCCGCGA
CAGCACAAGCCCGCUGCCAUUGCACUCCGGCAGCAGGGAACUCGUCAAGU
GGCACCGAGUCGGUGCUUUUUUU.

### Guide RNA

In some cases, the cargo RNA is a guide RNA. Guide RNAs suitable for inclusion in a system of the present disclosure include: i) a targeting segment comprising a nucleotide sequence that hybridizes to a nucleotide sequence in a target nucleic acid; and ii) an activation segment comprising a nucleotide sequence that binds to an activates an RNA-guided CRISPR/Cas effector polypeptide. In some cases, the RNA-guided CRISPR/Cas effector polypeptide is a class 2 CRISPR/Cas effector polypeptide. In certain cases, the class 2 CRISPR/Cas effector polypeptide is a type II CRISPR/Cas effector polypeptide. In certain cases, the class 2 CRISPR/Cas effector polypeptide is a Cas9 protein and the corresponding CRISPR/Cas guide RNA is a Cas9 guide RNA. In certain other cases, the class 2 CRISPR/Cas effector polypeptide is a type V or type VI CRISPR/Cas effector polypeptide. In some cases, the class 2 CRISPR/Cas effector polypeptide is a Cpf1 protein, a C2c1 protein, a C2c3 protein or a C2c2 protein. In some cases, the class 2 CRISPR/Cas effector polypeptide is a Cas12 enzyme. In other instances, the class 2 CRISPR/Cas effector enzyme is a Cas13 enzyme. In yet other cases, the class 2 CRISPR/Cas effector polypeptide is a base editor. Non-limiting examples of base editors can be found in Rees & Liu et al. (Rees & Liu, Nat Rev Genet. 2018 December; 19(12):770-788. doi: 10.1038/s41576-018-0059-1), which is hereby incorporated by reference in its entirety. In some cases, the class 2 CRISPR/Cas effector polypeptide is a CRISPR-mediated activator (CRISPRa). In some cases, the class 2 CRISPR/Cas effector polypeptide is a CRISPR interferer (CRISPRi). Non-limiting examples of CRISPRa and CRISPRi can be found in Dominguez et al. (Dominguez et al. Nat Rev Mol Cell Biol. (2016)17:5-15. DOI: 10.1038/nrm.2015.2), which is hereby incorporated by reference in its entirety.

A nucleic acid molecule that binds to a Cas9 protein and targets the complex to a specific location within a target nucleic acid is referred to herein as a “Cas9 guide RNA.”

A Cas9 guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule.

The first segment (targeting segment) of a Cas9 guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a Cas9 polypeptide. The protein-binding segment of a subject Cas9 guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the Cas9 guide RNA (the guide sequence of the Cas9 guide RNA) and the target nucleic acid.

A Cas9 guide RNA and a Cas9 protein form a complex (e.g., bind via non-covalent interactions). The Cas9 guide RNA provides target specificity to the complex by including a targeting segment, which includes a guide sequence (a nucleotide sequence that is complementary to a sequence of a target nucleic acid). The Cas9 protein of the complex provides the site-specific activity (e.g., cleavage activity or an activity provided by the Cas9 protein when the Cas9 protein is a Cas9 fusion polypeptide, i.e., has a fusion partner). In other words, the Cas9 protein is guided to a target nucleic acid sequence (e.g. a target sequence in a chromosomal nucleic acid, e.g., a chromosome; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the Cas9 guide RNA.

The “guide sequence” also referred to as the “targeting sequence” of a Cas9 guide RNA can be modified so that the Cas9 guide RNA can target a Cas9 protein to any desired sequence of any desired target nucleic acid, with the exception that the protospacer adjacent motif (PAM) sequence can be taken into account. Thus, for example, a Cas9 guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some cases, a Cas9 guide RNA includes two separate nucleic acid molecules: an “activator” and a “targeter” and is referred to herein as a “dual Cas9 guide RNA”, a “double-molecule Cas9 guide RNA”, or a “two-molecule Cas9 guide RNA” a “dual guide RNA”, or a “dgRNA.” In some cases, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

A Cas9 guide RNA comprises a crRNA-like (“CRISPR RNA”/“targeter”/“crRNA”/“crRNA repeat”) molecule and a corresponding tracrRNA-like (“trans-acting CRISPR RNA”/“activator”/“tracrRNA”) molecule. A crRNA-like molecule (targeter) comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator/tracrRNA) comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the Cas9 guide RNA. As such, each targeter molecule can be said to have a corresponding activator molecule (which has a region that hybridizes with the targeter). The targeter molecule additionally provides the targeting segment. Thus, a targeter and an activator molecule (as a corresponding pair) hybridize to form a Cas9 guide RNA. The exact sequence of a given crRNA or tracrRNA molecule is characteristic of the species in which the RNA molecules are found. A subject dual Cas9 guide RNA can include any corresponding activator and targeter pair. In some cases, a guide RNA is a multicomponent guide RNA. In some cases, a system of 2 or more or 3 or more polynucleotides is used to complex with a Cas enzyme (e.g., three split-nexus Cas associated polynucleotides). Non-limiting examples of three split-nexus Cas associated polypeptides can be found in U.S. Patent Application Publication No. 20170335347, which is hereby incorporated by reference in its entirety.

The term “activator” or “activator RNA” is used herein to mean a tracrRNA-like molecule (tracrRNA: “trans-activating CRISPR RNA”) of a Cas9 dual guide RNA (and therefore of a Cas9 single guide RNA when the “activator” and the “targeter” are linked together by, e.g., intervening nucleotides). Thus, for example, a Cas9 guide RNA (dgRNA or sgRNA) comprises an activator fsequence (e.g., a tracrRNA sequence). A tracr molecule (a tracrRNA) is a naturally existing molecule that hybridizes with a CRISPR RNA molecule (a crRNA) to form a Cas9 dual guide RNA. The term “activator” is used herein to encompass naturally existing tracrRNAs, but also to encompass tracrRNAs with modifications (e.g., truncations, sequence variations, base modifications, backbone modifications, linkage modifications, etc.) where the activator retains at least one function of a tracrRNA (e.g., contributes to the dsRNA duplex to which Cas9 protein binds). In some cases the activator provides one or more stem loops that can interact with Cas9 protein. An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a Cas9 dual guide RNA (and therefore of a Cas9 single guide RNA when the “activator” and the “targeter” are linked together, e.g., by intervening nucleotides). Thus, for example, a Cas9 guide RNA (dgRNA or sgRNA) comprises a targeting segment (which includes nucleotides that hybridize with (are complementary to) a target nucleic acid, and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, the duplex-forming segment of a targeter (described in more detail below), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

A Cas9 guide RNA can also be said to include 3 parts: (i) a targeting sequence (a nucleotide sequence that hybridizes with a sequence of the target nucleic acid); (ii) an activator sequence (as described above)(in some cases, referred to as a tracr sequence); and (iii) a sequence that hybridizes to at least a portion of the activator sequence to form a double stranded duplex. A targeter has (i) and (iii); while an activator has (ii).

A Cas9 guide RNA (e.g. a dual guide RNA or a single guide RNA) can be comprised of any corresponding activator and targeter pair. In some cases, the duplex forming segments can be swapped between the activator and the targeter. In other words, in some cases, the targeter includes a sequence of nucleotides from a duplex forming segment of a tracrRNA (which sequence would normally be part of an activator) while the activator includes a sequence of nucleotides from a duplex forming segment of a crRNA (which sequence would normally be part of a targeter).

As noted above, a targeter comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator) comprises a stretch of nucleotides (a duplex-forming segment) that forms the other half of the dsRNA duplex of the protein-binding segment of the Cas9 guide RNA. In other words, a stretch of nucleotides of the targeter is complementary to and hybridizes with a stretch of nucleotides of the activator to form the dsRNA duplex of the protein-binding segment of a Cas9 guide RNA. As such, each targeter can be said to have a corresponding activator (which has a region that hybridizes with the targeter). The targeter molecule additionally provides the targeting segment. Thus, a targeter and an activator (as a corresponding pair) hybridize to form a Cas9 guide RNA. The particular sequence of a given naturally existing crRNA or tracrRNA molecule is characteristic of the species in which the RNA molecules are found. Examples of suitable activator and targeter are well known in the art. Targeting segment of a Cas9 guide RNA

The first segment of a subject guide nucleic acid includes a guide sequence (i.e., a targeting sequence)(a nucleotide sequence that is complementary to a sequence (a target site) in a target nucleic acid). In other words, the targeting segment of a subject guide nucleic acid can interact with a target nucleic acid (e.g., double stranded DNA (dsDNA)) in a sequence-specific manner via hybridization (i.e., base pairing). As such, the nucleotide sequence of the targeting segment may vary (depending on the target) and can determine the location within the target nucleic acid that the Cas9 guide RNA and the target nucleic acid will interact. The targeting segment of a Cas9 guide RNA can be modified (e.g., by genetic engineering)/designed to hybridize to any desired sequence (target site) within a target nucleic acid (e.g., a eukaryotic target nucleic acid such as genomic DNA).

The targeting segment can have a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

The nucleotide sequence (the targeting sequence) of the targeting segment that is complementary to a nucleotide sequence (target site) of the target nucleic acid can have a length of 10 nt or more. For example, the targeting sequence of the targeting segment that is complementary to a target site of the target nucleic acid can have a length of 12 nt or more, 15 nt or more, 18 nt or more, 19 nt or more, or 20 nt or more. In some cases, the nucleotide sequence (the targeting sequence) of the targeting segment that is complementary to a nucleotide sequence (target site) of the target nucleic acid has a length of 12 nt or more. In some cases, the nucleotide sequence (the targeting sequence) of the targeting segment that is complementary to a nucleotide sequence (target site) of the target nucleic acid has a length of 18 nt or more.

For example, the targeting sequence of the targeting segment that is complementary to a target sequence of the target nucleic acid can have a length of from 10 to 100 nucleotides (nt) (e.g., from 10 to 90 nt, from 10 to 75 nt, from 10 to 60 nt, from 10 to 50 nt, from 10 to 35 nt, from 10 to 30 nt, from 10 to 25 nt, from 10 to 22 nt, from 10 to 20 nt, from 12 to 100 nt, from 12 to 90 nt, from 12 to 75 nt, from 12 to 60 nt, from 12 to 50 nt, from 12 to 35 nt, from 12 to 30 nt, from 12 to 25 nt, from 12 to 22 nt, from 12 to 20 nt, from 15 to 100 nt, from 15 to 90 nt, from 15 to 75 nt, from 15 to 60 nt, from 15 to 50 nt, from 15 to 35 nt, from 15 to 30 nt, from 15 to 25 nt, from 15 to 22 nt, from 15 to 20 nt, from 17 to 100 nt, from 17 to 90 nt, from 17 to 75 nt, from 17 to 60 nt, from 17 to 50 nt, from 17 to 35 nt, from 17 to 30 nt, from 17 to 25 nt, from 17 to 22 nt, from 17 to 20 nt, from 18 to 100 nt, from 18 to 90 nt, from 18 to 75 nt, from 18 to 60 nt, from 18 to 50 nt, from 18 to 35 nt, from 18 to 30 nt, from 18 to 25 nt, from 18 to 22 nt, or from 18 to 20 nt). In some cases, the targeting sequence of the targeting segment that is complementary to a target sequence of the target nucleic acid has a length of from 15 nt to 30 nt. In some cases, the targeting sequence of the targeting segment that is complementary to a target sequence of the target nucleic acid has a length of from 15 nt to 25 nt. In some cases, the targeting sequence of the targeting segment that is complementary to a target sequence of the target nucleic acid has a length of from 18 nt to 30 nt. In some cases, the targeting sequence of the targeting segment that is complementary to a target sequence of the target nucleic acid has a length of from 18 nt to 25 nt. In some cases, the targeting sequence of the targeting segment that is complementary to a target sequence of the target nucleic acid has a length of from 18 nt to 22 nt. In some cases, the targeting sequence of the targeting segment that is complementary to a target site of the target nucleic acid is 20 nucleotides in length. In some cases, the targeting sequence of the targeting segment that is complementary to a target site of the target nucleic acid is 19 nucleotides in length.

The percent complementarity between the targeting sequence (guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some cases, the percent complementarity between the targeting sequence of the targeting segment and the target site of the target nucleic acid is 100% over the seven contiguous 5′-most nucleotides of the target site of the target nucleic acid. In some cases, the percent complementarity between the targeting sequence of the targeting segment and the target site of the target nucleic acid is 60% or more over about 20 contiguous nucleotides. In some cases, the percent complementarity between the targeting sequence of the targeting segment and the target site of the target nucleic acid is 100% over the fourteen contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the seven contiguous 5′-most nucleotides of the target site of the target nucleic acid and as low as 0% or more over the remainder. In such a case, the targeting sequence can be considered to be 20 nucleotides in length.

In some cases, the percent complementarity between the targeting sequence of the targeting segment and the target site of the target nucleic acid is 100% over the 7 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 8 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 9 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 10 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 17 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 18 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 60% or more (e.g., e.g., 65% or more, 70% or more, 75% or more, 80% or more, 85% or more, 90% or more, 95% or more, 97% or more, 98% or more, 99% or more, or 100%) over about 20 contiguous nucleotides.

In some cases, the percent complementarity between the targeting sequence of the targeting segment and the target site of the target nucleic acid is 100% over the 7 contiguous 5′-most nucleotides of the target site of the target nucleic acid and as low as 0% or more over the remainder. In such a case, the targeting sequence can be considered to be 7 nucleotides in length. In some cases, the percent complementarity between the targeting sequence of the targeting segment and the target site of the target nucleic acid is 100% over the 8 contiguous 5′-most nucleotides of the target site of the target nucleic acid and as low as 0% or more over the remainder. In such a case, the targeting sequence can be considered to be 8 nucleotides in length. In some cases, the percent complementarity between the targeting sequence of the targeting segment and the target site of the target nucleic acid is 100% over the 9 contiguous 5′-most nucleotides of the target site of the target nucleic acid and as low as 0% or more over the remainder. In such a case, the targeting sequence can be considered to be 9 nucleotides in length. In some cases, the percent complementarity between the targeting sequence of the targeting segment and the target site of the target nucleic acid is 100% over the 10 contiguous 5′-most nucleotides of the target site of the target nucleic acid and as low as 0% or more over the remainder. In such a case, the targeting sequence can be considered to be 10 nucleotides in length. In some cases, the percent complementarity between the targeting sequence of the targeting segment and the target site of the target nucleic acid is 100% over the 11 contiguous 5′-most nucleotides of the target site of the target nucleic acid and as low as 0% or more over the remainder. In such a case, the targeting sequence can be considered to be 11 nucleotides in length. In some cases, the percent complementarity between the targeting sequence of the targeting segment and the target site of the target nucleic acid is 100% over the 12 contiguous 5′-most nucleotides of the target site of the target nucleic acid and as low as 0% or more over the remainder. In such a case, the targeting sequence can be considered to be 12 nucleotides in length. In some cases, the percent complementarity between the targeting sequence of the targeting segment and the target site of the target nucleic acid is 100% over the 13 contiguous 5′-most nucleotides of the target site of the target nucleic acid and as low as 0% or more over the remainder. In such a case, the targeting sequence can be considered to be 13 nucleotides in length. In some cases, the percent complementarity between the targeting sequence of the targeting segment and the target site of the target nucleic acid is 100% over the 14 contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the 17 contiguous 5′-most nucleotides of the target site of the target nucleic acid and as low as 0% or more over the remainder. In such a case, the targeting sequence can be considered to be 17 nucleotides in length. In some cases, the percent complementarity between the targeting sequence of the targeting segment and the target site of the target nucleic acid is 100% over the 18 contiguous 5′-most nucleotides of the target site of the target nucleic acid and as low as 0% or more over the remainder. In such a case, the targeting sequence can be considered to be 18 nucleotides in length.

Examples of various Cas9 proteins and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

### Guide RNAs Corresponding to Type V and Type VI CRISPR/Cas Endonucleases (e.g., Cpf1 Guide RNA)

A guide RNA that binds to a type V or type VI CRISPR/Cas protein (e.g., Cpf1, C2c1, C2c2, C2c3), and targets the complex to a specific location within a target nucleic acid is referred to herein generally as a “type V or type VI CRISPR/Cas guide RNA”. An example of a more specific term is a “Cpf1 guide RNA.”

A type V or type VI CRISPR/Cas guide RNA (e.g., cpf1 guide RNA) can have a total length of from 30 nucleotides (nt) to 200 nt, e.g., from 30 nt to 180 nt, from 30 nt to 160 nt, from 30 nt to 150 nt, from 30 nt to 125 nt, from 30 nt to 100 nt, from 30 nt to 90 nt, from 30 nt to 80 nt, from 30 nt to 70 nt, from 30 nt to 60 nt, from 30 nt to 50 nt, from 50 nt to 200 nt, from 50 nt to 180 nt, from 50 nt to 160 nt, from 50 nt to 150 nt, from 50 nt to 125 nt, from 50 nt to 100 nt, from 50 nt to 90 nt, from 50 nt to 80 nt, from 50 nt to 70 nt, from 50 nt to 60 nt, from 70 nt to 200 nt, from 70 nt to 180 nt, from 70 nt to 160 nt, from 70 nt to 150 nt, from 70 nt to 125 nt, from 70 nt to 100 nt, from 70 nt to 90 nt, or from 70 nt to 80 nt). In some cases, a type V or type VI CRISPR/Cas guide RNA (e.g., cpf1 guide RNA) has a total length of at least 30 nt (e.g., at least 40 nt, at least 50 nt, at least 60 nt, at least 70 nt, at least 80 nt, at least 90 nt, at least 100 nt, or at least 120 nt).

In some cases, a Cpf1 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt.

Like a Cas9 guide RNA, a type V or type VI CRISPR/Cas guide RNA (e.g., cpf1 guide RNA) can include a target nucleic acid-binding segment and a duplex-forming region (e.g., in some cases formed from two duplex-forming segments, i.e., two stretches of nucleotides that hybridize to one another to form a duplex).

The target nucleic acid-binding segment of a type V or type VI CRISPR/Cas guide RNA (e.g., cpf1 guide RNA) can have a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some cases, the target nucleic acid-binding segment has a length of 23 nt. In some cases, the target nucleic acid-binding segment has a length of 24 nt. In some cases, the target nucleic acid-binding segment has a length of 25 nt.

The guide sequence of a type V or type VI CRISPR/Cas guide RNA (e.g., cpf1 guide RNA) can have a length of from 15 nt to 30 nt (e.g., 15 to 25 nt, 15 to 24 nt, 15 to 23 nt, 15 to 22 nt, 15 to 21 nt, 15 to 20 nt, 15 to 19 nt, 15 to 18 nt, 17 to 30 nt, 17 to 25 nt, 17 to 24 nt, 17 to 23 nt, 17 to 22 nt, 17 to 21 nt, 17 to 20 nt, 17 to 19 nt, 17 to 18 nt, 18 to 30 nt, 18 to 25 nt, 18 to 24 nt, 18 to 23 nt, 18 to 22 nt, 18 to 21 nt, 18 to 20 nt, 18 to 19 nt, 19 to 30 nt, 19 to 25 nt, 19 to 24 nt, 19 to 23 nt, 19 to 22 nt, 19 to 21 nt, 19 to 20 nt, 20 to 30 nt, 20 to 25 nt, 20 to 24 nt, 20 to 23 nt, 20 to 22 nt, 20 to 21 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt. In some cases, the guide sequence has a length of 24 nt.

The guide sequence of a type V or type VI CRISPR/Cas guide RNA (e.g., cpf1 guide RNA) can have 100% complementarity with a corresponding length of target nucleic acid sequence. The guide sequence can have less than 100% complementarity with a corresponding length of target nucleic acid sequence. For example, the guide sequence of a type V or type VI CRISPR/Cas guide RNA (e.g., cpf1 guide RNA) can have 1, 2, 3, 4, or 5 nucleotides that are not complementary to the target nucleic acid sequence. For example, in some cases, where a guide sequence has a length of 25 nucleotides, and the target nucleic acid sequence has a length of 25 nucleotides, in some cases, the target nucleic acid-binding segment has 100% complementarity to the target nucleic acid sequence. As another example, in some cases, where a guide sequence has a length of 25 nucleotides, and the target nucleic acid sequence has a length of 25 nucleotides, in some cases, the target nucleic acid-binding segment has 1 non-complementary nucleotide and 24 complementary nucleotides with the target nucleic acid sequence. As another example, in some cases, where a guide sequence has a length of 25 nucleotides, and the target nucleic acid sequence has a length of 25 nucleotides, in some cases, the target nucleic acid-binding segment has 2 non-complementary nucleotides and 23 complementary nucleotides with the target nucleic acid sequence.

The duplex-forming segment of a type V or type VI CRISPR/Cas guide RNA (e.g., cpf1 guide RNA) (e.g., of a targeter RNA or an activator RNA) can have a length of from 15 nt to 25 nt (e.g., 15 nt, 16 nt, 17 nt, 18 nt, 19 nt, 20 nt, 21 nt, 22 nt, 23 nt, 24 nt, or 25 nt).

The RNA duplex of a type V or type VI CRISPR/Cas guide RNA (e.g., cpf1 guide RNA) can have a length of from 5 base pairs (bp) to 40 bp (e.g., from 5 to 35 bp, 5 to 30 bp, 5 to 25 bp, 5 to 20 bp, 5 to 15 bp, 5-12 bp, 5-10 bp, 5-8 bp, 6 to 40 bp, 6 to 35 bp, 6 to 30 bp, 6 to 25 bp, 6 to 20 bp, 6 to 15 bp, 6 to 12 bp, 6 to 10 bp, 6 to 8 bp, 7 to 40 bp, 7 to 35 bp, 7 to 30 bp, 7 to 25 bp, 7 to 20 bp, 7 to 15 bp, 7 to 12 bp, 7 to 10 bp, 8 to 40 bp, 8 to 35 bp, 8 to 30 bp, 8 to 25 bp, 8 to 20 bp, 8 to 15 bp, 8 to 12 bp, 8 to 10 bp, 9 to 40 bp, 9 to 35 bp, 9 to 30 bp, 9 to 25 bp, 9 to 20 bp, 9 to 15 bp, 9 to 12 bp, 9 to 10 bp, 10 to 40 bp, 10 to 35 bp, 10 to 30 bp, 10 to 25 bp, 10 to 20 bp, 10 to 15 bp, or 10 to 12 bp).

As an example, a duplex-forming segment of a Cpf1 guide RNA can comprise a nucleotide sequence selected from (5′ to 3′): AAUUUCUACUGUUGUAGAU (SEQ ID NO: 829), AAUUUCUGCUGUUGCAGAU (SEQ ID NO: 830), AAUUUCCACUGUUGUGGAU (SEQ ID NO: 831), AAUUCCUACUGUUGUAGGU (SEQ ID NO: 832), AAUUUCUACUAUUGUAGAU (SEQ ID NO: 833), AAUUUCUACUGCUGUAGAU (SEQ ID NO: 834), AAUUUCUACUUUGUAGAU (SEQ ID NO: 835), and AAUUUCUACUUGUAGAU (SEQ ID NO: 836). The guide sequence can then follow (5′ to 3′) the duplex forming segment.

A non-limiting example of an activator RNA (e.g. tracrRNA) of a C2c1 guide RNA (dual guide or single guide) is an RNA that includes the nucleotide sequence GAAUUUUUCAACGGGUGUGCCAAUGGCCACUUUCCAGGUGGCAAAGCCCGUUGA GCUUCUCAAAAAG (SEQ ID NO: 837). In some cases, a C2c1 guide RNA (dual guide or single guide) is an RNA that includes the nucleotide sequence In some cases, a C2c1 guide RNA (dual guide or single guide) is an RNA that includes the nucleotide sequence GUCUAGAGGACAGAAUUUUUCAACGGGUGUGCCAAUGGCCACUUUCCAGGUGGC AAAGCCCGUUGAGCUUCUCAAAAAG (SEQ ID NO: 838). In some cases, a C2c1 guide RNA (dual guide or single guide) is an RNA that includes the nucleotide sequence UCUAGAGGACAGAAUUUUUCAACGGGUGUGCCAAUGGCCACUUUCCAGGUGGCA AAGCCCGUUGAGCUUCUCAAAAAG (SEQ ID NO: 839). A non-limiting example of an activator RNA (e.g. tracrRNA) of a C2c1 guide RNA (dual guide or single guide) is an RNA that includes the nucleotide sequence ACUUUCCAGGCAAAGCCCGUUGAGCUUCUCAAAAAG (SEQ ID NO: 840). In some cases, a duplex forming segment of a C2c1 guide RNA (dual guide or single guide) of an activator RNA (e.g. tracrRNA) includes the nucleotide sequence AGCUUCUCA (SEQ ID NO: 841) or the nucleotide sequence GCUUCUCA (SEQ ID NO: 842) (the duplex forming segment from a naturally existing tracrRNA.

A non-limiting example of a targeter RNA (e.g. crRNA) of a C2c1 guide RNA (dual guide or single guide) is an RNA with the nucleotide sequence CUGAGAAGUGGCACNNNNNNNNNNNNNNNNNNNN (SEQ ID NO: 843), where the Ns represent the guide sequence, which will vary depending on the target sequence, and although 20 Ns are depicted a range of different lengths are acceptable. In some cases, a duplex forming segment of a C2c1 guide RNA (dual guide or single guide) of a targeter RNA (e.g. crRNA) includes the nucleotide sequence CUGAGAAGUGGCAC (SEQ ID NO: 844) or includes the nucleotide sequence CUGAGAAGU (SEQ ID NO: 845) or includes the nucleotide sequence UGAGAAGUGGCAC (SEQ ID NO: 846) or includes the nucleotide sequence UGAGAAGU (SEQ ID NO: 847).

Examples and guidance related to type V or type VI CRISPR/Cas endonucleases and guide RNAs (as well as information regarding requirements related to protospacer adjacent motif (PAM) sequences present in targeted nucleic acids) can be found in the art, for example, see Zetsche et al., Cell. 2015 Oct. 22; 163(3):759-71; Makarova et al., Nat Rev Microbiol. 2015 November; 13(11):722-36; and Shmakov et al., Mol Cell. 2015 Nov. 5; 60(3):385-97.

### Nucleic Acid Modifications

In some cases, a modified cargo RNA of a system of the present disclosure comprises one or more modifications, e.g., a base modification, a backbone modification, a sugar modification, etc., to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′Omethyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

In some cases, 2% or more of the nucleotides of a modified cargo RNA (e.g., a guide RNA, etc.) are modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject nucleic acid are modified). In some cases, 2% or more of the nucleotides of a subject guide RNA are modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a subject guide RNA are modified). In some cases, 2% or more of the nucleotides of a guide RNA are modified (e.g., 3% or more, 5% or more, 7.5% or more, 10% or more, 15% or more, 20% or more, 25% or more, 30% or more, 35% or more, 40% or more, 45% or more, 50% or more, 55% or more, 60% or more, 65% or more, 75% or more, 80% or more, 85% or more, 90% or more, 95% or more, or 100% of the nucleotides of a guide RNA are modified).

In some cases, the number of nucleotides of a modified cargo RNA (e.g., a guide RNA, etc.) that are modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a modified cargo RNA (e.g., guide RNA) that are modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%). In some cases, the number of nucleotides of a modified cargo RNA (e.g., guide RNA) that are modified is in a range of from 3% to 100% (e.g., 3% to 100%, 3% to 95%, 3% to 90%, 3% to 85%, 3% to 80%, 3% to 75%, 3% to 70%, 3% to 65%, 3% to 60%, 3% to 55%, 3% to 50%, 3% to 45%, 3% to 40%, 5% to 100%, 5% to 95%, 5% to 90%, 5% to 85%, 5% to 80%, 5% to 75%, 5% to 70%, 5% to 65%, 5% to 60%, 5% to 55%, 5% to 50%, 5% to 45%, 5% to 40%, 10% to 100%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, or 10% to 40%).

In some cases, one or more of the nucleotides of a modified cargo RNA (e.g., guide RNA) are modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a modified cargo RNA are modified). In some cases, one or more of the nucleotides of a modified cargo RNA are modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a modified cargo RNA are modified). In some cases, one or more of the nucleotides of a guide RNA are modified (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or all of the nucleotides of a guide RNA are modified).

In some cases, 99% or less of the nucleotides of a modified cargo RNA (e.g., a guide RNA, etc.) are modified (e.g., 99% or less, 95% or less, 90% or less, 85% or less, 80% or less, 75% or less, 70% or less, 65% or less, 60% or less, 55% or less, 50% or less, or 45% or less of the nucleotides of a modified cargo RNA are modified). In some cases, 99% or less of the nucleotides of a modified cargo RNA are modified (e.g., e.g., 99% or less, 95% or less, 90% or less, 85% or less, 80% or less, 75% or less, 70% or less, 65% or less, 60% or less, 55% or less, 50% or less, or 45% or less of the nucleotides of a modified cargo RNA are modified). In some cases, 99% or less of the nucleotides of a guide RNA are modified (e.g., 99% or less, 95% or less, 90% or less, 85% or less, 80% or less, 75% or less, 70% or less, 65% or less, 60% or less, 55% or less, 50% or less, or 45% or less of the nucleotides of a guide RNA are modified).

In some cases, the number of nucleotides of a modified cargo RNA (e.g., a guide RNA, etc.) that are modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a modified cargo RNA that are modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10). In some cases, the number of nucleotides of a modified cargo RNA that are modified is in a range of from 1 to 30 (e.g., 1 to 25, 1 to 20, 1 to 18, 1 to 15, 1 to 10, 2 to 25, 2 to 20, 2 to 18, 2 to 15, 2 to 10, 3 to 25, 3 to 20, 3 to 18, 3 to 15, or 3 to 10).

In some cases, 20 or fewer of the nucleotides of a modified cargo RNA (e.g., a guide RNA, etc.) are modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a modified cargo RNA are modified). In some cases, 20 or fewer of the nucleotides of a modified cargo RNA are modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a modified cargo RNA are modified). In some cases, 20 or fewer of the nucleotides of a modified cargo RNA are modified (e.g., 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or one, of the nucleotides of a guide RNA are modified).

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases Tm of RNA:RNA duplexes but results in only small changes in RNA:DNA stability. It is stable with respect to attack by single-stranded ribonucleases and is typically 5 to 10-fold less susceptible to DNases than DNA. It is commonly used in antisense oligos as a means to increase stability and binding affinity to the target message.

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siRNAs to improve stability in serum or other biological fluids.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an oligo at any position except the 3′-end. Applications have been described ranging from antisense oligos to hybridization probes to SNP detection and allele specific PCR. Due to the large increase in Tm conferred by LNAs, they also can cause an increase in primer dimer formation as well as self-hairpin formation. In some cases, the number of LNAs incorporated into a single oligo is 10 bases or less.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some cases, a modified cargo RNA (e.g., a guide RNA, etc.) has one or more nucleotides that are 2′-O-Methyl modified nucleotides. In some case, a modified cargo RNA acid (e.g., a guide RNA, etc.) has one or more 2′ Fluoro modified nucleotides. In some cases, a modified cargo RNA (e.g., a guide RNA, etc.) has one or more LNA bases. In some cases, a modified cargo RNA (e.g., a guide RNA, etc.) has one or more nucleotides that are linked by a phosphorothioate bond (i.e., the modified cargo RNA has one or more phosphorothioate linkages). In some cases, a modified cargo RNA (e.g., a guide RNA, etc.) has a 5′ cap (e.g., a 7-methylguanylate cap (m7G)).

In some cases, a modified cargo RNA has a combination of modified nucleotides. For example, a nucleic acid can have a 5′ cap (e.g., a 7-methylguanylate cap (m7G)) in addition to having one or more nucleotides with other modifications (e.g., a 2′-O-Methyl nucleotide and/or a 2′ Fluoro modified nucleotide and/or a LNA base and/or a phosphorothioate linkage).

Modified Backbones and Modified Internucleoside Linkages

In some cases, a modified cargo RNA comprises a modified backbone and/or modified internucleoside linkages. Examples of suitable nucleic acids containing modifications include nucleic acids containing modified backbones or non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a modified cargo RNA comprises one or more phosphorothioate and/or heteroatom internucleoside linkages, in particular —CH₂—NH—O—CH₂—, —CH₂—N(CH₃)—O—CH₂-(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are morpholino backbone structures as described in, e.g., U.S. Pat. No. 5,034,506. For example, in some cases, a modified cargo RNA comprises a 6-membered morpholino ring in place of a ribose ring. In some of these cases, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Mimetics

A modified cargo RNA can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630). LNAs and preparation thereof are also described in WO 98/39352 and WO 99/14226, as well as U.S. applications 20120165514, 20100216983, 20090041809, 20060117410, 20040014959, 20020094555, and 20020086998.

Modified Sugar Moieties

A modified cargo RNA can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethyl-amino-ethoxy-ethyl or 2′-DMAEOE), i.e., 2′-O—CH₂—O—CH₂-N(CH₃)2.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

Base Modifications and Substitutions

A modified cargo RNA may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

### RNA-Guided CRISPR/Cas Effector Proteins

As noted above, a system of the present disclosure comprises: a) a modified RBP comprising: i) an RBP; and ii) one or more endosomolytic peptides covalently linked, directly or via a linker, to the RBP; and b) a modified cargo RNA complexed to the RBP, wherein the modified cargo RNA comprises a cargo RNA modified to include one or more RBP binding sites that are bound by the RBP present in the modified RBP. As noted above, in some cases, the cargo RNA is a guide RNA comprising: i) a targeting segment comprising a nucleotide sequence that hybridizes to a nucleotide sequence in a target nucleic acid; and ii) an activation segment comprising a nucleotide sequence that binds to and activates an RNA-guided effector polypeptide. As noted above, in some cases, the system comprises an RNA-guided effector polypeptide. The RNA-guided effector polypeptide can form a complex with the guide RNA portion of the modified cargo RNA. In some cases, the RNA-guided effector polypeptide includes a targeting moiety, e.g., a moiety that provides for selective binding to a particular cell type. In some cases, the targeting moiety is a chemical compound, a peptide, or an antibody.

Suitable RNA-guided CRISPR/Cas effector polypeptides include, but are not limited to, a class 2 CRISPR/Cas effector polypeptide (e.g., a type II CRISPR/Cas effector polypeptide, a Cas9 protein, a type V or type VI CRISPR/Cas effector polypeptide, a Cpf1 protein, a C2c1 protein, a C2c3 protein, a C2c2 protein, a Cas12 enzyme, or a Cas13 enzyme).

In certain cases, the RNA-guided CRISPR/Cas effector polypeptide is a CRISPR/Cas endonuclease (e.g., class 2 CRISPR/Cas endonucleases such as a type II, type V, or type VI CRISPR/Cas endonucleases). In some cases, a suitable RNA-guided CRISPR/Cas effector polypeptide is a class 2 CRISPR/Cas endonuclease. In some cases, a suitable RNA-guided CRISPR/Cas effector polypeptide is a class 2 type II CRISPR/Cas endonuclease (e.g., a Cas9 protein). In some cases, a suitable RNA-guided CRISPR/Cas effector polypeptide is a class 2 type V CRISPR/Cas endonuclease (e.g., a Cpf1 protein, a C2c1 protein, or a C2c3 protein). In some cases, a suitable RNA-guided CRISPR/Cas effector polypeptide is a class 2 type VI CRISPR/Cas endonuclease (e.g., a C2c2 protein; also referred to as a “Cas13a” protein). Also suitable for use is a CasX protein. Also suitable for use is a CasY protein.

In some cases, the RNA-guided CRISPR/Cas effector polypeptide is a Type II CRISPR/Cas endonuclease. In some cases, the RNA-guided CRISPR/Cas effector polypeptide is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 depicted in FIG. 5A. In some cases, a Cas9 polypeptide comprises the amino acid sequence depicted in one of FIG. 5A-5F.

In some cases, the Cas9 polypeptide used in a system or method of the present disclosure is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence depicted in FIG. 6.

In some cases, the Cas9 polypeptide used in a system or method of the present disclosure is a Campylobacter jejuni Cas9 (CjCas9) polypeptide. CjCas9 recognizes the 5′-NNNVRYM-3′ as the protospacer-adjacent motif (PAM). In some cases, a Cas9 polypeptide suitable for use in a system or method of the present disclosure comprises an amino acid sequence having at least 50%, at least 60%, at least 70%, at least 80%, at least 90%, at least 95%, at least 98%, at least 99%, or more than 99%, amino acid sequence identity to the CjCas9 amino acid sequence set forth in SEQ ID NO:55.

In some cases, a suitable Cas9 polypeptide is a high-fidelity (HF) Cas9 polypeptide. Kleinstiver et al. (2016) Nature 529:490. For example, amino acids N₄₉₇, R661, Q695, and Q926 of the amino acid sequence depicted in FIG. 5A are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 5A, where amino acids N₄₉₇, R661, Q695, and Q926 are substituted, e.g., with alanine.

In some cases, a suitable Cas9 polypeptide exhibits altered PAM specificity. See, e.g., Kleinstiver et al. (2015) Nature 523:481.

In some cases, the RNA-guided CRISPR/Cas effector polypeptide is a type V CRISPR/Cas endonuclease. In some cases, a type V CRISPR/Cas endonuclease is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 7A, FIG. 7B, or FIG. 7C.

In some cases, the RNA-guided CRISPR/Cas effector polypeptide is a CasX or a CasY polypeptide. CasX and CasY polypeptides are described in Burstein et al. (2017) Nature 542:237.

In some cases, an RNA-guided CRISPR/Cas effector polypeptide is a fusion protein that comprises the RNA-guided CRISPR/Cas effector polypeptide fused to a heterologous polypeptide (also referred to as a “fusion partner”). In some cases, the fusion partner provides for subcellular localization, i.e., the fusion partner is a subcellular localization sequence (e.g., one or more nuclear localization signals (NLSs) for targeting to the nucleus, two or more NLSs, three or more NLSs, etc.).

In some cases, a suitable Cas9 polypeptide is a high-fidelity (HF) Cas9 polypeptide. Kleinstiver et al. (2016) Nature 529:490. For example, amino acids N₄₉₇, R661, Q695, and Q926 of the amino acid sequence depicted in FIG. 5A are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 5A, where amino acids N₄₉₇, R661, Q695, and Q926 are substituted, e.g., with alanine.

In some cases, a suitable Cas9 polypeptide exhibits altered PAM specificity. See, e.g., Kleinstiver et al. (2015) Nature 523:481.

In some cases, the genome-editing endonuclease is a type V CRISPR/Cas endonuclease. In some cases, a type V CRISPR/Cas endonuclease is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 7A. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 7B. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 7C.

A nucleic acid that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a nucleotide sequence present in a target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.” In some cases, the guide RNA is a multi-component guide RNA. In some cases, the multi-component guide RNA comprises 2 or more, or 3 or more polynucleotides (e.g. split-nexus polynucleotides). Non-limiting examples of three split-nexus polynucleotides can be found in U.S. Patent Application Publication No. 20170335347, which is hereby incorporated by reference in its entirety.

In some cases, a system of the present disclosure comprises an RNA-guided CRISPR/Cas effector polypeptide, where the RNA-guided CRISPR/Cas effector polypeptide may be complexed with the guide RNA portion of the modified cargo RNA. In some cases, e.g., where a target nucleic acid comprises a deleterious mutation in a defective allele (e.g., a deleterious mutation in a retinal cell target nucleic acid), the RNA-guided endonuclease/guide RNA complex, together with a donor nucleic acid comprising a nucleotide sequence that corrects the deleterious mutation (e.g., a donor nucleic acid comprising a nucleotide sequence that encodes a functional copy of the protein encoded by the defective allele), can be used to correct the deleterious mutation, e.g., via homology-directed repair (HDR).

In some cases, a system of the present disclosure comprises: i) an RNA-guided CRISPR/Cas effector polypeptide; and ii) one modified guide RNA (a guide RNA modified to include binding sites for the RBP). In some cases, the guide RNA is a single-molecule (or “single guide”) guide RNA (an “sgRNA”). In some cases, the guide RNA is a dual-molecule (or “dual-guide”) guide RNA (“dgRNA”). In some cases, the guide RNA is a multi-component guide RNA. In some cases, the multi-component guide RNA is one or more split-nexus polynucleotides.

In some cases, a system of the present disclosure comprises: i) RNA-guided CRISPR/Cas effector polypeptide; and ii) 2 separate sgRNAs, where the 2 separate sgRNAs provide for deletion of a target nucleic acid via non-homologous end joining (NHEJ). In some cases, the guide RNAs are sgRNAs. In some cases, the guide RNAs are dgRNAs.

### Class 2 CRISPR/Cas Endonucleases

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. In class 2 CRISPR systems, the functions of the effector complex (e.g., the cleavage of target DNA) are carried out by a single endonuclease (e.g., see Zetsche et al., Cell. 2015 Oct. 22; 163(3):759-71; Makarova et al., Nat Rev Microbiol. 2015 November; 13(11):722-36; Shmakov et al., Mol Cell. 2015 Nov. 5; 60(3):385-97); and Shmakov et al. (2017) Nature Reviews Microbiology 15:169. As such, the term “class 2 CRISPR/Cas protein” is used herein to encompass the endonuclease (the target nucleic acid cleaving protein) from class 2 CRISPR systems. Thus, the term “class 2 CRISPR/Cas endonuclease” as used herein encompasses type II CRISPR/Cas proteins (e.g., Cas9); type V-A CRISPR/Cas proteins (e.g., Cpf1 (also referred to a “Cas12a”)); type V-B CRISPR/Cas proteins (e.g., C2c1 (also referred to as “Cas12b”)); type V-C CRISPR/Cas proteins (e.g., C2c3 (also referred to as “Cas12c”)); type V-U1 CRISPR/Cas proteins (e.g., C2c4); type V-U2 CRISPR/Cas proteins (e.g., C2c8); type V-U5 CRISPR/Cas proteins (e.g., C2c5); type V-U4 CRISPR/Cas proteins (e.g., C2c9); type V-U3 CRISPR/Cas proteins (e.g., C2c10); type VI-A CRISPR/Cas proteins (e.g., C2c2 (also known as “Cas13a”)); type VI-B CRISPR/Cas proteins (e.g., Cas13b (also known as C2c4)); and type VI-C CRISPR/Cas proteins (e.g., Cas13c (also known as C2c7)). To date, class 2 CRISPR/Cas proteins encompass type II, type V, and type VI CRISPR/Cas proteins, but the term is also meant to encompass any class 2 CRISPR/Cas protein suitable for binding to a corresponding guide RNA and forming an RNP complex.

### Type II CRISPR/Cas Endonucleases (e.g., Cas9)

In natural Type II CRISPR/Cas systems, Cas9 functions as an RNA-guided endonuclease that uses a dual-guide RNA having a crRNA and trans-activating crRNA (tracrRNA) for target recognition and cleavage by a mechanism involving two nuclease active sites in Cas9 that together generate double-stranded DNA breaks (DSBs), or can individually generate single-stranded DNA breaks (SSBs). The Type II CRISPR endonuclease Cas9 and engineered dual- (dgRNA) or single guide RNA (sgRNA) form a ribonucleoprotein (RNP) complex that can be targeted to a desired DNA sequence. Guided by a dual-RNA complex or a chimeric single-guide RNA, Cas9 generates site-specific DSBs or SSBs within double-stranded DNA (dsDNA) target nucleic acids, which are repaired either by non-homologous end joining (NHEJ) or homology-directed recombination (HDR).

A type II CRISPR/Cas endonuclease is a type of class 2 CRISPR/Cas endonuclease. In some cases, the type II CRISPR/Cas endonuclease is a Cas9 protein. A Cas9 protein forms a complex with a Cas9 guide RNA. The guide RNA provides target specificity to a Cas9-guide RNA complex by having a nucleotide sequence (a guide sequence) that is complementary to a sequence (the target site) of a target nucleic acid (as described elsewhere herein). The Cas9 protein of the complex provides the site-specific activity. In other words, the Cas9 protein is guided to a target site (e.g., stabilized at a target site) within a target nucleic acid sequence (e.g. a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA.

A Cas9 protein can bind and/or modify (e.g., cleave, nick, methylate, demethylate, etc.) a target nucleic acid and/or a polypeptide associated with target nucleic acid (e.g., methylation or acetylation of a histone tail)(e.g., when the Cas9 protein includes a fusion partner with an activity). In some cases, the Cas9 protein is a naturally-occurring protein (e.g., naturally occurs in bacterial and/or archaeal cells). In other cases, the Cas9 protein is not a naturally-occurring polypeptide (e.g., the Cas9 protein is a variant Cas9 protein, a chimeric protein, and the like).

Examples of suitable Cas9 proteins include, but are not limited to, those set forth in SEQ ID NOs: 5-816. Naturally occurring Cas9 proteins bind a Cas9 guide RNA, are thereby directed to a specific sequence within a target nucleic acid (a target site), and cleave the target nucleic acid (e.g., cleave dsDNA to generate a double strand break, cleave ssDNA, cleave ssRNA, etc.). A chimeric Cas9 protein is a fusion protein comprising a Cas9 polypeptide that is fused to a heterologous protein (referred to as a fusion partner), where the heterologous protein provides an activity (e.g., one that is not provided by the Cas9 protein). The fusion partner can provide an activity, e.g., enzymatic activity (e.g., nuclease activity, activity for DNA and/or RNA methylation, activity for DNA and/or RNA cleavage, activity for histone acetylation, activity for histone methylation, activity for RNA modification, activity for RNA-binding, activity for RNA splicing etc.). In some cases a portion of the Cas9 protein (e.g., the RuvC domain and/or the HNH domain) exhibits reduced nuclease activity relative to the corresponding portion of a wild type Cas9 protein (e.g., in some cases the Cas9 protein is a nickase). In some cases, the Cas9 protein is enzymatically inactive, or has reduced enzymatic activity relative to a wild-type Cas9 protein (e.g., relative to Streptococcus pyogenes Cas9).

Assays to determine whether given protein interacts with a Cas9 guide RNA can be any convenient binding assay that tests for binding between a protein and a nucleic acid. Suitable binding assays (e.g., gel shift assays) will be known to one of ordinary skill in the art (e.g., assays that include adding a Cas9 guide RNA and a protein to a target nucleic acid).

Assays to determine whether a protein has an activity (e.g., to determine if the protein has nuclease activity that cleaves a target nucleic acid and/or some heterologous activity) can be any convenient assay (e.g., any convenient nucleic acid cleavage assay that tests for nucleic acid cleavage). Suitable assays (e.g., cleavage assays) will be known to one of ordinary skill in the art and can include adding a Cas9 guide RNA and a protein to a target nucleic acid.

Many Cas9 orthologs from a wide variety of species have been identified and in some cases the proteins share only a few identical amino acids. Identified Cas9 orthologs have similar domain architecture with a central HNH endonuclease domain and a split RuvC/RNaseH domain (e.g., RuvCI, RuvCII, and RuvCIII) (e.g., see Table 1). For example, a Cas9 protein can have 3 different regions (sometimes referred to as RuvC-I, RuvC-II, and RucC-III), that are not contiguous with respect to the primary amino acid sequence of the Cas9 protein, but fold together to form a RuvC domain once the protein is produced and folds. Thus, Cas9 proteins can be said to share at least 4 key motifs with a conserved architecture. Motifs 1, 2, and 4 are RuvC like motifs while motif 3 is an HNH-motif. The motifs set forth in Table 1 may not represent the entire RuvC-like and/or HNH domains as accepted in the art, but Table 1 does present motifs that can be used to help determine whether a given protein is a Cas9 protein. In some cases, the methods and compositions of the present disclosure include a Cas9 ortholog as described herein.

TABLE 1
Table 1 lists 4 motifs that are present in Cas9 sequences
from various species. The amino acids listed in Table 1 are
from the Cas9 from S. pyogenes (SEQ ID NO: 5).

| Motif # | Motif | Amino acids (residue #s) | Highly conserved |
|---|---|---|---|
| 1 | RuvC-like I | IGLDIGTNSVGWAVI (7-21) | D10, G12, G17 |
| | | (SEQ ID NO: 1) | |
| 2 | RuvC-like II | IVIEMARE (759-766) | E762 |
| | | (SEQ ID NO: 2) | |
| 3 | HNH-motif | DVDHIVPQSFLKDDSIDNKVLTRSDK | H840, N854, N863 |
| | | N (837-863) (SEQ ID NO: 3) | |
| 4 | RuvC-like | HHAHDAYL (982-989) | H982, H983, A984, |
| | III | (SEQ ID NO: 4) | D986, A987 |

In some cases, a suitable Cas9 protein comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 as set forth in SEQ ID NOs: 1-4, respectively (e.g., see Table 1), or to the corresponding portions in any of the amino acid sequences set forth in SEQ ID NOs: 5-816.

In other words, in some cases, a suitable Cas9 polypeptide comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 of the Cas9 amino acid sequence set forth in SEQ ID NO: 5 (e.g., the sequences set forth in SEQ ID NOs: 1-4, e.g., see Table 1), or to the corresponding portions in any of the amino acid sequences set forth in SEQ ID NOs: 6-816.

In some cases, a suitable Cas9 protein comprises an amino acid sequence having 4 motifs, each of motifs 1-4 having 60% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 70% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 75% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 80% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 85% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 90% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 95% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 99% or more amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. In some cases, a suitable Cas9 protein comprises an amino acid sequence having 4 motifs, each of motifs 1-4 having 100% amino acid sequence identity to motifs 1-4 of the Cas9 amino acid sequence set forth as SEQ ID NO: 5 (the motifs are in Table 1, and are set forth as SEQ ID NOs: 1-4, respectively), or to the corresponding portions in any of the amino acid sequences set forth in SEQ ID NOs: 6-816. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a suitable Cas9 protein comprises an amino acid sequence having 60% or more, 70% or more, 75% or more, 80% or more, 85% or more, 90% or more, 95% or more, 99% or more or 100% amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816.

In some cases, a suitable Cas9 protein comprises an amino acid sequence having 60% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 70% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 75% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 80% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 85% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 90% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 95% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 99% or more amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. In some cases, a suitable Cas9 protein comprises an amino acid sequence having 100% amino acid sequence identity to amino acids 7-166 or 731-1003 of the Cas9 amino acid sequence set forth in SEQ ID NO: 5, or to the corresponding portions in any of the amino acid sequences set forth as SEQ ID NOs: 6-816. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a suitable Cas9 protein comprises an amino acid sequence having 60% or more, 70% or more, 75% or more, 80% or more, 85% or more, 90% or more, 95% or more, 99% or more or 100% amino acid sequence identity to the Cas9 amino acid sequence set forth in SEQ ID NO: 5, or to any of the amino acid sequences set forth as SEQ ID NOs: 6-816.

In some cases, a suitable Cas9 protein comprises an amino acid sequence having 60% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 70% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 75% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 80% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 85% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 90% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 95% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 99% or more amino acid sequence identity to the Cas9 amino acid sequence set forth in SEQ ID NO: 5, or to any of the amino acid sequences set forth as SEQ ID NOs: 6-816. In some cases, a suitable Cas9 protein comprises an amino acid sequence having 100% amino acid sequence identity to the Cas9 amino acid sequence set forth in SEQ ID NO: 5, or to any of the amino acid sequences set forth as SEQ ID NOs: 6-816. Any Cas9 protein as defined above can be used as a Cas9 polypeptide, as part of a chimeric Cas9 polypeptide (e.g., a Cas9 fusion protein), any of which can be used in an RNP of the present disclosure.

In some cases, a Cas9 protein comprises 4 motifs (as listed in Table 1), at least one with (or each with) amino acid sequences having 75% or more, 80% or more, 85% or more, 90% or more, 95% or more, 99% or more or 100% amino acid sequence identity to each of the 4 motifs listed in Table 1 (SEQ ID NOs:1-4), or to the corresponding portions in any of the amino acid sequences set forth as SEQ ID NOs: 6-816.

Examples of various Cas9 proteins (and Cas9 domain structure) and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; Shmakov et al., Nat Rev Microbiol. 2017 March; 15(3):169-182; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

Variant Cas9 Proteins—Nickases and dCas9

In some cases, a Cas9 protein is a variant Cas9 protein. A variant Cas9 protein has an amino acid sequence that is different by at least one amino acid (e.g., has a deletion, insertion, substitution, fusion) when compared to the amino acid sequence of a corresponding wild type Cas9 protein. In some instances, the variant Cas9 protein has an amino acid change (e.g., deletion, insertion, or substitution) that reduces the nuclease activity of the Cas9 protein. For example, in some instances, the variant Cas9 protein has 50% or less, 40% or less, 30% or less, 20% or less, 10% or less, 5% or less, or 1% or less of the nuclease activity of the corresponding wild-type Cas9 protein. In some cases, the variant Cas9 protein has no substantial nuclease activity. When a Cas9 protein is a variant Cas9 protein that has no substantial nuclease activity, it can be referred to as a nuclease defective Cas9 protein or “dCas9” for “dead” Cas9. A protein (e.g., a class 2 CRISPR/Cas protein, e.g., a Cas9 protein) that cleaves one strand but not the other of a double stranded target nucleic acid is referred to herein as a “nickase” (e.g., a “nickase Cas9”).

In some cases, a variant Cas9 protein can cleave the complementary strand (sometimes referred to in the art as the target strand) of a target nucleic acid but has reduced ability to cleave the non-complementary strand (sometimes referred to in the art as the non-target strand) of a target nucleic acid. For example, the variant Cas9 protein can have a mutation (amino acid substitution) that reduces the function of the RuvC domain. Thus, the Cas9 protein can be a nickase that cleaves the complementary strand, but does not cleave the non-complementary strand. As a non-limiting example, in some cases, a variant Cas9 protein has a mutation at an amino acid position corresponding to residue D10 (e.g., D10A, aspartate to alanine) of SEQ ID NO: 5 (or the corresponding position of any of the proteins set forth in SEQ ID NOs: 6-261 and 264-816) and can therefore cleave the complementary strand of a double stranded target nucleic acid but has reduced ability to cleave the non-complementary strand of a double stranded target nucleic acid (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21). See, e.g., SEQ ID NO: 262.

In some cases, a variant Cas9 protein can cleave the non-complementary strand of a target nucleic acid but has reduced ability to cleave the complementary strand of the target nucleic acid. For example, the variant Cas9 protein can have a mutation (amino acid substitution) that reduces the function of the HNH domain. Thus, the Cas9 protein can be a nickase that cleaves the non-complementary strand, but does not cleave the complementary strand. As a non-limiting example, in some cases, the variant Cas9 protein has a mutation at an amino acid position corresponding to residue H840 (e.g., an H840A mutation, histidine to alanine) of SEQ ID NO: 5 (or the corresponding position of any of the proteins set forth as SEQ ID NOs: 6-261 and 264-816) and can therefore cleave the non-complementary strand of the target nucleic acid but has reduced ability to cleave (e.g., does not cleave) the complementary strand of the target nucleic acid. Such a Cas9 protein has a reduced ability to cleave a target nucleic acid (e.g., a single stranded target nucleic acid) but retains the ability to bind a target nucleic acid (e.g., a single stranded target nucleic acid). See, e.g., SEQ ID NO: 263.

In some cases, a variant Cas9 protein has a reduced ability to cleave both the complementary and the non-complementary strands of a double stranded target nucleic acid. As a non-limiting example, in some cases, the variant Cas9 protein harbors mutations at amino acid positions corresponding to residues D10 and H840 (e.g., D10A and H840A) of SEQ ID NO: 5 (or the corresponding residues of any of the proteins set forth as SEQ ID NOs: 6-261 and 264-816) such that the polypeptide has a reduced ability to cleave (e.g., does not cleave) both the complementary and the non-complementary strands of a target nucleic acid. Such a Cas9 protein has a reduced ability to cleave a target nucleic acid (e.g., a single stranded or double stranded target nucleic acid) but retains the ability to bind a target nucleic acid. A Cas9 protein that cannot cleave target nucleic acid (e.g., due to one or more mutations, e.g., in the catalytic domains of the RuvC and HNH domains) is referred to as a “dead” Cas9 or simply “dCas9.” See, e.g., SEQ ID NO: 264.

Other residues can be mutated to achieve the above effects (i.e. inactivate one or the other nuclease portions). As non-limiting examples, residues D10, G12, G17, E762, H840, N₈₅₄, N₈₆₃, H982, H983, A984, D986, and/or A987 of SEQ ID NO: 5 (or the corresponding mutations of any of the proteins set forth as SEQ ID NOs: 6-816) can be altered (i.e., substituted). Also, mutations other than alanine substitutions are suitable.

In some cases, a variant Cas9 protein that has reduced catalytic activity (e.g., when a Cas9 protein has a D10, G12, G17, E762, H840, N₈₅₄, N₈₆₃, H982, H983, A984, D986, and/or a A987 mutation of SEQ ID NO: 5 or the corresponding mutations of any of the proteins set forth as SEQ ID NOs: 6-816, e.g., D10A, G12A, G17A, E762A, H840A, N₈₅₄A, N₈₆₃A, H982A, H983A, A984A, and/or D986A), the variant Cas9 protein can still bind to target nucleic acid in a site-specific manner (because it is still guided to a target nucleic acid sequence by a Cas9 guide RNA) as long as it retains the ability to interact with the Cas9 guide RNA.

In addition to the above, a variant Cas9 protein can have the same parameters for sequence identity as described above for Cas9 proteins. Thus, in some cases, a suitable variant Cas9 protein comprises an amino acid sequence having 4 motifs, each of motifs 1-4 having 60% or more, 70% or more, 75% or more, 80% or more, 85% or more, 90% or more, 95% or more, 99% or more or 100% amino acid sequence identity to motifs 1-4 of the Cas9 amino acid sequence set forth as SEQ ID NO: 5 (the motifs are in Table 1, above, and are set forth as SEQ ID NOs: 1-4, respectively), or to the corresponding portions in any of the amino acid sequences set forth in SEQ ID NOs: 6-816.

In some cases, the variant Cas9 protein is a dCas9 fused to a transcriptional repressor using CRISPRi (e.g., the KRAB domain of Kox1, the CS domain of HP1α, or the WRPW domain of Hes1; see e.g. Gilbert et al., Cell (2013) 154(2):442-451, which is hereby incorporated by reference in its entirety)); or fused to an activation domain (e.g., VP64, p65 and RTA, see e.g., Gilbert et al. ibid) as using CRISPRa.

In some cases, the variant Cas9 protein is a Cas9 nickase and is fused to a base editor. In some cases, the base editor comprises a Cas9 fused to a cytidine deaminase enables a cytidine to uridine conversion resulting in a C to T conversion in a target sequence. In some cases, the base editor comprises a Cas9 fused to an adenine deaminase that enables an adenine to inosine conversion, leading to an A to G conversion in a target sequence (see e.g., Gaudelli et al, Nature (2017) 551:464-471), which is hereby incorporated by reference in its entirety.

### Targeting Moiety

In some cases, a system of the present disclosure comprises an RNA-guided CRISPR/Cas effector polypeptide, where the RNA-guided CRISPR/Cas effector polypeptide is modified to include a targeting moiety that provides for cell type-selective binding or tissue-selective binding. Suitable targeting moieties include but are not limited to, an antibody, a ligand-binding portion of a receptor, or a ligand for a receptor.

The targeting moiety may be used to target the delivery of a cargo RNA to specific cell types, resulting in the delivery of the cargo RNA to the specific targeted cell type. A targeting moiety may selectively bind a target molecule of the target cell, e.g., a target moiety present on the surface of the target cell. For example, the targeting moiety may be an antibody, a receptor, a ligand for a receptor, an aptamer, a small molecule, or a variant thereof.

Any of a variety of cell types can be targeted. Examples include, but are not limited to, stem cells, neurons, epithelial cells and/or dermal cells, adipocytes, cardiomyocytes, renal cells, myocytes, hepatocytes, chondrocytes, islet cells, endothelial cells, dental pulp cells, and osteoblasts. In some cases, the target cell is a diseased cell, e.g., a cancer cell, a virus-infected cell, and the like. In some cases, the target cell is a blood cell, e.g. a T cell, macrophage, NK cell, B cell, megakaryocyte and the like. In some cases, the target is a stem cell including for example a hematopoietic stem cell, a mesenchymal stem cell, a neural stem cell, a pulmonary stem cell, a muscle stem cell and an induced pluripotent stem cell (iPSC).

For example, in some cases, the targeting moiety binds an integrin, where suitable integrins include, e.g., an α1β1, α2β1, α4β1, α5β1, α6β1, αL132, αMβ2, αIIbβ3, αVβ3, αVβ5, αVβ6, or a α6β4 integrin. In some cases, the targeting moiety binds a receptor, such as an EGF receptor (ErbB family), an insulin receptor, a platelet derived growth factor receptor, a fibroblast growth factor receptor, a vascular endothelial growth factor receptor, a human growth factor receptor, a Trk receptor, an Eph receptor, an AXL receptor, an LTK receptor, a TIE receptor, an ROR receptor, a DDR receptor, a RET receptor, a KLG receptor, a RYK receptor, a T cell receptor (TCR), a B cell receptor (BCR), a NK cell receptor (NKR), or a MuSK receptor. In some cases, the targeting moiety binds a G-protein coupled receptor (GPCR), e.g., where the GPCR is a rhodopsin-like receptor, a secretin receptor, a metabotropic glutamate/pheromone receptor, a cyclic AMP receptor, a frizzled/smoothened receptor, CXCR4, CCR5, or a beta-adrenergic receptor.

In some cases, the targeting moiety binds an antigen selected from CEACAM6, c-Met, EGFR, ErbB2, ErbB3, ErbB4, EphA2, IGF1R, GHRHR, GHR, FLT1, KDR, FLT4, CD44v6, CA125, CEA, BTLA, TGFBR2, TGFBR1, IL6R, gp130, TNFR1, TNFR2, PD1, PD-L1, PD-L2, HVEM, mesothelin, PSMA, RANK, ROR1, TNFRSF4, TWEAK-R, HLA, tumor or pathogen derived peptides bound to HLA (such as from hTERT, tyrosinase, or WT-1), LTβR, LIFRβ, LRP5, MUC1, OSMRβ, TCRα, TCRβ, B7H4, TLR7, TLR9, PTCH1, PTCH1, Robol, α-fetoprotein (AFP), and Frizzled.

An antibody can be used as a targeting moiety, e.g., where the antibody is specific for a target molecule on a target cell. For example, in some cases, a targeting moiety is an antibody that binds a tumor-associated or tumor-specific antigen. Some non-limiting examples of tumor antigens include, A33; BAGE; Bcl-2; β-catenin; CA125; CA19-9; CD5; CD19; CD20; CD21; CD22; CD33; CD37; CD45; CD123; CEA; c-Met; CS-1; cyclin B1; DAGE; EBNA; EGFR; ephrinB2; estrogen receptor; FAP; ferritin; folate-binding protein; GAGE; G250; GD-2; GM2; gp75, gp100 (Pmel 17); HER-2/neu; HPV E6; HPV E7; Ki-67; LRP; mesothelin, p53, PRAME; progesterone receptor; PSA; PSMA; MAGE; MART; MUC; MUM-1-B; myc; NYESO-1; ras; RORI; survivin; tenascin; TSTA tyrosinase; VEGF; and WT1. For example, the following cancer cells can be targeted by targeting the associated antigens: leukemia/lymphoma (CD19, CD20, CD22, ROR1, CD33); multiple myeloma (B-cell maturation antigen (BCMA)); prostate cancer (PSMA, WT1, Prostate Stem Cell antigen (PSCA), SV40 T); breast cancer (HER2, ERBB2); stem cell cancer (CD133); ovarian cancer (L1-CAM, extracellular domain of MUC16 (MUC-CD), folate binding protein (folate receptor), Lewis Y); renal cell carcinoma (carboxy-anhydrase-IX (CAIX); melanoma (GD2); and pancreatic cancer (mesothelin, CEA, CD24).

In some cases, the RNA-guided CRISPR/Cas effector polypeptide comprises a targeting moiety that binds hepatocytes, where the targeting moiety binds to asialoglycoprotein receptor (ASGPR). See, e.g., WO 2017/083368; and Rouet et al. (2018) J. Am. Chem. Soc. 140:6596.

In some cases, the targeting moiety binds an antigen present on a virus-infected cell. Examples of viruses include adenoviruses, arenaviruses, bunyaviruses, coronavirusess, flavirviruses, hantaviruses, hepadnaviruses, herpesviruses, papilomaviruses, paramyxoviruses, parvoviruses, picornaviruses, poxviruses, orthomyxoviruses, retroviruses, reoviruses, rhabdoviruses, rotaviruses, spongiform viruses or togaviruses. In other instances, viral antigen markers include peptides expressed by CMV (cytomegalovirus), cold viruses, Epstein-Barr, flu viruses, hepatitis A, B, and C viruses, herpes simplex, HIV, influenza, Japanese encephalitis, measles, polio, rabies, respiratory syncytial, rubella, smallpox, varicella zoster or West Nile virus.

Examples of viral antigens include the following: cytomegaloviral antigens include envelope glycoprotein B and CMV pp65; Epstein-Barr antigens include EBV EBNAI, EBV P18, and EBV P23; hepatitis antigens include the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens include immediate early proteins and glycoprotein D; HIV antigens include gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include hemagglutinin and neuraminidase; Japanese encephalitis viral antigens include proteins E, M-E, M-E-NS1, NS1, and NS1-NS2A; measles antigens include the measles virus fusion protein; rabies antigens include rabies glycoprotein and rabies nucleoprotein; respiratory syncytial viral antigens include the RSV fusion protein and the M2 protein; rotaviral antigens include VP7sc; rubella antigens include proteins E1 and E2; and varicella zoster viral antigens include gpl and gpll.

In some cases, the targeting moiety is an antibody that binds to an antigen present on the surface of a specific cell type. The cell type may be a stem cell, such as a pluripotent stem cell. Some non-limiting examples of antigens specific to pluripotent stem cells include Oct4 and Nanog. In addition to Oct4, Sox2 and Nanog, many other pluripotent stem cell markers have been identified, including Sa114, Dax1, Essrb, Tbx3, Tcl1, Rif1, Nac1 and Zfp281. The targeting moiety may also bind to an antigen of a differentiated cell type. For example, the targeting moiety may bind to an antigen specific for a lung epithelial cell to direct the delivery of a cargo RNA to lung epithelial cells. As a non-limiting example, a targeting moiety may bind to the alveolar epithelial type 1 cell specific protein RTI₄₀ or HTI₅₆ to deliver cargo RNA to alveolar epithelial type 1 cells. As another example, the targeting moiety may bind a mucin, such as muc5ac, or muc5b. It should be appreciated that the examples of antigens provided in this application are not limiting and the targeting moiety may be any moiety capable of binding any cellular antigen known in the art.

### Methods

The present disclosure provides a method of delivering a cargo RNA to a eukaryotic cell. The present disclosure provides a method of delivering a cargo RNA to the cytoplasm of a eukaryotic cell. The methods comprise contacting the eukaryotic cell with a system of the present disclosure. In some cases, the eukaryotic cell is in vitro. In some cases, the eukaryotic cell is in vivo. In some cases, the eukaryotic cell is ex vivo.

The present disclosure provides a method of delivering an RNA-guided effector polypeptide/guide RNA ribonucleoprotein (RNP) to a eukaryotic cell, the method comprising contacting the cell with the system of the present disclosure, where the system comprises: i) a modified guide RNA comprising a guide RNA that is modified to include binding sites for an RBP present in a modified RBP; ii) a modified RBP; and iii) an RNA-guided CRISRP/Cas effector polypeptide, where the RNA-guided CRISRP/Cas effector polypeptide is complexed with the guide RNA. In some cases, the eukaryotic cell is in vitro. In some cases, the eukaryotic cell is in vivo. In some cases, the eukaryotic cell is ex vivo.

In some case, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

In some cases, the eukaryotic cell is in vivo in an organism, and the method comprises administering a system of the present disclosure to the organism. In some cases, the organism is a human. In some cases, the organism is a non-human organism. In some cases, the non-human organism is selected from the group consisting of a plant, a fungus, a non-human mammal, an insect, a reptile, a bird, a fish, a parasite, an arthropod, an invertebrate, and a vertebrate.

Routes of administration that are suitable for use in a method of the present disclosure include various enteral and parenteral routes of administration, including, e.g., intratumoral, peritumoral, intramuscular, intratracheal, intracranial, subcutaneous, intradermal, topical application, intravenous, intraarterial, rectal, nasal, oral, and other enteral and parenteral routes of administration.

A system of the present disclosure can be present in a composition, e.g., a pharmaceutical composition. A composition of the present disclosure can comprise, in addition to a system of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; glycerol; and the like.

The present disclosure provides a composition comprising: a) a system of the present disclosure; and b) a pharmaceutically acceptable excipient. A composition of the present disclosure may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-33 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### ASPECTS A

Aspect 1. A system comprising:

a) a modified RNA-binding protein (RBP) comprising:
i) an RBP; and
ii) one or more endosomolytic peptides covalently linked, directly or via a linker, to the RBP; and

b) a modified cargo RNA complexed to the RBP, wherein the modified cargo RNA comprises a cargo RNA modified to include one or more RBP binding sites that are bound by the RBP present in the modified RBP.

Aspect 2. The system of aspect 1, wherein the RBP comprises an amino acid sequence having at least 85% amino acid sequence identity to the amino acid sequence of an RBP selected from an MS2 polypeptide, a U1A snRNP polypeptide, a PP7 viral coat polypeptide, a poly(A)-binding protein (PABP), a stem-loop binding polypeptide, a boxB polypeptide, a Csy4 polypeptide, a HuA polypeptide, a HuB polypeptide, a HuC polypeptide, a HuD polypeptide, an hnRNP polypeptide, a CArG polypeptide, or an eIF4A polypeptide.

Aspect 3. The system of aspect 1 or aspect 2, wherein the one or more endosomolytic peptides is activated at low pH.

Aspect 4. The system of any one of aspects 1-3, wherein the one or more endosomolytic peptides comprises an amino acid sequence selected from

(SEQ ID NO: 817)
GLFHALLHLLHSLWHLLLHA,
(SEQ ID NO: 818)
GLFEAIAEFIENGWEGLIEGWYGGRKKRRQRRR,
(SEQ ID NO: 819)
GPSQPTYPGDDAPVRDLIRFYRDLRRY,
(SEQ ID NO: 820)
WYSCNVCGKAFVLSRHLNRHLRVHRRAT
and
(SEQ ID NO: 821)
HHEHHEHHEHHEHHEHHEHHEHHEHHE.

Aspect 5. The system of any one of aspects 1-4, wherein the RBP of the modified RBP is linked to the endosomolytic peptide via a cleavable linker.

Aspect 6. The system of aspect 5, wherein the cleavable linker is an enzyme-cleavable linker, an acid-cleavable linker, or a redox-cleavable linker.

Aspect 7. The system of any one of aspects 1-6, wherein the modified RBP comprises 2 endosomolytic peptides.

Aspect 8. The system of any one of aspects 1-6, wherein the modified RBP comprises 3 endosomolytic peptides.

Aspect 9. The system of any one of aspects 1-6, wherein the modified RBP comprises 4 endosomolytic peptides.

Aspect 10. The system of any one of aspects 1-9, wherein the cargo RNA comprises a binding site for a polypeptide other than the modified RBP.

Aspect 11. The system of any one of aspects 1-9, wherein the cargo RNA is a guide RNA comprising:
i) a targeting segment comprising a nucleotide sequence that hybridizes to a nucleotide sequence in a target nucleic acid; and
ii) an activation segment comprising a nucleotide sequence that binds to and activates an RNA-guided effector polypeptide.

Aspect 12. The system of aspect 11, comprising an RNA-guided effector polypeptide.

Aspect 13. The system of aspect 12, wherein the RNA-guided effector polypeptide is a class 2 CRISPR/Cas effector polypeptide.

Aspect 14. The system of aspect 13, wherein the class 2 CRISPR/Cas effector polypeptide is a type II CRISPR/Cas effector polypeptide.

Aspect 15. The system of aspect 13, wherein the class 2 CRISPR/Cas effector polypeptide is a Cas9 protein and the corresponding CRISPR/Cas guide RNA is a Cas9 guide RNA.

Aspect 16. The system of aspect 13, wherein the class 2 CRISPR/Cas effector polypeptide is a type V or type VI CRISPR/Cas effector polypeptide.

Aspect 17. The system of aspect 13, wherein the class 2 CRISPR/Cas effector polypeptide is a Cpf1 protein, a C2c1 protein, a C2c3 protein, or a C2c2 protein.

Aspect 18. The system of aspect 13, wherein the class 2 CRISPR/Cas effector polypeptide is a Cas12 enzyme.

Aspect 19. The system of aspect 13, wherein the class 2 CRISPR/Cas effector polypeptide is a Cas13 enzyme.

Aspect 20. The system of aspect 12, wherein the RNA-guided effector enzyme is a base editor.

Aspect 21. The system of any one of aspects 11-20, wherein the guide RNA comprises one or more nucleic acid modifications.

Aspect 22. The system of aspect 21, wherein the one or more nucleic acid modifications comprise one or more of a modified nucleobase, a modified backbone or non-natural internucleoside linkage, a modified sugar moiety, a Locked Nucleic Acid, and a Peptide Nucleic acid.

Aspect 23. The system of any one of aspects 12-22, wherein the RNA-guided effector enzyme comprises a targeting moiety covalently linked to the RNA-guided effector enzyme.

Aspect 24. The system of aspect 23, wherein the targeting moiety is an antibody, a ligand-binding portion of a receptor, or a ligand for a receptor.

Aspect 25. A method of delivering a cargo RNA to a eukaryotic cell, the method comprising contacting the cell with the system of any one of aspects 1-24.

Aspect 26. A method of delivering an RNA-guided effector polypeptide/guide RNA ribonucleoprotein (RNP) to a eukaryotic cell, the method comprising contacting the cell with the system of any one of aspects 12-24.

Aspect 27. The method of aspect 25 or aspect 26, wherein the eukaryotic cell is in vitro.

Aspect 28. The method of aspect 25 or aspect 26, wherein the eukaryotic cell is in vivo in a human or non-human organism.

Aspect 29. The method of aspect 28, comprising administering the system to the organism.

Aspect 30. The method of any one of aspects 25-29, wherein the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

Aspect 31. The method of aspect 29, wherein the organism is a human.

Aspect 32. The method of aspect 28, wherein the organism is a non-human organism.

Aspect 33. The method of aspect 32, wherein the non-human organism is selected from the group consisting of a plant, a fungus, a non-human mammal, an insect, a reptile, a bird, a fish, a parasite, an arthropod, an invertebrate, and a vertebrate.

Aspects, including embodiments, of the present subject matter described above may be beneficial alone or in combination, with one or more other aspects or embodiments. Without limiting the foregoing description, certain non-limiting aspects of the disclosure numbered 1-39 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### ASPECTS B

Aspect 1. A system comprising:

a) a modified RNA-binding protein (RBP) comprising:
i) an RBP; and
ii) one or more endosomolytic peptides or cell penetrating peptides covalently linked, directly or via a linker, to the RBP; and

b) a modified cargo RNA complexed to the RBP, wherein the modified cargo RNA comprises a cargo RNA modified to include one or more RBP binding sites that are bound by the RBP present in the modified RBP.

Aspect 2. The system of aspect 1, wherein the RBP comprises an amino acid sequence having at least 85% amino acid sequence identity to the amino acid sequence of an RBP selected from an MS2 polypeptide, a U1A snRNP polypeptide, a PP7 viral coat polypeptide, a poly(A)-binding protein (PABP), a stem-loop binding polypeptide, a boxB polypeptide, a Csy4 polypeptide, a HuA polypeptide, a HuB polypeptide, a HuC polypeptide, a HuD polypeptide, an hnRNP polypeptide, a CArG polypeptide, or an eIF4A polypeptide.

Aspect 3. The system of aspect 1 or aspect 2, wherein the one or more endosomolytic peptides or cell penetrating peptides is activated at low pH.

Aspect 4. The system of any one of aspects 1-3, wherein the one or more endosomolytic peptides comprises an amino acid sequence selected from

(SEQ ID NO: 817)
GLFHALLHLLHSLWHLLLHA,
(SEQ ID NO: 818)
GLFEAIAEFIENGWEGLIEGWYGGRKKRRQRRR,
(SEQ ID NO: 819)
GPSQPTYPGDDAPVRDLIRFYRDLRRY,
(SEQ ID NO: 820)
WYSCNVCGKAFVLSRHLNRHLRVHRRAT
and
(SEQ ID NO: 821)
HHEHHEHHEHHEHHEHHEHHEHHEHHE.

Aspect 5. The system of any one of aspects 1-4, wherein the RBP of the modified RBP is linked to the endosomolytic peptide or the cell penetrating peptide via a cleavable linker.

Aspect 6. The system of aspect 5, wherein the cleavable linker is an enzyme-cleavable linker, an acid-cleavable linker, or a redox-cleavable linker.

Aspect 7. The system of any one of aspects 1-6, wherein the modified RBP comprises 1 endosomolytic peptide.

Aspect 8. The system of any one of aspects 1-6, wherein the modified RBP comprises 2 endosomolytic peptides.

Aspect 9. The system of any one of aspects 1-6, wherein the modified RBP comprises 3 endosomolytic peptides.

Aspect 10. The system of any one of aspects 1-6, wherein the modified RBP comprises 4 endosomolytic peptides.

Aspect 11. The system of any one of aspects 1-10, wherein the cargo RNA comprises a binding site for a polypeptide other than the modified RBP.

Aspect 12. The system of any one of aspects 1-10, wherein the cargo RNA is a guide RNA comprising:

i) a targeting segment comprising a nucleotide sequence that hybridizes to a nucleotide sequence in a target nucleic acid; and

ii) an activation segment comprising a nucleotide sequence that binds to and activates an RNA-guided effector polypeptide.

Aspect 13. The system of aspect 12, comprising an RNA-guided effector polypeptide.

Aspect 14. The system of aspect 13, wherein the RNA-guided effector polypeptide is a class 2 CRISPR/Cas effector polypeptide.

Aspect 15. The system of aspect 14, wherein the class 2 CRISPR/Cas effector polypeptide is a type II CRISPR/Cas effector polypeptide.

Aspect 16. The system of aspect 14, wherein the class 2 CRISPR/Cas effector polypeptide is a Cas9 protein and the corresponding CRISPR/Cas guide RNA is a Cas9 guide RNA.

Aspect 17. The system of aspect 14, wherein the class 2 CRISPR/Cas effector polypeptide is a type V or type VI CRISPR/Cas effector polypeptide.

Aspect 18. The system of aspect 14, wherein the class 2 CRISPR/Cas effector polypeptide is a Cpf1 protein, a C2c1 protein, a C2c3 protein, or a C2c2 protein.

Aspect 19. The system of aspect 14, wherein the class 2 CRISPR/Cas effector polypeptide is a Cas12 protein.

Aspect 20. The system of aspect 14, wherein the class 2 CRISPR/Cas effector polypeptide is a Cas13 protein.

Aspect 21. The system of aspect 13, wherein the RNA-guided effector enzyme is a base editor.

Aspect 22. The system of any one of aspects 12-21, wherein the guide RNA comprises one or more nucleic acid modifications.

Aspect 23. The system of aspect 22, wherein the one or more nucleic acid modifications comprise one or more of a modified nucleobase, a modified backbone or non-natural internucleoside linkage, a modified sugar moiety, a Locked Nucleic Acid, and a Peptide Nucleic acid.

Aspect 24. The system of any one of aspects 13-23, wherein the RNA-guided effector enzyme comprises a targeting moiety covalently linked to the RNA-guided effector enzyme.

Aspect 25. The system of aspect 24, wherein the targeting moiety is an antibody, a ligand-binding portion of a receptor, or a ligand for a receptor.

Aspect 26. A method of delivering a cargo RNA to a eukaryotic cell, the method comprising contacting the cell with the system of any one of aspects 1-25.

Aspect 27. A method of delivering an RNA-guided effector polypeptide/guide RNA ribonucleoprotein (RNP) to a eukaryotic cell, the method comprising contacting the cell with the system of any one of aspects 13-25.

Aspect 28. The method of aspect 26 or aspect 27, wherein the eukaryotic cell is in vitro.

Aspect 29. The method of aspect 26 or aspect 27, wherein the eukaryotic cell is in vivo in a human or non-human organism.

Aspect 30. The method of aspect 29, comprising administering the system to the organism.

Aspect 31. The method of any one of aspects 26-30, wherein the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

Aspect 32. The method of aspect 30, wherein the organism is a human.

Aspect 33. The method of aspect 29, wherein the organism is a non-human organism.

Aspect 34. The method of aspect 33, wherein the non-human organism is selected from the group consisting of a plant, a fungus, a non-human mammal, an insect, a reptile, a bird, a fish, a parasite, an arthropod, an invertebrate, and a vertebrate.

Aspect 35. The system of any one of aspects 1-3, wherein the modified RBP comprises 1 cell penetrating peptide.

Aspect 36. The system of any one of aspects 1-3, wherein the modified RBP comprises 2 cell penetrating peptides.

Aspect 37. The system of any one of aspects 1-3, wherein the modified RBP comprises 3 cell penetrating peptides.

Aspect 38. The system of any one of aspects 1-3, wherein the modified RBP comprises 4 cell penetrating peptides.

Aspect 39. The system of any one of aspects 1-3, wherein the one or more cell penetrating peptides comprises an amino acid sequence selected from

(SEQ ID NO: 885)
GRKKRRQRRRPPQ,
(SEQ ID NO: 886)
RQIKIWFQNRRMKWKK,
(SEQ ID NO: 887)
RRIPNRRPRR,
(SEQ ID NO: 888)
RLRWR,
(SEQ ID NO: 889)
GALFLGFLGAAGSTMGAWSQPKKKRKV,
(SEQ ID NO: 890)
KETWWETWWTEWSQPKKRKV,
(SEQ ID NO: 891)
MVRRFLVTLRIRRACGPPRVRV,
(SEQ ID NO: 892)
LLIILRRRIRKQAHAHSK,
(SEQ ID NO: 893)
GWTLNSAGYLLGKINLKALAALAKKIL,
(SEQ ID NO: 894)
QLALQLALQALQAALQLA,
(SEQ ID NO: 895)
DPKGDPKGVTVTVTVTVTGKGDPKPD,
(SEQ ID NO: 896)
RRIRPRPPRLPRPRPRPLPFPRPG,
(SEQ ID NO: 897)
NH₂-HGLASTLTRWAHYNALIRAF-CONH₂,
and
(SEQ ID NO: 898)
WEAALAEALAEALAEHLAEALAEALEALAA.

Aspects, including embodiments, of the present subject matter described above may be beneficial alone or in combination, with one or more other aspects or embodiments. Without limiting the foregoing description, certain non-limiting aspects of the disclosure numbered 1-5 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### ASPECTS C

Aspect 1. A system of the formula: A (X-(L-Z)ₙ)ₘ,

wherein:

i) A is a cargo RNA modified to include one or more binding sites for complexing to one or more modified RNA-binding proteins (RBPs) represented by (X-(L-Z)ₙ)ₘ;

ii) X is an RBP;

iii) L is an optional linking group;

iv) Z is an endosomolytic polypeptide (ELP); and

wherein n and m are each independently an integer from 1 to 10.

Aspect 2. The system of Aspect 1, wherein cargo RNA A comprises 1, 2, 3, 4, 5, 6, 7, 8, 9, or 10 binding sites for binding the modified RBP.

Aspect 3. The system of Aspect 1 or Aspect 2, wherein the RBP comprises an amino acid sequence having at least 85% amino acid sequence identity to the amino acid sequence of an RBP selected from an MS2 polypeptide, a U1A snRNP polypeptide, a PP7 viral coat polypeptide, a poly(A)-binding protein (PABP), a stem-loop binding polypeptide, a boxB polypeptide, a Csy4 polypeptide, a HuA polypeptide, a HuB polypeptide, a HuC polypeptide, a HuD polypeptide, an hnRNP polypeptide, a CArG polypeptide, or an eIF4A polypeptide.

Aspect 4. The system of any one of Aspects 1-3, wherein the ELP comprises an amino acid sequence selected from

(SEQ ID NO: 817)
GLFHALLHLLHSLWHLLLHA,
(SEQ ID NO: 818)
GLFEAIAEFIENGWEGLIEGWYGGRKKRRQRRR,
(SEQ ID NO: 819)
GPSQPTYPGDDAPVRDLIRFYRDLRRY,
(SEQ ID NO: 820)
WYSCNVCGKAFVLSRHLNRHLRVHRRAT
and
(SEQ ID NO: 821)
HHEHHEHHEHHEHHEHHEHHEHHEHHE.

Aspect 5. The system of any one of Aspects 1-4, wherein the cargo RNA is a guide RNA comprising:

i) a targeting segment comprising a nucleotide sequence that hybridizes to a nucleotide sequence in a target nucleic acid; and

ii) an activation segment comprising a nucleotide sequence that binds to and activates an RNA-guided effector polypeptide.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Preparation of Modified ppTG21

The ppTG21 peptide (see e.g. Rittner et al, Mol Ther (2002) 5:104-14) was synthetically modified to include a C-terminal cysteine residue having a pyridyl disulfide leaving group to facilitate conjugation to a cysteine bearing U1A RBP. The modified ppTG21 peptide has the following sequence:

GLFHALLHLLHSLWHLLLHA (C) [pyridyl disulfide] (SEQ ID NO: 817).

### Example 2: Preparation of U1A Construct

The N-terminal RNA-binding domain of human U1A has been modified to include two mutations that improve stability. The Y31 residue was mutated to an H residue and the Q36 residue was mutated to an R residue as described in Oubridge et al, Nature. 1994 Dec. 1; 372(6505):432-8, and as shown below in SEQ ID NO: 848 (mutated residues emphasized):

(SEQ ID NO: 848)
MAVPETRPNHTIYINNLNEKIKKDELKKSLHAIFSRFGQILDILVSRSLK
MRGQAFVIFKEVSSATNALRSMQGFPFYDKPMRIQYAKTDSDIIAKMK

Three mutations were introduced into the U1A plasmid via site-directed mutagenesis to introduce cysteine residues as follows (SEQ ID NO: 823, SEQ ID NO:824, and SEQ ID NO:825):

1 Cys: S71C
2 Cys: S35C, S71C
3 Cys: S35C, S63C, S71C
(SEQ ID NO: 823)
MAVPETRPNHTIYINNLNEKIKKDELKKSLHAIFSRFGQILDILVSRSLK
MRGQAFVIFKEVSSATNALRCMQGFPFYDKPMRIQYAKTDSDIIAKMK
(SEQ ID NO: 824)
MAVPETRPNHTIYINNLNEKIKKDELKKSLHAIFCRFGQILDILVSRSLK
MRGQAFVIFKEVSSATNALRCMQGFPFYDKPMRIQYAKTDSDIIAKMK
(SEQ ID NO: 825)
MAVPETRPNHTIYINNLNEKIKKDELKKSLHAIFCRFGQILDILVSRSLK
MRGQAFVIFKEVCSATNALRCMQGFPFYDKPMRIQYAKTDSDIIAKMK

The three sites of cysteine mutations were on the opposite face of the U1A protein from the RNA-binding site. It has been demonstrated that the U1A protein interacts with RNAs primarily through two RNP motifs (termed RNP1 and RNP2 at the approximate positions 51-59 and 11-17, respectively) and that these two motifs align on one face of the U1A-RNA complex. Positions 35, 63 and 71 appear to face away from the U1A-RNA interface based on the available structural information.

### Example 3: Preparation of the U1A Protein and Conjugation of the ppTG21 ELP Peptides

The modified U1A proteins from Example 2 were used to facilitate attachment of the modified ppTG21 ELP peptides to the U1A protein. The U1A protein and its variants were expressed in purified using a protocol adapted from Ferré D'Amarè et al. (Nature 1998 Oct. 8; 395(6702):567-74). Briefly, following transformation using the plasmid encoding the appropriate U1A plasmid, the U1A protein was recombinantly expressed in E. coli BL21 grown at 37° C. in LB broth supplemented with 100 μg/mL ampicillin. After using starter cultures to inoculate 1 L cultures to O.D.₆₀₀≈0.05, the 1 L cultures were grown to O.D.₆₀₀ 0.6-1.0 and expression was induced by addition of 0.5 mM IPTG. Cells were harvested via centrifugation and resuspended in a buffer consisting of 20 mM Tris, pH 7.4, 500 mM NaCl, 10% Glycerol, and 1 mM TCEP. Following clarification of lysate via centrifugation at 30 k×g for 30′, the U1A variant was purified from the soluble fraction via ammonium sulfate precipitation. Contaminating proteins were removed by centrifugation at 35% ammonium sulfate; U1A variant was isolated by centrifugation at 75% ammonium sulfate. The 75% ammonium sulfate pellet containing U1A variant was resuspended in a buffer consisting of 20 mM HEPES, pH 7.5, 100 mM KCl, 10% Glycerol, 0.5 mM EDTA, and 1 mM TCEP, and dialyzed overnight at 4° C. into an appropriately large volume of the same buffer using 3 kDa MWCO membrane. A 5 mL SP HP column was used to purify the U1A via ion exchange. Ion exchange buffers containing 20 mM HEPES, pH 7.5, 10% Glycerol, 0.5 mM EDTA, and 1 mM TCEP, with the low-salt and high-salt buffers additionally containing 100 mM KCl or 1 M KCl, respectively. The peak resulting from the gradient elution of the ion exchange column was further purified via gel filtration using a 16/60 HiLoad Superdex 75 column with isocratic elution using a buffer consisting of 20 mM HEPES, pH 7.5, 100 mM NaCl (or KCl), 10% Glycerol, 0.5 mM EDTA, and 1 mM TCEP. Pure U1A variant was concentrated (or diluted) to 200 μM, flash-frozen, and stored at −20° C. or −80° C. until use.

The different U1A variants, comprising one, two or three introduced cysteines from Example 2 (SEQ ID NO:s 823, 824 and 825, respectively) were conjugated with modified ppTG21 to obtain U1A-ppTG21 conjugates. To perform a conjugation between a U1A variant and the modified ppTG21 ELP functionalized with a pyridyl-disulfide leaving group, the U1A variant was first desalted using a spin column (e.g. Zeba) to remove the reducing agent in which it was stored. After preparation of the modified ppTG21 powder into a 5 mM stock in DMSO, the U1A variant was mixed with 10 molar equivalents of modified ppTG21 in a buffer ultimately consisting of 20 mM HEPES, pH 7.5, 100 mM NaCl, 10% Glycerol, and 10% DMSO. Conjugation reactions were allowed to proceed ≥12 hours at room temperature. To purify the U1A-ppTG21 conjugates, the sample was centrifuged at 15 k×g for 10′ and the supernatant (containing unconjugated U1A variant and disulfide dimers thereof) was discarded. The resulting gel-like pellet was resuspended in a buffer consisting of 20 mM sodium acetate pH 4.0, 100 mM KCl, and 0.5 mM EDTA. The sample was centrifuged at 15 k×g for 10′, the pellet containing unconjugated modified ppTG21 was discarded, and the supernatant (containing U1A-ppTG21 conjugate) was retained. Conjugation of the ELP to the U1A was then verified by SDS-PAGE (FIG. 8) and intact protein mass spectrometry (FIG. 9). Mass spectrometry results are consistent with intracellular post-translational removal of the N-terminal methionine residue from the U1A variants.

FIG. 8 depicts an SDS-PAGE showing the mobility of U1A and its derivatives that have been conjugated with the pyridyl disulfide-functionalized variant of the ppTG21 peptide. The “no Cys U1A” sample (lane 1) does not have any cysteine residues. Lane 2 shows the 1-Cys U1A sample comprising one cysteine. Lane 3 shows the 2-Cys U1A sample comprising 2 cysteines, and Lane 4 shows the 3-Cys U1A sample comprising 3 cysteines. Shown on the sides of the gel are the locations of the unconjugated, monoconjugated, bisconjugated and trisconjugated U1A derivatives, and demonstrates that the no Cys U1A was not conjugated while the 1-Cys U1A was monoconjugated, the 2-Cys U1A was bisconjugated and the 3-Cys U1A was trisconjugated. Apparent also in the gel are higher molecular weight species from U1A complexes formed from the dimerization of the U1A proteins through disulfide bonds at the introduced cysteine residues.

FIG. 9 depicts a trace from mass spectrometry analysis of the U1A variants shown above, wherein the variants are conjugated with one, two or three chains of ppTG21. The anticipated mass for 1-Cys U1A monoconjugated to a single ppTG21 moiety is 13649.2 Da. The anticipated mass for 2-Cys U1A bisconjugated to two ppTG21 moieties is 16108.2 Da. The anticipated mass for 3-Cys U1A trisconjugated to three ppTG21 moieties is 18567.3 Da. The trace demonstrates that the modified U1A variants display the expected masses. This figure contains overlaid data from three separate and independent mass spectrometry runs.

### Example 4: Production of Guide RNA

Guide RNAs targeting EMX1 (using the same spacer sequence established in Lin et al. (Elife. 2014 Dec. 15; 3:e04766. doi: 10.7554/eLife.04766.)) were produced by in vitro transcription from a template featuring ribozymes flanking the genetic code of the sgRNA (FIG. 10A), as described in Rouet et al. (Rouet et al. J Am Chem Soc 140(21):6596-6603). Transcripts were analyzed by SDS-PAGE stained with SYBR Gold (FIG. 10B). The desired sgRNA transcripts were purified away from other species via preparative denaturing urea PAGE followed by UV shadowing and passive elution. Purity and integrity of the synthesized guide RNA was demonstrated by mass spectrometry (FIG. 11A-FIG. 11B).

Guide RNAs were also prepared to comprise one, two or three introduced stem loops (SL) to serve as U1A binding sites. These sgRNAs were constructed by combining sequence elements described above. The additional stem loops were introduced into the IVT template synthetically prior to the in vitro transcription. The resulting sgRNA sequences are named as follows:

“noSL”:
(SEQ ID NO: 918)
[spacer]GUUUAAGAGCUAUGCUGGAAACAGCAUAGCAAGUUUAAAUAA
GGCUAGUCCGUUAUCAACUUGAAAAAGUGGCACCGAGUCGGUGCUUUUUU
U.
“SLa”:
(SEQ ID NO: 870)
[spacer]GUUUAAGAGCUAUGGAUCAUUGCACUCCGAUCCAUAGCAAGU
UUAAAUAAGGCUAGUCCGUUAUCAACUUGAAAAAGUGGCACCGAGUCGGU
GCUUUUUUU.
“SLb”:
(SEQ ID NO: 871)
[spacer]GUUUAAGAGCUAUGCUGGAAACAGCAUAGCAAGUUUAAAUAA
GGCUAGUCCGUUAUCAACUUGCAAUCCAUUGCACUCCGGAUUGCAAGUGG
CACCGAGUCGGUGCUUUUUUU.
“SLc”:
(SEQ ID NO: 872)
[spacer]GUUUAAGAGCUAUGCUGGAAACAGCAUAGCAAGUUUAAAUAA
GGCUAGUCCGUUAUCAACUUGAAAAAGUGGCACCGAGUCGGUGCAUCAUC
GCGCUGCAUUGCACUCCGCAGCGCUUUUUUU.
“SLab”:
(SEQ ID NO: 919)
[spacer]GUUUAAGAGCUAUGGAUCAUUGCACUCCGAUCCAUAGCAAGU
UUAAAUAAGGCUAGUCCGUUAUCAACUUGCAAUCCAUUGCACUCCGGAUU
GCAAGUGGCACCGAGUCGGUGCUUUUUUU.
“SLac”:
(SEQ ID NO: 920)
GUUUAAGAGCUAUGGAUCAUUGCACUCCGAUCCAUAGCAAGUUUAAAUAA
GGCUAGUCCGUUAUCAACUUGAAAAAGUGGCACCGAGUCGGUGCAUCAUC
GCGCUGCAUUGCACUCCGCAGCGCUUUUUUU.
“SLbc”:
(SEQ ID NO: 921)
[spacer]GUUUAAGAGCUAUGCUGGAAACAGCAUAGCAAGUUUAAAUAA
GGCUAGUCCGUUAUCAACUUGCAAUCCAUUGCACUCCGGAUUGCAAGUGG
CACCGAGUCGGUGCAUCAUCGCGCUGCAUUGCACUCCGCAGCGCUUUUUU
U.
“SLabc”:
(SEQ ID NO: 922)
[spacer]GUUUAAGAGCUAUGGAUCAUUGCACUCCGAUCCAUAGCAAGU
UUAAAUAAGGCUAGUCCGUUAUCAACUUGCAAUCCAUUGCACUCCGGAUU
GCAAGUGGCACCGAGUCGGUGCAUCAUCGCGCUGCAUUGCACUCCGCAGC
GCUUUUUUU.
“SLaa”:
(SEQ ID NO: 873)
[spacer]GUUUAAGAGCUAUGCGAGGCUAAGUCGCAUUGCACUCCGCGA
CAGCACAAGCCCGCUGCCAUUGCACUCCGGCAGCAGGGAACUCGCAUAGC
AAGUUUAAAUAAGGCUAGUCCGUUAUCAACUUGAAAAAGUGGCACCGAGU
CGGUGCUUUUUUU.
“SLbb”:
(SEQ ID NO: 874)
[spacer]GUUUAAGAGCUAUGCUGGAAACAGCAUAGCAAGUUUAAAUAA
GGCUAGUCCGUUAUCAACUUGCGAGGCUAAGUCGCAUUGCACUCCGCGAC
AGCACAAGCCCGCUGCCAUUGCACUCCGGCAGCAGGGAACUCGCAAGUGG
CACCGAGUCGGUGCUUUUUUU.
“SLbub”:
(SEQ ID NO: 923)
[spacer]GUUUAAGAGCUAUGCUGGAAACAGCAUAGCAAGUUUAAAUAA
GGCUAGUCCGUUAUCAACUUGUCGAGGCUAAGUCGCAUUGCACUCCGCGA
CAGCACAAGCCCGCUGCCAUUGCACUCCGGCAGCAGGGAACUCGUCAAGU
GGCACCGAGUCGGUGCUUUUUUU.
“SLaab”:
(SEQ ID NO: 924)
[spacer]GUUUAAGAGCUAUGCGAGGCUAAGUCGCAUUGCACUCCGCGA
CAGCACAAGCCCGCUGCCAUUGCACUCCGGCAGCAGGGAACUCGCAUAGC
AAGUUUAAAUAAGGCUAGUCCGUUAUCAACUUGCAAUCCAUUGCACUCCG
GAUUGCAAGUGGCACCGAGUCGGUGCUUUUUUU.
“SLabb”:
(SEQ ID NO: 925)
[spacer]GUUUAAGAGCUAUGGAUCAUUGCACUCCGAUCCAUAGCAAGU
UUAAAUAAGGCUAGUCCGUUAUCAACUUGCGAGGCUAAGUCGCAUUGCAC
UCCGCGACAGCACAAGCCCGCUGCCAUUGCACUCCGGCAGCAGGGAACUC
GCAAGUGGCACCGAGUCGGUGCUUUUUUU.
“SLabub”:
(SEQ ID NO: 926)
[spacer]GUUUAAGAGCUAUGGAUCAUUGCACUCCGAUCCAUAGCAAGU
UUAAAUAAGGCUAGUCCGUUAUCAACUUGUCGAGGCUAAGUCGCAUUGCA
CUCCGCGACAGCACAAGCCCGCUGCCAUUGCACUCCGGCAGCAGGGAACU
CGUCAAGUGGCACCGAGUCGGUGCUUUUUUU.

Note that the designation “noSL RNP” refers to the use of the noSL sgRNA formed into an RNP complex with Cas9 protein. The designation “[spacer]” refers to the variable, targeting region of the sgRNA variant to be synthesized. In the Examples presented herein, this region consists of 20 nt, although this need not be the case.

FIGS. 10A-10B depict the strategy and results for guide RNA production. FIG. 10A is a schematic representing the dual-ribozyme approach employed with in vitro transcription (IVT) to synthesize high-quality, high-purity single guide RNAs of lengths that are difficult to obtain via solid-state synthesis (Rouet et al. J Am Chem Soc 140(21):6596-6603). FIG. 10B depicts an analytical denaturing 4-20% polyacrylamide urea-PAGE gel revealing the products of various guide RNA IVT reactions bearing stem loop modifications at different positions of the sgRNA scaffold. Arrows on the right side of the gel indicate the identity of each major species present in the sample. The sgRNA variant samples vary in size from 111 nt to 143 nt. As described in (Rouet, ibid), a preparative denaturing PAGE gel allowed the guide RNA bands to be cut out and eluted, and provided pure guide RNA that was free of contaminating ribozymes or any other RNA species.

FIGS. 11A-11B depict mass spectrometry traces of the guide RNAs. FIG. 11A shows a Denaturing Urea PAGE gel (inset) and mass spectrometry result demonstrating purity and integrity of IVT-synthesized unmodified guide RNA noSL (expected molecular weight of 36,390 Da) targeting the EMX1 gene (spacer sequence GUCACCUCCAAUGACUAGGG (SEQ ID NO:917)). Artifactual peaks with larger apparent molecular weights than 36,390 Da resulted from salt adducts, and were not indicative of covalent modification of the guide RNA. FIG. 11B is a mass spectrometry trace demonstrating the purify purity and integrity of IVT-synthesized guide RNA (SLabub) that was engineered to bind U1A at three sites and is targeting the EMX1 gene (spacer sequence GUCACCUCCAAUGACUAGGG (SEQ ID NO:917)).

### Example 5: Binding of Conjugated ELP-U1A Variants to Guide RNAs or RNP

Cas9 was purified as described in Rouet et al. (ibid). The purified Cas9 (with or without conjugation by ASGPr ligand) was mixed with a variant guide RNA (i.e., a guide RNA with or without one or more SL, as described above), as described in Rouet et al. (ibid). Briefly, Cas9 protein was diluted to the desired concentration (25 μM, 12 μM, or 10 μM) with a final magnesium chloride concentration of 1 mM. The sgRNA was prepared in a buffer without magnesium chloride and refolded by heating to 95° C. followed by rapid cooling to 5° C. The prepared Cas9 solution was added to the prepared solution of sgRNA at the desired concentration (30 μM, 14.4 μM, or 12 μM) in a 1:1.2 Cas9:RNA molar ratio and the resulting solution was incubated at 37° C. for 10 min. The resulting RNP was cooled to room temperature and was either used immediately or frozen at −80° C. for later use. Guides or RNP were used to test their capacity to be bound by U1A variants and U1A-ppTG21 conjugates by fluorescence polarization binding assays using the experimental design described in Hochstrasser et al. Mol Cell. 2016 Sep. 1; 63(5):840-51. doi: 10.1016/j.molcel.2016.07.027. In these experiments, the guides comprising a single stem loop (SLa and SLb), or three stem loops (SLabc) were compared with guides lacking any introduced stem loop (noSL). The results (FIGS. 12A-12B) demonstrate that the addition of the stem loops resulted in an increase in affinity for the U1A adapter protein, in two contexts: sgRNA alone or sgRNA complexed with Cas9 to form an RNP. Another set of experiments compared the same properties of the SLb and SLabub sgRNA configurations, either as sgRNA alone or as sgRNA complexed with Cas9 to form an RNP. The results (FIGS. 13A-13B) demonstrated that the binding of the U1A variants was increased in the presence of the stem loops.

Biolayer interferometry (BLI) was used to assess the capacity of Cas9 RNPs containing sgRNA variants to bind U1A (or U1A-ppTG21 conjugates) as well as to assess the persistence of any binding events. These assays approximated the experimental design described in Richardson et al. (Nat Biotechnol. 2016 March; 34(3):339-44. doi: 10.1038/nbt.3481). These experiments relied on a Cas9 protein covalently, site-specifically labeled with a biotin moiety, allowing loading onto a BLI sensor bearing streptavidin. The instrument used was an Octet Red 384. The buffer used was 20 mM HEPES, pH 7.5, 100 mM KCl, 10% Glycerol, 0.01 mg/ml Heparin, 0.01% IGEPAL. The loading step of the experiment used Cas9 RNP at 20 nM in the wells. U1A wells contained 100 nM, 330 nM, or 1 μM U1A. An experiment with noSL RNP immobilized on the sensor demonstrated a lack of substantial U1A binding to the Cas9 RNP (FIG. 14). In contrast to the noSL RNP experiment, the SLabub RNP was markedly bound by the U1A, and the binding persisted for the ˜2000 s (˜33 min) duration of the dissociation phase (FIG. 15A). The SLabub RNP experiment was repeated using U1A-ppTG21 conjugate in the wells at concentrations spanning 100 nM to 6 μM (FIG. 15B). Pronounced binding was observed at all concentrations, and persisting binding was observed over the ˜900 s (˜15 min) duration of the dissociation phase.

To ensure that the binding observed was specific to the interfacial RNA nucleotides (GCACUC) (SEQ ID NO:929) known to be recognized by U1A, additional BLI experiments were performed comparing two different RNA sequences: the canonical U1 RNA stem loop 2 (AAUCCAUUGCACUCCGGAUU, SEQ ID NO:927) and the negative-control U2 stem loop (AAUCCAUUCAUGUACCGGAUU, SEQ ID NO:928) known to exhibit no specific binding by U1A (see e.g. Scherly et al, Nature (1990) 345(6275:502-506). Both RNA sequences were synthesized with a 5′ biotin modification to facilitate loading onto a BLI sensor bearing streptavidin. The loading step of this experiment contained 40 nM of the desired RNA stem loop in the wells. The concentrations of U1A-ppTG21 conjugate in the wells covered the range of 0.33-333 nM. Substantial binding was observed between the U1 stem loop and the U1A-ppTG21 conjugate, and this binding persisted for the ˜2000 s (˜33 min) of the dissociation phase (FIG. 15C). In contrast, the U2 stem loop was not substantially bound by the U1A-ppTG21 conjugate (FIG. 15D).

FIGS. 12A-12B are graphs from fluorescence polarization (FP) binding assays. These tested for affinity between adaptor protein U1A and different guide RNA constructs. FP assays were performed using 1-Cys U1A monoconjugated with maleimide-functionalized fluorescein present at 10 nM and specified guide RNAs or RNPs were present at the shown concentration values. The sgRNAs were prepared via dual-ribozyme in vitro transcription as described above in FIGS. 10A-10B. FIG. 12A shows an FP assay detecting binding between U1A and guide RNA constructs: noSL is the unmodified guide with no binding sites for U1A; SLa, SLb, and SLc each contain a single binding site for U1A; SLabc contains three binding sites for U1A. Curve fitting provides approximate binding affinities: 8.3 nM for U1A:SLa binding; 8.8 nM for U1A:SLb binding; 10.4 nM for U1A:SLc binding; 0.9 nM for U1A:SLabc binding. FIG. 12B shows an FP assay detecting binding between U1A and Cas9-sgRNA (RNP) complexes. The same four guide RNAs described in (a) were assembled into RNP complexes as described in (Rouet, ibid). Curve fitting provided approximate binding affinities: 15.8 nM for U1A:SLa-RNP binding; 14.3 nM for U1A:SLb-RNP binding; 13.8 nM for U1A:SLc-RNP binding; 3.6 nM for U1A:SLabc-RNP binding.

FIGS. 13A-13B are graphs from FP binding assays testing for affinity between adaptor protein U1A and different guide RNA constructs. FP assays were performed as in FIG. 12, but with different guide RNAs. 1-Cys U1A monoconjugated with maleimide-functionalized fluorescein was present at 10 nM and specified guide RNAs or RNPs were present at the shown concentration values. FIG. 13A is an FP assay detecting binding between U1A and guide RNA constructs: noSL is the unmodified guide with no binding sites for U1A; SLb contains a single binding site for U1A; SLabub contains three binding sites for U1A. Curve fitting provides approximate binding affinities: 2.2 nM for U1A:SLb binding; 1.2 nM for U1A:SLabub binding. FIG. 13B is an FP assay detecting binding between U1A and Cas9-sgRNA (RNP) complexes. Curve fitting provides approximate binding affinities: 0.08 nM for U1A:SLb-RNP binding; 0.15 nM for U1A:SLabub-RNP binding.

FIGS. 15A-15D is a trace from a biolayer interferometry (BLI) results demonstrating persisting binding between U1A and a Cas9 RNP containing an engineered guide RNA bearing three binding sites for the U1A adaptor (noSL). The experiment relied on a Cas9 protein covalently labeled with a biotin, allowing loading onto a BLI sensor bearing streptavidin. The concentrations of U1A samples in the wells is indicated next to each curve in a corresponding shade of grey.

### Example 6: Genome Editing Using U1A Variants and Modified Guide RNAs

Genome editing was performed as described in Rouet et al. (ibid), in particular the genome editing relying on the 1NLS Cas9 construct with or without the ASGPr ligand, with the following changes throughout: non-coated plates were used instead of gelatin-coated plates; 50 pmol of RNP was added per 100,000 cells in each condition (e.g. 10 pmol per 20,000 cells in a 96-well plate format); after heteroduplex formation with 200 ng of genomic DNA extracted from the cells, the T7E1 assay was used for editing analysis with polyacrylamide gels (Novex TBE 4-20%) using SYBR Gold staining. Gels were imaged with a Gel Doc transilluminator (BioRad) and quantification was based on relative band intensities. Indel percentage was determined by the formula: 100×(1−(1−(b+c)/(a+b+c))¹¹², where a is the intensity of undigested PCR product, and b and c are the intensities of the cleaved products (see Guschin et al. (2010) in Engineered Zinc Finger Proteins, eds Mackay J P, Segal D J (Humana Press, Totowa, N.J.), pp 247-256). Cas9 RNP was prepared as in Example 5 and applied either alone, with addition of ppTG21, or with addition U1A-ppTG21 conjugates. Cas9 RNP mixtures were added to cells and returned to the incubator. Cells were harvested 44-48 h later, and genomic DNA was harvested. T7E1 analysis was performed as described in Rouet et al. (ibid).

An editing experiment utilized Cas9 conjugated with two copies of the ASGPr ligand, which is known to induce endocytosis of the conjugated Cas9 (when it has bound a sgRNA to form an RNP complex) into HepG2 cells, which bear the ASGP receptor (Rouet et al. (2018) J. Am. Chem. Soc. 140:6596). This experiment used ligand-bound Cas9 RNP and assessed the genome editing ability of different configurations of variant sgRNA (e.g. noSL, SLa, SLb, SLab, or SLabub, which bear 0, 1, 1, 2, or 3 binding sites for U1A, respectively) bound by different U1A-ppTG21 conjugates (e.g. U1A(1), U1A(2), or U1A(3), which respectively represent U1A monoconjugated, bisconjugated, or trisconjugated with ppTG21, as visualized in FIG. 4). For each condition containing U1A-ppTG21, 1.5 molar equivalents of U1A-ppTG21 were added per mole of U1A binding site on the corresponding variant sgRNA. SLa and SLb conditions contained 1.5 molar equivalents of U1A-ppTG21; SLab conditions contained 3 molar equivalents of U1A-ppTG21; SLabub conditions contained 4.5 molar equivalents of U1A-ppTG21; noSL RNP was supplemented with 1.5 molar equivalents of U1A-ppTG21. Genome editing targeted the EMX1 locus. The resulting genome editing rates revealed pronounced genome editing by the SLabub RNP constructs irrespective of the U1A-ppTG21 conjugate used, and increased editing in other RNP constructs (especially SLab) as the extent of ppTG21 conjugation increased (FIG. 4).

Another editing experiment was performed to assess the ability of the Cas9 RNP with adaptor-recruited ELPs (arELP) to perform ligand-enhanced (and thus, receptor-mediated) genome editing. This experiment compared Cas9 RNP without the ASGPr ligand to Cas9 RNP bisconjugated to the ASGPr ligand (FIGS. 16A-16E). Genome editing targeted the EMX1 locus. Positive (FIG. 16A) and negative (FIG. 16B) controls respectively established the maximal and background editing rates for these conditions. An experiment involving 30 molar equivalents of ppTG21 added to Cas9 RNP in trans approximated the ligand-mediated editing experiment of Rouet et al. (ibid) but with the present conditions and changes as noted above (FIG. 16C). An experiment combining SLabub RNP (either with or without ASGPr ligand) with a monoconjugated U1A-ppTG21 (the U1A variant bearing a single Cys, conjugated to a single moiety of ppTG21) reveals that this strategy is capable of promoting ligand-enhanced (and thus, receptor-mediated) genome editing (FIG. 16D). An additional control tests the editing activity of a noSL RNP (incapable of recruiting U1A-ppTG21) bisconjugated to the ASGPr ligand; no substantial genome editing is observed when the RNP is not capable of recruiting the U1A-ppTG21(FIG. 16E). SLabub conditions contained 4.5 molar equivalents of U1A-ppTG21; noSL RNP was supplemented with 1.5 molar equivalents of U1A-ppTG21.

FIG. 4 illustrates EMX1 editing results in HEPG2 cells, which were treated with a positive control for each RNP construct (“noSL” conditions), and RNPs containing various sgRNA designs capable of recruiting various numbers of U1A RBP adaptor molecules to their engineered U1A binding sites, wherein the U1A RBP adaptor molecules include 1, 2, or 3 covalently bound ELPs. The recruitment of more ELPs to the RNP surface, via U1A association, promotes drastically improved editing as compared to the “noSL” conditions, which represent an RNP that cannot recruit U1A (or its bound ELPs). In each grouping, the bars are as follows: left most bar: noSL; second to left bar: SLa; middle bar: SLb; second to right bar: SLab; right most bar: SLabub. Note that the predicted secondary stem loop structures of each of the RNAs is shown below the graph, indicating their identities. The legend shows how many U1A conjugates are expected to be bound to each RNP based on the number of U1A binding sites incorporated into each sgRNA variant: 0 for noSL; 1 for SLa or SLb; 2 for SLab; 3 for SLabub. To promote complete binding, each binding site was provided with 1.5 molar equivalents of U1A conjugate: 1.5 equivalents per SLa or SLb RNP; 3 equivalents per SLab RNP; 4.5 equivalents per SLabub RNP. The noSL RNP condition included 4.5 molar equivalents—the highest number of molar equivalents; the intention was to test for editing activity that does not depend on specific association between U1A conjugate and the U1A binding sites incorporated into the “SLx” sgRNA variants. The leftmost “CPP” lanes represent a positive control for genome editing facilitated by 30 molar equivalents of a commercially available cell-penetrating peptide.

FIG. 14 is a trace from a biolayer interferometry (BLI) experiment, and demonstrated the absence of substantial binding between U1A and a Cas9 RNP containing an unmodified guide RNA (noSL). The experiment relied on a Cas9 protein covalently labeled with a biotin, allowing loading onto a BLI sensor bearing streptavidin. The concentrations of U1A samples in the wells is indicated next to each curve in a corresponding shade of grey.

FIGS. 16A-16E is a graph demonstrating receptor-mediated genome editing of the EMX1 gene via adaptor-recruited endosomolytic peptides (arELP) bound to Cas9 RNP. Gels were imaged with a Gel Doc transilluminator (BioRad) and quantification was based on relative band intensities. Detection of insertions and deletions in the edited target (“indels”) caused by error prone non-homologous end-joining follow cleavage was used as a measure of editing efficiency. Indel percentage was determined by the formula: 100×(1-(1-(b+c)/(a+b+c))½, where a is the intensity of undigested PCR product, and b and c are the intensities of the cleaved products. Experiments were performed either without (unfilled/white bars) or with (filled bars) bisconjugation of the asialogycoprotein ligand (ASGPrL, ligand) to the Cas9 protein component as described in (Rouet, ibid). Shown below the graph are schematics depicting the RNP complexes. Group A shows Cas9 RNP containing unmodified sgRNA, with or without ligand and electroporated into cells. Group B shows the results from Cas9 RNP containing unmodified sgRNA, with or without ligand, incubated with cells in the absence of any endosomolytic reagent. Group C shows the results from Cas9 RNP containing unmodified sgRNA, with or without ligand, in the presence of 30 molar equivalents (1500 pmol) of the endosomolytic peptide (ELP) ppTG21. Group D shows the results from Cas9 RNP containing sgRNA that had been modified to include three stem-loops (SLabub) to recruit adaptor-recruited endosomolytic peptides (arELP), using Cas9 protein with or without ligand, in the presence of 4.5 molar equivalents (270 pmol) of 1-Cys U1A adaptor protein monoconjugated to a pyridyl disulfide-functionalized variant of the ppTG21 peptide. Group E shows the results from Cas9 RNP containing unmodified sgRNA, with ligand, in the presence of 4.5 molar equivalents (270 pmol; 1.5 equivalents per U1A binding site in the sgRNA) of 1-Cys U1A adaptor protein monoconjugated to a pyridyl disulfide-functionalized variant of the ppTG21 peptide. In all groupings, white inlaid text on the data bar refers to the approximate fold enhancement provided by the presence of the ligand; an indicator of the receptor-mediated fidelity of the delivery strategy.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A system comprising:
a) a modified RNA-binding protein (RBP) comprising:
i) an RBP; and
ii) one or more endosomolytic peptides or cell penetrating peptides covalently linked, directly or via a linker, to the RBP; and
b) a modified cargo RNA complexed to the RBP, wherein the modified cargo RNA comprises a cargo RNA modified to include one or more RBP binding sites that are bound by the RBP present in the modified RBP.

2. The system of claim 1, wherein the RBP comprises an amino acid sequence having at least 85% amino acid sequence identity to the amino acid sequence of an RBP selected from an MS2 polypeptide, a U1A snRNP polypeptide, a PP7 viral coat polypeptide, a poly(A)-binding protein (PABP), a stem-loop binding polypeptide, a boxB polypeptide, a Csy4 polypeptide, a HuA polypeptide, a HuB polypeptide, a HuC polypeptide, a HuD polypeptide, an hnRNP polypeptide, a CArG polypeptide, or an eIF4A polypeptide.

3. The system of claim 1 or claim 2, wherein the one or more endosomolytic peptides or cell penetrating peptides is activated at low pH.

4. The system of any one of claims 1-3, wherein the one or more endosomolytic peptides comprises an amino acid sequence selected from GLFHALLHLLHSLWHLLLHA (SEQ ID NO: 817), GLFEAIAEFIENGWEGLIEGWYGGRKKRRQRRR (SEQ ID NO: 818), GPSQPTYPGDDAPVRDLIRFYRDLRRY (SEQ ID NO: 819), WYSCNVCGKAFVLSRHLNRHLRVHRRAT (SEQ ID NO: 820) and HHEHHEHHEHHEHHEHHEHHEHHEHHE (SEQ ID NO: 821).

5. The system of any one of claims 1-4, wherein the RBP of the modified RBP is linked to the endosomolytic peptide or the cell penetrating peptide via a cleavable linker.

6. The system of claim 5, wherein the cleavable linker is an enzyme-cleavable linker, an acid-cleavable linker, or a redox-cleavable linker.

7. The system of any one of claims 1-6, wherein the modified RBP comprises 1 endosomolytic peptide.

8. The system of any one of claims 1-6, wherein the modified RBP comprises 2 endosomolytic peptides.

9. The system of any one of claims 1-6, wherein the modified RBP comprises 3 endosomolytic peptides.

10. The system of any one of claims 1-6, wherein the modified RBP comprises 4 endosomolytic peptides.

11. The system of any one of claims 1-10, wherein the cargo RNA comprises a binding site for a polypeptide other than the modified RBP.

12. The system of any one of claims 1-10, wherein the cargo RNA is a guide RNA comprising:
i) a targeting segment comprising a nucleotide sequence that hybridizes to a nucleotide sequence in a target nucleic acid; and
ii) an activation segment comprising a nucleotide sequence that binds to and activates an RNA-guided effector polypeptide.

13. The system of claim 12, comprising an RNA-guided effector polypeptide.

14. The system of claim 13, wherein the RNA-guided effector polypeptide is a class 2 CRISPR/Cas effector polypeptide.

15. The system of claim 14, wherein the class 2 CRISPR/Cas effector polypeptide is a type II CRISPR/Cas effector polypeptide.

16. The system of claim 14, wherein the class 2 CRISPR/Cas effector polypeptide is a Cas9 protein and the corresponding CRISPR/Cas guide RNA is a Cas9 guide RNA.

17. The system of claim 14, wherein the class 2 CRISPR/Cas effector polypeptide is a type V or type VI CRISPR/Cas effector polypeptide.

18. The system of claim 14, wherein the class 2 CRISPR/Cas effector polypeptide is a Cpf1 protein, a C2c1 protein, a C2c3 protein, or a C2c2 protein.

19. The system of claim 14, wherein the class 2 CRISPR/Cas effector polypeptide is a Cas12 protein.

20. The system of claim 14, wherein the class 2 CRISPR/Cas effector polypeptide is a Cas13 protein.

21. The system of claim 13, wherein the RNA-guided effector enzyme is a base editor.

22. The system of any one of claims 12-21, wherein the guide RNA comprises one or more nucleic acid modifications.

23. The system of claim 22, wherein the one or more nucleic acid modifications comprise one or more of a modified nucleobase, a modified backbone or non-natural internucleoside linkage, a modified sugar moiety, a Locked Nucleic Acid, and a Peptide Nucleic acid.

24. The system of any one of claims 13-23, wherein the RNA-guided effector enzyme comprises a targeting moiety covalently linked to the RNA-guided effector enzyme.

25. The system of claim 24, wherein the targeting moiety is an antibody, a ligand-binding portion of a receptor, or a ligand for a receptor.

26. A method of delivering a cargo RNA to a eukaryotic cell, the method comprising contacting the cell with the system of any one of claims 1-25.

27. A method of delivering an RNA-guided effector polypeptide/guide RNA ribonucleoprotein (RNP) to a eukaryotic cell, the method comprising contacting the cell with the system of any one of claims 13-25.

28. The method of claim 26 or claim 27, wherein the eukaryotic cell is in vitro.

29. The method of claim 26 or claim 27, wherein the eukaryotic cell is in vivo in a human or non-human organism.

30. The method of claim 29, comprising administering the system to the organism.

31. The method of any one of claims 26-30, wherein the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

32. The method of claim 30, wherein the organism is a human.

33. The method of claim 29, wherein the organism is a non-human organism.

34. The method of claim 33, wherein the non-human organism is selected from the group consisting of a plant, a fungus, a non-human mammal, an insect, a reptile, a bird, a fish, a parasite, an arthropod, an invertebrate, and a vertebrate.

35. The system of any one of claims 1-3, wherein the modified RBP comprises 1 cell penetrating peptide.

36. The system of any one of claims 1-3, wherein the modified RBP comprises 2 cell penetrating peptides.

37. The system of any one of claims 1-3, wherein the modified RBP comprises 3 cell penetrating peptides.

38. The system of any one of claims 1-3, wherein the modified RBP comprises 4 cell penetrating peptides.

39. The system of any one of claims 1-3, wherein the one or more cell penetrating peptides comprises an amino acid sequence selected from
(SEQ ID NO: 885)
GRKKRRQRRRPPQ,
(SEQ ID NO: 886)
RQIKIWFQNRRMKWKK,
(SEQ ID NO: 887)
RRIPNRRPRR,
(SEQ ID NO: 888)
RLRWR,
(SEQ ID NO: 889)
GALFLGFLGAAGSTMGAWSQPKKKRKV,
(SEQ ID NO: 890)
KETWWETWWTEWSQPKKRKV,
(SEQ ID NO: 891)
MVRRFLVTLRIRRACGPPRVRV,
(SEQ ID NO: 892)
LLIILRRRIRKQAHAHSK,
(SEQ ID NO: 893)
GWTLNSAGYLLGKINLKALAALAKKIL,
(SEQ ID NO: 894)
QLALQLALQALQAALQLA,
(SEQ ID NO: 895)
DPKGDPKGVTVTVTVTVTGKGDPKPD,
(SEQ ID NO: 896)
RRIRPRPPRLPRPRPRPLPFPRPG,
(SEQ ID NO: 897)
NH₂-HGLASTLTRWAHYNALIRAF-CONH₂,
and
(SEQ ID NO: 898)
WEAALAEALAEALAEHLAEALAEALEALAA.
